(19) United States
(12) Patent Application Publication
(10) Pub. No.: US 2014/0038842 A1
(43) Pub. Date: Feb. 6, 2014

(54) CELL SURFACE DISPLAY USING PDZ DOMAINS

(71) Applicants: Eric M. Tam, Coquitlam, (CA); Isaac J. Rondon, San Francisco, CA (US); Chao B. Huang, San Leandro, CA (US); Violet Votin, Pleasant Hill, CA (US)

(72) Inventors: Eric M. Tam, Coquitlam, (CA); Isaac J. Rondon, San Francisco, CA (US); Chao B. Huang, San Leandro, CA (US); Violet Votin, Pleasant Hill, CA (US)

(73) Assignee: XOMA TECHNOLOGY, Berkeley, CA (US)

(21) Appl. No.: 13/995,611

(22) PCT Filed: Dec. 28, 2011
(86) PCT No.: PCT/US11/67482

§ 371 Date: Oct. 9, 2013

Related U.S. Application Data
(60) Provisional application No. 61/427,722, filed on Dec. 28, 2010.
(60) Provisional application No. 61/532,463, filed on Sep. 8, 2011.
(60) Provisional application No. 61/566,440, filed on Dec. 2, 2011.

(51) Int. Cl. C12N 15/10; C07K 16/00

(52) U.S. Cl. CPC C12N 15/1037; C07K 16/00
USPC 506/9; 435/254.2; 435/254.21; 435/254.23; 435/254.22; 435/252.3; 435/252.33; 435/252.31; 435/252.34; 435/328; 506/14; 506/26

(57) ABSTRACT

Novel materials and methods useful for displaying polypeptides on the surface of a cell are provided, including cell surface proteins fused to a PDZ domain peptide and antibodies fused to PDZ-binding peptides.

### CROSS REFERENCE TO RELATED APPLICATIONS

This application claims priority to U.S. Provisional Application No. 61/427,722, filed Dec. 28, 2010, U.S. Provisional Application No. 61/532,463, filed Sep. 8, 2011, and U.S. Provisional Application No. 61/566,440, filed Dec. 2, 2011.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: One 935,414 byte ASCII (Text) file named “45636A_SeqListing.txt” created on Dec. 28, 2011.

### FIELD OF THE INVENTION

The invention relates to materials and methods useful for displaying proteins, including antibodies, on the surface of a cell.

### BACKGROUND

Display of peptides on the surface of filamentous bacteriophage, or phage display, has proven a versatile and effective methodology for the isolation of peptide ligands binding to a diverse range of targets. Phage display involves the localization of polypeptides as terminal fusions to the coat proteins, e.g., pIII, pVIII of bacteriophage particles. See Scott, J. K. and G. P. Smith (1990) Science 249(4967):386-390; and Lowman, H. B., et al. (1991) Biochem. 30(45):10832-10838. Generally, polypeptides that bind to the target of interest are isolated by incubating with a target, washing away non-binding phage, eluting the bound phage, and then re-amplifying the phage population by infecting a fresh culture of bacteria. Phage display is limited to about a few thousand copies of the displayed polypeptide per phage or less, far less (one to five copies) when pIII is the coat protein utilized for display, thereby precluding the use of sensitive fluorescence-activated cell sorting (FACS) methodologies for isolating the desired sequences. Moreover, phage can be difficult to elute or recover from an immobilized target ligand, thereby resulting in clonal loss.

It has been reported that polypeptides can be linked to yeast cell wall proteins and displayed on yeast cells (reviewed in Feldhaus and Siegel, J. Immunol. Methods, 290, 69-80 (2004); Wang et al., J. Immunol. Methods, 354, 11-19 (2010)).

PDZ domains are modular protein interaction domains that play a role in protein targeting and protein complex assembly. The structural features of PDZ domains allow them to mediate specific protein-protein interactions that underlie the assembly of large protein complexes involved in signaling or subcellular transport. Structurally, PDZ domains are composed of a 5- to 6-stranded anti-parallel β-barrel and 2-3 α-helices. PDZ domains typically recognize short sequences located at the C-termini of target proteins, although some PDZ domains are known to recognize internal sequences.

### SUMMARY OF THE INVENTION

This disclosure relates to methods and materials useful for displaying proteins-of-interest, including antibodies. Eukaryotic (including yeast and mammalian cells) and prokaryotic host cells are provided that display proteins on the surface of the cell via interaction of protein-PDZ-binding peptide fusions to PDZ Domain-cell surface protein fusions.

One aspect of the disclosure provides a polynucleotide (e.g., DNA, cDNA, RNA) encoding a cell surface protein fused to a PDZ Domain and/or a polynucleotide encoding a protein of interest (such as a polypeptide binding agent, an antibody, or antigen-binding fragment thereof), fused to a PDZ-binding peptide. In some embodiments, the polynucleotides are in the same vector; in other embodiments they are in different vectors; and in yet other embodiments one polynucleotide, e.g., the polynucleotide encoding a cell surface protein fused to a PDZ Domain, is integrated into the host cell genome.

Related aspects of the disclosure provide these polynucleotides operably linked to sequences that regulate expression of the encoded fusion protein(s), and vectors or chromosomes comprising these polynucleotides. Another related aspect of the disclosure provides host cells comprising such polynucleotides and/or vectors, and methods of using such host cells to display the protein of interest on the host cell surface. Yet another related aspect of the disclosure provides the fusion proteins encoded by the polynucleotides, either displayed on the surface of a host cell, or in an isolated or purified form. In particular, isolated or purified antibodies retaining the PDZ-binding peptide portion are contemplated.

In some or any of the embodiments described herein, the PDZ-binding peptide is 5 to 20 or 5 to 15 amino acids in length, for example 5, 6, 7, 8, 9, 10, 11, 12, 13, 14, 15, 16, 17, 18, 19, or 20 amino acids in length or any range between any of these lengths. In example embodiments, the PDZ-binding peptide is 15 or fewer amino acids in length, or 14, 13, 12, 11, 10, 9, 8, 7, 6, or 5 or fewer amino acids in length.

In some or any of the embodiments herein, the PDZ-binding peptide comprises a C-terminal sequence of NorpA (SEQ ID NO: 1) or is a peptide at least 80%, 85% or 90% identical to a fragment thereof at least 7 amino acids in length.

In an exemplary embodiment, the PDZ-binding peptide sequence is GKTEFCA (the last 7 amino acid residues of SEQ ID NO: 1).

In some or any of the embodiments herein, the PDZ-binding peptide is fused to the C-terminus of the protein of interest, e.g., antibody or antigen-binding fragment thereof.

In some or any of the embodiments herein, the PDZ Domain is about 80 to 120 amino acids in length, for example 80, 81, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 amino acids in length, or any range between any of these lengths.

In example embodiments of the disclosure, the PDZ Domain is selected from the group consisting of an InaD PDZ domain (SEQ ID NO: 2), a Dishevelled 1-like (DVL1L) PDZ domain (SEQ ID NO: 3), a proTGF-alpha cytoplasmic domain-interacting proteins 18 (TACIP18) PDZ1 domain (SEQ ID NO: 4), a similar to TACIP18 (SITAC) PDZ1 domain (SEQ ID NO: 5), a PSD-95/SAP90 PDZ3 domain (SEQ ID NO: 6), an Erbin PDZ domain (SEQ ID NO: 7), a PDZ-like domain, a PDZ dimer, a tandem PDZ domain, or a fragment, an extension, or variant thereof. In example embodiments, the fragments are at least about 20, 25, 30, 35, 40, 45, 50, 55, 60, 65, 70, 75, or 80 amino acids in length. In example embodiments, the extension is at least about 5, 6, 7, 8, 9, 10, 11, 12, 13, 14, 15, about 20, about 25, or about 30 amino acids in length. In example embodiments, the extension comprises residues 394-399 of SEQ ID NO: 6. In example embodiments, the variants comprise an amino acid sequence at least 80%, 85%, 90% or 95% identical to at least 50 amino acids of such domains. In some or any of the embodiments herein, the PDZ Domain is an InaD PDZ1 Domain as defined herein.

In some or any of the embodiments herein, the polynucleotide encoding a cell surface protein fused to a PDZ Domain further encodes an enhancer domain. In example embodiments, the enhancer domain is a variant of the 10^{th} fibronectin type III domain of human fibronectin (FN3), for example, an amino acid sequence at least 80%, 85%, 90% or 95% identical to at least 50 amino acids of FN3.

In some or any of the embodiments herein, the polynucleotide encoding a cell surface protein fused to a PDZ Domain and/or the polynucleotide encoding a protein of interest (e.g., an antibody, or antigen-binding fragment thereof), fused to a PDZ-binding peptide further encodes a fluorescent marker protein.

In some or any of the embodiments herein, the host cell is selected from the group consisting of a eukaryotic cell and a prokaryotic cell. In some or any of the embodiments herein, the eukaryotic cell is a yeast cell or a mammalian cell.

In example embodiments, the yeast cell is selected from the group consisting of S. cerevisiae, P. pastoris, C. albicans, H. polymorpha, Y. lipolitica, and S. pombe.

In example embodiments, the prokaryotic cell is selected from the group consisting of Escherichia coli, Salmonella typhimurium, Bacillus subtilis, Pseudomonas aeruginosa, and Serratia marcescans.

In example embodiments, the mammalian cell is selected from the group consisting of CHO cells, COS-7 cells, human embryonic kidney line (293, or variants thereof, e.g., 293E, 293T, or 293 cells subcloned for growth in suspension culture), BHK cells, TM4 cells, CV1 cells, VERO-76 cells, HeLa cells, MDCK cells, BRL 3A cells, W138 cells, Hep G2 cells, MMT cells, TR1 cells, MRC 5 cells, FS4 cells, and Hep G2 cells.

In example embodiments, when the host cell is a yeast cell, the cell surface protein is a cell wall protein, for example, Aga1, Aga2, Aga1, Cwp1, Cwp2, Gas1p, Yap3p, Flo1p, Crh2p, Pir1, Pir2, Pir3, or Pir4, or a fragment or variant of any of these proteins.

In example embodiments, when the host cell is a prokaryotic cell, the cell surface protein is an outer membrane protein, for example, FliC, pullulunase, OprF, OprI, PhoE, MisL, or cytolysin, or a fragment or variant of any of these proteins.

In example embodiments, when the host cell is a mammalian cell, the cell surface protein comprises any suitable transmembrane domain of any known cell membrane proteins, or a polypeptide with a GPI anchor sequence, or a fragment or variant thereof, or a non-cleavable type II signal anchor sequence.

In example embodiments, the fragments of such cell wall proteins are at least about 20, 25, 30, 35, 40, 45, 50, 60, 70, 80, 90, 100, 120, 140, 160, 180, or 200 amino acids in length. In example embodiments, the variants thereof comprise an amino acid sequence at least 80%, 85%, 90% or 95% identical to at least 100 amino acids of such domains.

In some or any of the embodiments herein, the antibody is a tetrameric IgG immunoglobulin comprising two heavy chains and two light chains.

In some or any of the embodiments herein, the antigen-binding fragment of the antibody comprises at least the heavy chain variable region and/or the light chain variable region. In example embodiments, the antigen-binding fragment of the antibody comprises a Fab, or an scFv.

In some or any of the embodiments herein, the polynucleotide encoding a cell surface protein fused to a PDZ Domain further comprises a signal sequence directing the cell surface protein to the cell surface.

In example embodiments, the signal sequence is an Aga2 signal sequence when the host cell is a yeast cell. In various embodiments, the signal sequence is derived from Mating Factor α1 (MFα1), Invertase (SUC2), Acid phosphatase (PHOS), Beta glucanase (BGL2), Inulinase (INU1A), AGA1, AGα1, FLO1, GAS1, CWP1, or CWP2, or a fragment or variant thereof.

In some or any of the embodiments herein, the PDZ Domain-PDZ binding peptide interaction has a K_{d} of about 100 nM or less (where a lower number indicates stronger binding affinity). In various embodiments, the PDZ Domain-PDZ binding peptide interaction has a K_{d} of about 100 nM or less, about 120 nM or less, about 140 nM or less, about 160 nM or less, about 180 nM or less, about 200 nM or less, about 240 nM or less, about 280 nM or less, about 300 nM or less, about 350 nM or less, about 400 nM or less, about 450 nM or less, about 500 nM or less, about 600 nM or less, about 700 nM or less, about 800 nM or less, about 900 nM or less, about 1 μM or less, about 10 μM or less, about 100 μM or less, or about 500 μM or less.

The polynucleotides of the disclosure may be operably linked to promoters, enhancers or one or more other transcriptional regulatory sequences, optionally as part of a vector comprising these sequences. Host cells comprising such polynucleotides or vectors may be prepared using methods known in the art or described herein.

Methods of using such host cells to display the protein of interest on the host cell surface may involve culturing the host cells for a time and under conditions that permit the expression of the encoded fusion proteins and linkage of the fusion proteins in a manner to display the protein of interest on the cell surface.

In another aspect, the invention contemplates a plurality of cells comprising at least 10̂3, at least 10̂4, at least 10̂5, at least 10̂6, at least 10̂7, at least 10̂8, at least 10̂9, or at least 10̂10 different eukaryotic host cells according to any of the preceding embodiments, each such eukaryotic host cell expressing on its surface a different protein of interest (e.g., polypeptide binding agent, or antibody, or antigen-binding fragment thereof).

In yet another aspect, the invention provides a method of displaying at least 10̂3, at least 10̂4, at least 10̂5, at least 10̂6, at least 10̂7, at least 10̂8, at least 10̂9, or at least 10̂10 different proteins of interest (e.g., polypeptide binding agents or antibodies, or antigen-binding fragments thereof), on cell surfaces, comprising culturing the plurality of cells described herein.

In some or any of the embodiments herein, the PDZ Domain and the PDZ-binding peptide interact and are linked by at least one disulfide bond. In a related embodiment, each of the PDZ Domain and the PDZ-binding peptide comprise a Cys residue that permits linkage by disulfide bonding. In some example embodiments, the Cys is a native amino acid, while in other example embodiments a native amino acid within the PDZ Domain and/or PDZ-binding peptide is replaced with a Cys. In some example embodiments, a Cys residue is located at the −1 position of the PDZ-binding peptide.

In a related aspect, the disclosure provides methods of using the plurality of host cells expressing different proteins of interest, involving screening for one or many proteins of interest that bind to an antigen.

In some or any of the embodiments herein, the method further comprises contacting the plurality of cells with an antigen. In another embodiment, the method further comprises selecting cells which bind to the antigen.

In some or any of the embodiments of the method, the selection is through fluorescence-activated cell sorting (FACS), bead-based sorting, or solid phase panning. In a related embodiment, the bead-sorting is magnetic-activated cell sorting (MACS). Methods of carrying out the selection are described in greater detail in the detailed description.

In another aspect, there is provided a method of selecting an antibody, or antigen-binding fragment thereof, comprising: (a) contacting a plurality of phage displaying antibody or antigen-binding fragments with an antigen, and selecting phage which bind to the antigen, and (b) contacting the plurality of yeast cells displaying an antibody or antigen binding fragment thereof with said antigen, and selecting cells which bind to the antigen.

In another aspect, there is provided a method of selecting an antibody, or antigen-binding fragment thereof, comprising: (a) contacting a plurality of phage displaying antibody or antigen-binding fragments with an antigen, and selecting phage which bind to the antigen, and (b) contacting the plurality of mammalian cells displaying an antibody or antigen binding fragment thereof with said antigen, and selecting cells which bind to the antigen.

In another aspect, there is provided a method of selecting an antibody, or antigen-binding fragment thereof, comprising: (a) contacting a plurality of yeast cells displaying an antibody or antigen binding fragment thereof with an antigen, and selecting cells which bind to the antigen, and (b) contacting the plurality of mammalian cells displaying an antibody or antigen binding fragment thereof with said antigen, and selecting cells which bind to the antigen. In some or any of the embodiments herein, the method further comprises the step of contacting a plurality of phage displaying antibody or antigen-binding fragments with an antigen, and selecting phage which bind to the antigen.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment,” “some embodiments,” “further embodiment,” “specific exemplary embodiments,” and/or “another embodiment,” each of these types of embodiments is a non-limiting example of a feature that is intended to be combined with any other feature, or combination of features, described herein without having to list every possible combination. Such features or combinations of features apply to any of the aspects of the invention. Similarly, where the disclosure describes polynucleotides encoding polypeptides characterized by certain features, polypeptides characterized by those features, host cells expressing such polypeptides, and all related methods of using such host cells are also contemplated by the disclosure. Where examples of values falling within ranges are disclosed, any of these examples are contemplated as possible endpoints of a range, any and all numeric values between such endpoints are contemplated, and any and all combinations of upper and lower endpoints are envisioned.

Numerous additional aspects and advantages of the invention will become apparent to those skilled in the art upon consideration of the following detailed description of the invention which describes presently preferred embodiments thereof. All U.S. patents, U.S. patent application publications, U.S. patent applications, foreign patents, foreign patent applications, and non-patent publications referred to in this application, are incorporated herein by reference, in their entireties.

### BRIEF DESCRIPTION OF THE DRAWINGS

FIG. 1 shows the yeast vector pTam15 in which DNA coding for XPA28 scFv is fused to DNA coding for the mature Aga2 protein (19-87).

FIG. 2 shows yeast vector pTam16 in which the first PDZ domain of InaD (amino acids 11-107 of SEQ ID NO: 3) (InaD PDZ1) is fused to Aga2.

FIG. 3 shows the yeast vector pTam28 in which DNA coding for XPA28 scFv is fused to DNA coding for the C-terminal seven residues of NorpA (amino acids 1089-1095 of SEQ ID NO: 1) (NorpA tether). Included also in the vector is DNA coding for the InaD PDZ1/Aga2 fusion protein. Both proteins are expressed concurrently using identical GAL1 promoters.

FIG. 4 shows flow cytometric analysis of yeast cells transformed with pTam28 (A), pTam15 (B) and pTam16 (C). Induced cells were incubated with biotinylated IL-1β and a c-Myc antibody. Bivariate plots of PE and Alexa Fluor 647 fluorescence show the correlation between antigen binding and scFv expression. The number of cells in each quadrant is shown as a percentage of total.

FIG. 5 shows dose-dependent binding of IL-β by yeast cells transformed with pTam15 and pTam28. The K_{D} was determined by a plot of the mean PE fluorescence (percentage of total) against IL-1β concentration.

FIG. 6 shows the yeast vector pTam32 in which DNA coding for XPA28 scFv is fused to DNA coding for the mature Aga1 protein (amino acids 23-725 of SEQ ID NO: 8).

FIG. 7 shows bivariate plots of IL-1β binding and c-Myc staining of yeast cells transformed with pTam15 (A) and 32 (B) as measured by PE and Alexa Fluor 647 fluorescence. The number of cells in each quadrant is shown as percentage of total.

FIG. 8 shows the yeast vector pTam34 in which XPA28 scFv is expressed with the NorpA tether and the DNA coding for InaD PDZ1 is fused to DNA coding for the Aga1 protein.

FIG. 9 shows bivariate plots of IL-1β binding and c-Myc staining of yeast cells transformed with pTam28 (A) and 34 (B) as measured by PE and Alexa Fluor 647 fluorescence. The number of cells in each quadrant is shown as a percentage of total.

FIG. 10 shows the yeast vector pTam35 which is similar to the parental vector pTam34 with the exception that the detection tag on the InaD PDZ1/Aga1 fusion has been changed from c-Myc to HA epitope.

FIG. 11 shows the IL-1β binding, c-Myc and HA staining properties of cells transformed with pTam35 as measured by PE (A), Alexa Fluor 647 (B) and Alexa Fluor 488 (C) fluorescence respectively. Both uninduced (grey fill) and induced (no fill) cells are shown.

FIG. 12 shows the yeast vector pTam37 which is similar to the parental vector pTam35 with the exception that the c-Myc epitope now precedes the His6 tag at the C-terminus of XPA28 scFv.

FIG. 13 shows the IL-1β binding, c-Myc and HA staining properties of cells transformed with pTam37 as measured by PE (A), Alexa Fluor 647 (B) and Alexa Fluor 488 (C) fluorescence respectively. Both uninduced (grey fill) and induced (no fill) cells are shown.

FIG. 14, Panel A shows the mammalian vector pXIBM14 for expression of XPA28 IgG using a single promoter and IRES2 preceding the light and heavy chain respectively. Secreted XPA IgG was purified by Protein A Sepharose and analyzed by reducing SDS-PAGE (B).

FIG. 15, Panel A shows a series of mammalian vectors pXIBM32, 34, and 36 in which the NorpA tether has been fused to the C-terminus of the IgG1 heavy chain with either no amino acids, three amino acid (GAA), or five amino acid (GGGGS) spacer, respectively. Panel B shows the mammalian vector pTam29 in which the InaD PDZ1 is fused to the transmembrane domain of PDGFR β (amino acid residues 513-561 of SEQ ID NO: 9).

FIG. 16 shows flow cytometric analysis of HEK293 cells transfected with pXIBM14 alone (A), pTam29 alone (B), pXIBM32 and pTam29 (C), pXIBM34 and pTam29 (D), and pXIBM36 and pTam29 (E). Cells were incubated with biotinylated IL-1β and stained with a c-Myc antibody. Bivariate plots of PE and Alexa Fluor 647 fluorescence shows the correlation between antigen binding and InaD PDZ1 expression. The number of cells in each quadrant is shown as a percentage of total.

FIG. 17 shows reducing SDS-PAGE analysis of purified XPA IgG from cells transfected with pXIBM14 (− NorpA tether) and pXIBM32 (+ NorpA tether).

FIG. 18 shows the modifications to pTam37 resulting in the following vectors: (1) pTam49 which contains the C1094S mutation in the NorpA tether; (2) pTam50 which contains the C31S mutation in InaD PDZ1; (3) pTam51 which contains a TGATGA insertion between the Aga2 signal sequence and InaD PDZ1; and (4) pTam52 which contains a TGATGA insertion between Aga2 signal sequence and XPA28 scFv.

FIG. 19 shows the flow cytometric analysis of BJ5465 cells transformed with pTam37 (A), pTam49 (B), pTam50 (C), pTam51 (D), and pTam52 (E). Cells were incubated with biotinylated IL-1β and stained with a HA antibody. Bivariate plots of PE and Alexa Fluor® 488 fluorescence show the correlation between antigen binding and InaD PDZ1 expression.

FIG. 20 shows four successive rounds (A-D) of library enrichment for transferrin binders using FACS. Cells were incubated with biotinylated transferrin and stained with a HA antibody. Bivariate plots of PE and Alexa Fluor 488 fluorescence show the correlation between antigen binding and InaD PDZ1 expression. Sorting gates used during FACS are indicated and the number of collected cells is shown as a percentage of parent population.

FIG. 21 shows an estimation of affinity for transferrin for three scFv clones isolated after four rounds of library enrichment. The mean PE fluorescence is plotted against transferrin concentration in order to derive the estimated K_{D}, which is shown.

FIG. 22 shows the vector pTam48.

FIG. 23 shows the vector pVV47, which displays anti-IL-1β Fab.

FIG. 24 shows the vector pVV42, which displays anti-IL-1β IgG.

FIG. 25 shows flow cytometric analysis of yeast cells transformed with different anti-IL-1β fragments: pTam37 (scFv), pVV47 (Fab) and pVV42 (IgG). In each panel, >80% of galactose-induced cells are positive for both anti-HA antibody (the detection tag on the InaD PDZ1/Aga1 fusion) and biotin-IL-1β.

FIG. 26 shows flow cytometric analysis of transfected HEK293E cells (A) before and (B) after magnetic cell separation. Cells were transfected with DNA corresponding to IgGs enriched for Tie2 binding following three rounds of phage panning. Bivariate plots of PE and Alexa Fluor® 647 shows the correlation between Tie2 binding and InaD PDZ1 expression. The number of cells in each quadrant is shown as a percentage of the total.

FIG. 27 shows relative levels of AKT phosphorylation at serine 473 of CHOK1-Tie2 cells treated with Ang1 and ten anti-Tie2 IgGs (A3, A10, A11, B1, B4, B6, B8, B12, C3, and C4). Anti-KLH treated and untreated cells were included as negative controls. Also shown are dissociation constants for several of the IgGs for soluble Tie2, as determined by Biacore.

FIG. 28 illustrates an IgG yeast display library constructed from round 3 output from phage Fab library and panned against Tie-2. FACS of the yeast library isolated three populations of cells double positive for antigen binding (detected by biotinylated Tie-2-Fc labeled with streptavidin-PE) and antibody display (detected by APC-tagged anti-lambda).

### DETAILED DESCRIPTION

This invention relates to materials and methods useful for displaying proteins of interest, including antibodies, on the surface of a cell. Both prokaryotic and eukaryotic cells capable of displaying proteins on the cell surface are provided. The methods and materials provided in this disclosure relate to the interaction between a fusion of the protein of interest to a PDZ-binding peptide and a fusion of a PDZ Domain to a cell surface protein, to display a protein of interest on the surface of a cell. One advantageous aspect of the invention is that the small size of the PDZ-binding peptides causes less potential interference with folding and solubility of the proteins of interest, particularly when the protein of interest is multimeric and may comprise more than one different polypeptide chain. For example, there will be less interference with antibody assembly and binding to antigen, and fusion proteins comprising antibodies or antigen-binding fragments thereof with PDZ-binding peptides are easily isolated or purified and tested separately (not in association with the host cell) for binding to antigen. The examples herein show that the materials and methods of the disclosure permit tetrameric immunoglobulins comprising two heavy chains and two light chains to be expressed on the cell surface.

Another potential advantage is the ability to rely on fluorescent-activated cell sorting techniques to enrich and segregate cells that exhibit strong binding properties, which permits identification of rarer clones expressing candidate proteins of interest, e.g., antibodies, such as candidates occurring at frequencies below 10⁻⁶. Another potential advantage is the ability to display different proteins of interest on the same cell. For example, different proteins of interest may be cloned, each with a NorpA tether, and expressed with a single copy of an InaD PDZ1 domain/Aga 1 fusion. Yet another potential advantage of the present invention compared to other techniques based on linkage to cell surface proteins is the ability to prepare relatively large libraries with increased diversity.

### A. DEFINITIONS

As used herein, an antibody that “specifically binds” is “antigen specific”, is “specific for” antigen or is “immunoreactive” with an antigen refers to an antibody or polypeptide binding agent of the invention that binds an antigen with greater affinity than other antigens of unrelated to similar sequence, preferably at least 10³, 10⁴, 10⁵, or 10⁶ greater affinity. In one aspect, the antibody or polypeptide binding agents of the invention, or fragments, variants, or derivatives thereof, will bind with a greater affinity to human antigen as compared to its binding affinity to similar antigens of other, i.e., non-human, species, but polypeptide binding agents that recognize and bind orthologs of the target are contemplated.

For example, a polypeptide binding agent that is an antibody or fragment thereof “specific for” its cognate antigen indicates that the variable regions of the antibodies recognize and bind the desired antigen with a detectable preference (e.g., where the desired antigen is a polypeptide, the variable regions of the antibodies are able to distinguish the antigen polypeptide from other known polypeptides of the same family, by virtue of measurable differences in binding affinity, despite the possible existence of localized sequence identity, homology, or similarity between family members). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of a polypeptide binding agent, e.g. antibody, for use in the methods of the invention are well known and routinely practiced in the art. For a comprehensive discussion of such assays, see Harlow et al. (Eds), Antibodies: A Laboratory Manual; Cold Spring Harbor Laboratory; Cold Spring Harbor, N.Y. (1988), Chapter 6. Antibodies for use in the invention can be produced using any method known in the art and described in greater detail herein.

The term “epitope” refers to that portion of any molecule capable of being recognized by and bound by a selective binding agent at one or more of the antigen binding regions. Epitopes usually consist of chemically active surface groupings of molecules, such as, amino acids or carbohydrate side chains, and have specific three-dimensional structural characteristics as well as specific charge characteristics. Epitopes as used herein may be contiguous or non-contiguous.

The term “derivative” when used in connection with polypeptides (e.g., proteins of interest, polypeptide binding agents or antibodies or antigen-binding fragments thereof) refers to polypeptides chemically modified by such techniques as ubiquitination, glycosylation, deglycosylation, conjugation to therapeutic or diagnostic agents, labeling (e.g., with radionuclides or various enzymes), covalent polymer attachment such as pegylation (derivatization with polyethylene glycol) and insertion or substitution by chemical synthesis of amino acids such as ornithine, which do not normally occur in human proteins. Derivatives retain the binding properties of underivatized molecules of the invention.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavadin, dioxigenin, haptens and proteins for which antisera or monoclonal antibodies are available (e.g., c-myc, HA), or nucleic acid molecules with a sequence complementary to another labeled nucleic acid molecule. The detectable moiety often generates a measurable signal, such as a radioactive, chromogenic, or fluorescent signal, that can be used to quantitate the amount of bound detectable moiety in a sample.

The term “host cell” is understood to refer not only to the particular subject cell or cells but also the progeny thereof. It is also understood that, during culture, natural or accidental mutations may occur in succeeding generations and thus such progeny may not be completely identical to the parent cell, but are still included within the scope of the term as used herein.

The term “operably linked” refers to a functional relationship between two or more polynucleotide (e.g., DNA) segments. Typically, it refers to the functional relationship of a transcriptional regulatory sequence to a transcribed sequence. For example, a promoter/enhancer sequence of the invention, including any combination of cis-acting transcriptional control elements, is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance. A polylinker provides a convenient location for inserting coding sequences so the genes are operably linked to a promoter. Polylinkers are polynucleotide sequences that comprise a series of three or more closely spaced restriction endonuclease recognition sequences.

The term “signal sequence” refers to a polynucleotide sequence which encodes a short amino acid sequence (i.e., signal peptide) present at the NH₂-terminus of certain proteins that are normally exported by cells to noncytoplasmic locations (e.g., secretion) or to be membrane components. Signal peptides direct the transport of proteins from the cytoplasm to noncytoplasmic locations.

As used herein “binding” is the physical association between two or more distinct molecular entities that results from a specific network of non-covalent interactions consisting of one or more of the weak forces including hydrogen bonds, Van der Waals, ion-dipole and hydrophobic interactions and the strong force ionic bonds. The level or degree of binding may be measured in terms of affinity. Affinity, or “binding affinity”, is a measure of the strength of the binding interaction between two or more distinct molecular entities that can be defined by equilibrium binding constants or kinetic binding rate parameters. Examples of suitable constants or parameters and their measurement units are well known in the art and include but are not limited to equilibrium association constant (K_{A}), e.g. about 10⁵M⁻¹ or higher, about 10⁶M⁻¹ or higher, about 10⁷M⁻¹ or higher, about 10⁸M⁻¹ or higher, about 10⁹M⁻¹ or higher, about 10¹⁰M⁻¹ or higher, about 10¹¹ M⁻¹ or higher or about 10¹²M⁻¹ or higher; equilibrium dissociation constant (K_{D}), e.g., about 10⁻⁵M or less, or about 10⁻⁶M or less, or about 10⁻⁷M or less, or about 10⁻⁸M or less, or about 10⁻⁹M or less, or about 10⁻¹⁰M or less, or about 10⁻¹¹M or less, or about 10⁻¹²M or less; on-rate (e.g., sec⁻¹, mol⁻¹) and off-rate (e.g., sec⁻¹)). In the case of K_{A}, higher values mean “stronger” or “strengthened” or “greater” binding affinity while in the case of K_{D}, lower values mean “stronger” or “strengthened” or “greater” binding affinity. As used herein, a “strengthened” binding rate parameter means increased residency time, faster association or slower dissociation. As used herein, a “weakened” binding rate parameter means decreased residency time, slower association or faster dissociation.

Affinity between two compounds, e.g. between an antibody and an antigen, may be measured directly or indirectly. Indirect measurement of affinity may be performed using surrogate properties that are indicative of, and/or proportional to, affinity. Such surrogate properties include: the quantity or level of binding of a first component to a second component, or a biophysical characteristic of the first component or the second component that is predictive of or correlated to the apparent binding affinity of the first component for the second component. Specific examples include measuring the quantity or level of binding of first component to a second component at a subsaturating concentration of either the first or the second component. Other biophysical characteristics that can be measured include, but are not limited to, the net molecular charge, rotational activity, diffusion rate, melting temperature, electrostatic steering, or conformation of one or both of the first and second components. Yet other biophysical characteristics that can be measured include determining stability of a binding interaction to the impact of varying temperature, pH, or ionic strength.

Measured affinity is dependent on the exact conditions used to make the measurement including, among many other factors, concentration of binding components, assay setup, valence of binding components, buffer composition, pH, ionic strength and temperature as well as additional components added to the binding reaction such as allosteric modulators and regulators. Quantitative and qualitative methods may be used to measure both the absolute and relative strength of binding interactions.

### B. PDZ DOMAINS AND PDZ-BINDING PEPTIDES

1. PDZ Domains

The present invention provides methods and cells useful for displaying proteins, including antibodies and antibody fragments, on the surface of cells using fusion proteins comprising a cell surface protein fused to a PDZ Domain. PDZ domains were originally described as containing conserved structural elements among the 95 kDa post-synaptic density protein (PSD-95), the Drosophila tumor suppressor discs-large (dlg), and the tight junction protein zonula occludens-1 (ZO-1). These domains are found in a large and diverse set of proteins. They generally bind to short carboxyl-terminal peptide sequences located on the carboxyl-terminal end of interacting proteins, but may also bind to internal sequences.

PDZ domains are generally composed of a 5- to 6-stranded anti-parallel β-barrel and 2-3 α-helices. Helix α2 and strand β2 form either side of the conserved peptide binding cleft within the PDZ domain fold. The loop between the β1 and β2 strands forms the C-terminal carboxylate binding loop. C-terminal peptides (e.g., PDZ-binding peptides) bind as an antiparallel β strand in a groove formed by helix α2 and strand β2. The conserved Gly-Leu-Gly-Phe (GLGF) sequence of the PDZ domain is found within the β1 and β2 connecting loop and is important for hydrogen bond coordination of the C-terminal carboxylate group. The N- and C-termini of the PDZ domain are located near each other on the opposite side of the PDZ domain from the peptide-binding site.

Hung and Sheng (J. Biol. Chem., 277:8, 5699-5702 (2002)) classified PDZ domains into three classes based on binding specificity for their peptide ligands. The binding specificity of PDZ domains is generally determined by the interaction of the first residue of helix α2 and the side chain of the −2 residue of the C-terminal PDZ-binding ligand (numbering based on C-terminal amino acid being the “0” position). In Class I PDZ interactions, such as those of PSD-95, a serine or threonine residue occupies the −2 position of the PDZ-binding ligand. The side chain hydroxyl group forms a hydrogen bond with the N-3 nitrogen of a histidine residue at position α2-1 (the first residue of the second alpha helix, a2), which is highly conserved among Class I PDZ domains. In contrast, class II PDZ interactions are characterized by hydrophobic residues at both the −2 position of the PDZ-binding peptide ligand and the α2-1 position of the PDZ domain. A third class of PDZ domains, such as in neuronal nitric-oxide synthase (nNOS), prefers negatively charged amino acids at the −2 position of the PDZ-binding ligand. This specificity is determined by the coordination of the hydroxyl group of a tyrosine residue at position α2-1 with the side chain carboxylate of the −2 residue of the PDZ-binding ligand. PDZ domains generally interact with the C-terminal 3-4 amino acids of their protein targets, including the free carboxylate group (Hillier et al., (1999) Science 284: 812-815). Type I PDZ domains bind to the consensus sequence S/T-X-V/L, where X is any residue (Doyle et al., (1996) Cell 85: 1067-1076; Songyang et al., (1997) Science 275: 73-77), while type II PDZ domains bind to the more general sequence Φ-X-Φ, where Φ is usually a large, hydrophobic residue (Daniels et al., (1998) Nat. Struct. Biol. 5: 317-325).

PDZ domain classification has been extended beyond the three classes described above using sequence- and structure-based information, allowing improved prediction of PDZ domain specificity and design of novel PDZ domain/peptide interactions (Tonikian et al., (2008) PLos Biol 6: e239; Kaufmann et al., J. Mol. Model. (2011) 17: 315-324).

In contrast to the majority of PDZ domains, some PDZ domains interact with internal peptide sequences. For example, the PDZ domain of PSD-95 interacts with an internal region of nNOS. In the crystal structure of the nNOS-PSD-95 PDZ complex, amino acid residues adjacent to the canonical PDZ domain of nNOS form a two-stranded β-hairpin “finger,” which docks in the peptide-binding groove of the PSD-95 PDZ domain. The sharp β turn of the β-finger binds to the same site as the terminal carboxylate group of peptide ligands. PDZ domains that bind internal peptides (i.e., peptides not at the C-terminus) are considered within the scope of the present invention.

As used herein, the term “PDZ Domain” refers to a domain of a protein that comprises one or more of these conserved structural elements described above characteristic of PDZ domains, e.g., the helix α2 and strand β2 which form the conserved peptide binding cleft, the loop between the β1 and β2 strands which forms the C-terminal carboxylate binding loop, the GLGF repeat, the N-3 containing histidine residue at position 1 of helix α2 (or conservative substitution thereof that contains a suitable nitrogen) which is highly conserved among Class I PDZ domains, the hydrophobic residues at position 1 of helix α2, which is highly conserved among Class II PDZ domains, and/or the hydroxyl-containing tyrosine residue at position 1 of helix α2 (or conservative substitution thereof that contains an hydroxyl), which is highly conserved among Class III PDZ domains, as well as fragments, extensions, or variants thereof. In example embodiments, the fragments are at least about 20, 25, 30, 35, 40, 45, 50, 55, 60, 65, 70, 75, 80 or more amino acids in length. In example embodiments, the extension is at least about 5, 6, 7, 8, 9, 10, 11, 12, 13, 14, 15, about 20, about 25, or about 30 amino acids in length. In example embodiments, the extension comprises residues 394-399 of SEQ ID NO: 6. In example embodiments, the variants comprise an amino acid sequence at least 80%, 85%, 90% or 95% identical to at least 50 amino acids of such domains, and preferably one or more of the conserved elements identified above is retained. For example, the helix α2 and strand β2 which form the conserved peptide binding cleft are retained, and optionally the GLGF repeat, the N-3 containing histidine residue at position 1 of helix α2 (or conservative substitution thereof that contains a suitable nitrogen) which is highly conserved among Class I PDZ domains, the hydrophobic residues at position 1 of helix α2, which is highly conserved among Class II PDZ domains, and/or the hydroxyl-containing tyrosine residue at position 1 of helix α2 (or conservative substitution thereof that contains an hydroxyl), which is highly conserved among Class III PDZ domains, is (are) also retained. The term “PDZ Domain” includes but is not limited to a PDZ domain of a post synaptic density 95 (PSD-95) (SEQ ID NO: 10), tumor suppressor discs-large (dlg) (SEQ ID NO: 11), tight junction protein zonular occludens (ZO-1) (SEQ ID NO: 12), InaD (SEQ ID NO: 2), a Dishevelled 1-like (DVL1L) (SEQ ID NO: 3), a proTGF-alpha cytoplasmic domain-interacting proteins 18 (TACIP18) (SEQ ID NO: 4), a similar to TACIP18 (SITAC) (SEQ ID NO: 5), a PDZ-like domain, a PDZ dimer, a tandem PDZ domain (Lee & Zheng, Cell Communication and Signaling 2010 8: 8), a PSD-95/SAP90 PDZ3 domain (SEQ ID NO: 6), and an Erbin (SEQ ID NO: 7), or fragments, extensions (Petit et al., PNAS 106: 18249-54 (2009) and Wang et al., Protein Cell 1: 737-51 (2010)), or variants thereof that can associate with a PDZ binding peptide described herein. In any of these embodiments, a PDZ domain (e.g., PDZ1 of InaD or TACIP18 or SITAC) which naturally comprise a Cys are contemplated. The term “PDZ Domain” also includes vertebrate homologs of PDZ1 family members, including, but not limited to mammalian and avian homologs. Representative mammalian homologs of PDZ domain family members include, but are not limited to murine and human homologs, or invertebrate proteins, such as from Drosophila melanogaster. In example embodiments, the fragments of the PDZ domains included within the term “PDZ Domains” are at least about 20, 25, 30, 35, 40, 45, 50, 55, 60, 65, 70, 75, 80 or more amino acids in length. In example embodiments, the variants included within the term “PDZ Domains” comprise an amino acid sequence at least 80%, 85%, 90% or 95% identical to at least 50 amino acids of such PDZ domains. In some embodiments, one or more of the conserved elements identified above is retained.

PDZ1 domain from Inactivation no after-potential D (InaD), which shares the general PDZ domain topology, is set forth as amino acids 11 through 107 of SEQ ID NO: 2. InaD is a critical protein in the Drosophila phototransduction pathway, a well-characterized G protein-coupled, phospholipase C-mediated signaling cascade (Scott & Zuker, (1998) Nature 395: 805-808; Xu et al., (1998) J. Cell Biol. 142: 545-555; Scott et al., (1995) Neuron 15: 919-927). InaD is composed nearly completely of five PDZ domains (van Huizen et al., (1998) EMBO J. 17: 2285-2297; Tsunoda et al., (1997) Nature 388: 243-249; Shieh et al., (1997) Proc. Natl. Acad. Sci. U.S.A. 94: 12682-12687), so named for the first three proteins in which this domain was characterized: Post-synaptic density 95, Discs-large, and Zonular occludens (Kennedy, (1995) Trends Biochem Sci 20: 350; Morais Cabral et al., (1996) Nature 382: 649-652; Doyle et al., (1996) Cell 85: 1067-1076). Each of the PDZ domains of InaD has been implicated in binding one or more of the proteins involved in phototransduction, bringing the complex together in the proper cellular location for efficient signaling (Tsunoda et al., (1997) Nature 388: 243-249; Wes et al., (1999) Nat Neurosci 2: 447-453; Montell, (1999) Annu Rev Cell Dev Biol 15: 231-268; Fanning & Anderson, (1999) Curr. Opin. Cell Biol. 11: 432-439).

The InaD protein of Drosophila comprises 674 amino acids (SEQ ID NO: 2), has a molecular weight of 74,332 daltons and comprises five PDZ domains. These five PDZ domains form the majority of the protein's structure. The domains are numbered PDZ1 through PDZ5. PDZ1, the N-terminal domain of InaD, comprises residues 11-107 of the InaD protein. In the disclosure presented herein PDZ1 is referred to specifically in some embodiments; however, the disclosure and discussion of embodiments, methods, and techniques can also be applied to another PDZ domain, such as PDZ2, PDZ3, PDZ4, and PDZ5.

The PDZ1 domain of InaD is known to bind the C-terminus of NorpA (SEQ ID NO: 1). This interaction is mediated by a disulfide bond formed between these two proteins. The disulfide bond is formed between Cys(−1) of NorpA (numbering based on C-terminal amino acid being the “0” position) and Cys 31 of the InaD PDZ1.

In one embodiment a PDZ Domain (e.g., a PDZ1 domain) useful according to the present invention is derived from the InaD protein found in Drosophila, i.e., “InaD PDZ1 Domain”, and is fused to a cell surface protein. “InaD PDZ1 Domain” includes fragments of the InaD PDZ1 domain (amino acids 11-107) that are at least about 20, 25, 30, 35, 40, 45, 50, 55, 60, 65, 70, 75, 80 or more amino acids in length, and variants thereof that comprise an amino acid sequence at least 80%, 85%, 90% or 95% identical to at least 50 amino acids of the InaD PDZ1 domain. In some embodiments, one or more of the conserved elements identified above is retained, It is contemplated that a PDZ Domain (e.g., a PDZ1 domain) of the present invention is derived from any species, including but not limited to, Drosophila melanogaster, Caenorhabditis elegans, Calliphora vicina, Homo sapiens, Mus musculus, and any other species having PDZ domains.

In one embodiment, the PDZ Domain comprises a Cys residue in the peptide-binding cleft. In one embodiment, the PDZ Domain comprising a Cys residue is a Dishevelled 1-like (DVL1L) PDZ. In another embodiment, the PDZ Domain comprising a Cys residue is a proTGF-alpha cytoplasmic domain-interacting proteins 18 (TACIP18) PDZ1. In another embodiment, the PDZ Domain comprising a Cys residue is a similar to TACIP18 (SITAC) PDZ1.

In various embodiments, the PDZ Domain is about 80 to about 100 amino acids in length. In similar embodiments, the PDZ Domain is 80, 81, 81, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 amino acids in length, or any range between any of these endpoints.

In one embodiment, the PDZ Domain further comprises an enhancer domain. Huang et al. (Proc. Nat'l Acad. Sci. USA, 105:18, 6578-83 (2008); incorporated by reference it its entirety) described a system whereby PDZ domains could be engineered to generate binding sites with substantially improved binding affinity for native PDZ-binding peptides. The authors fused the 91 amino acid residue 10^{th} fibronectin type III domain of human fibronectin (FN3) to the 96 amino acid residue Erbin PDZ domain. The authors then constructed a phage-display library in which the three surface loops of FN3 were diversified. Several clones were identified exhibiting enhanced affinity to the ARVCF peptide. Such FN3 domains creating PDZ fusions with enhanced affinity for PDZ-binding peptides were termed “enhancer domains.” PDZ-FN3 fusions were termed “affinity clamps.” Thus, in some or any of the embodiments herein, the PDZ Domain is fused to an enhancer domain, for example, an amino acid sequence at least 80%, 85%, 90% or 95% identical to at least 50 amino acids of FN3. Such affinity clamps and enhancer domains are contemplated for use in the present invention.

In various embodiments, the PDZ Domain is a tandem PDZ domain. Lee and Zheng (Cell Comm. & Signaling, 8:8 (2010); incorporated herein by reference in its entirety) described tandem arrangements of PDZ domains 1 and 2 from the GRIP-1 protein wherein each PDZ domain required the presence of the other for proper folding. Similarly, a tandem arrangement of the 4^{th} and 5^{th} PDZ domains from GRIP-1 was required for interaction with GluR2/3. Thus, in some or any of the embodiments herein, the PDZ Domain is a tandem PDZ domain, for example PDZ1 and PDZ2 from GRIP-1 (Accession #NP083012) or PDZ4 and PDZ5 from GRIP-1. Such tandem PDZ domains may comprise at least 2, 3, 4 or more PDZ domains of the same or different sequences.

In various embodiments, the PDZ Domain is a PDZ dimer comprising two PDZ domains (of the same or different sequence), that may be noncovalently or covalently bound, that retains the ability to bind to a PDZ-binding peptide.

In various embodiments, the PDZ Domain is a PDZ-like domain. Lee and Zheng (Cell Comm. & Signaling, 8:8 (2010)) described various proteins that adopt a PDZ-like fold consisting of 5 13-strands capped by 2-helices. Proteins with PDZ-like domains include HtrA (or DegP), DegS, and DegQ. Thus, in some or any of the embodiments herein, the PDZ Domain is a PDZ-like domain, a PDZ-like domain from HtrA, DegS, or DegQ.

2. PDZ-Binding Peptides

A PDZ-binding peptide useful according to the present invention can be of any length or sequence, although generally the portion that interacts with a PDZ domain is the C-terminal 3-4 amino acids of the PDZ-binding protein. As used herein, “PDZ-binding peptide” refers to an approximately 15- to 20-amino acid region at the C-terminus or surrounding the internal PDZ-binding region of a PDZ-binding protein; or fragments thereof that are at least 5, 6, 7, 8, 9, 10 or more amino acids in length; or variants of such fragments wherein 1, 2, 3, 4, 5, 6, 7, 8 or 9 substitutions, preferably conservative substitutions, are made to the native sequence, provided that a Cys residue is retained that permits disulfide linkage to the PDZ Domain.

In some or any embodiments herein, a PDZ-binding peptide is derived from the NorpA protein (SEQ ID NO: 1) (i.e., a NorpA PDZ-binding peptide), and is for example, derived from the 20 amino acids at the C-terminus of a NorpA protein (SEQ ID NO: 1). In some examples, the NorpA PDZ-binding peptide is a C-terminal fragment at least 4-20, or 5-15, or 5-20 amino acids in length, that may comprise one or more substitutions (e.g., 1, 2, 3, 4, 5, 6, 7, 8, or 9), preferably conservative substitutions, and that retains the Cys at the −1 position. In some or any embodiments, the peptide comprises the amino acid sequence X₁-X₂-X₃-C—X₄, where C is an invariant cysteine and X₁, X₂, X₃, and X₄ can be any residue (SEQ ID NO: 13). In alternative embodiments, these variable amino acids are as follows: X₁ is threonine, serine, or tyrosine; X₂ is glutamic acid or aspartic acid; X₃ is phenylalanine or tyrosine, and X₄ is alanine, glycine, leucine, isoleucine, or valine (SEQ ID NO: 14). In some or any embodiments, e.g., when the PDZ Domain interacting with the PDZ-binding peptide is a Type I PDZ domain, the PDZ-binding peptide comprises the consensus sequence S/T-X-V/L, where X is any residue. In some or any embodiments, e.g., when the PDZ Domain interacting with the PDZ-binding peptide is a Type II PDZ domain, the PDZ-binding peptide comprises the consensus sequence Φ-X-Φ, where Φ is a large, hydrophobic residue. A PDZ-binding peptide of the present invention can comprise any segment or fragment of a NorpA polypeptide (representative NorpA polypeptide set forth in SEQ ID NO: 1), or functional equivalent thereof as defined herein, so long as the segment, fragment, or functional equivalent thereof exhibits the functional characteristic of binding a PDZ1 domain polypeptide as defined herein. In a specific embodiment, the PDZ-binding peptide sequence is TEFCA (SEQ ID NO: 15), or a modified peptide wherein one, two, three, or four conservative substitutions are made, providing that the Cys residue is retained, preferably at position-1. In another embodiment, the PDZ-binding peptide sequence is GKTEFCA (SEQ ID NO: 16), or a modified peptide wherein one, two, three, four, five, or six conservative substitutions are made, providing that the Cys residue is retained, preferably at position −1. In another embodiment, the PDZ-binding peptide sequence is KTEFCA (SEQ ID NO: 17), or a modified peptide wherein one, two, three, four, or five conservative substitutions are made, providing that the Cys residue is retained, preferably at position −1.

As discussed above, the InaD PDZ1 domain binds the C-terminus of NorpA, which has a Cys residue at the −1 position of NorpA (i.e., the second-to-last residue of SEQ ID NO: 1). Additional examples of proteins with a naturally occurring Cys residue, e.g., at the −1 position, that are expected to interact with a cognate PDZ domain in a manner similar to the InaD-NorpA interaction include but are not limited to, the PDZ binding peptide from Drosophila Wingless (SwissProt accession No. P13217; C-terminal sequence TCL), Knirps (P10734; VCV), netrin A (Q24567; TCA); Human ZFP36 (17209; C-terminal sequence SCV), ZAP70 (P43403; ACA), Ulk-1 (O75385; ICA), adenylosuccinase (P30566; LCL), P53 induced protein 10 (O14682; FCL), NAG-2 (O14817; YCA), c-Myc (P01106; SCA), insulin-like peptide 4 (Q14641; LCT), glutathione peroxidase (P07203; SCA), 5-HT-2A (P28223; SCV), T-cadherin receptor (P55290; ACL), CD86 precursor (P42081; TCF), estradiol 17B hydrogenase (P56937; SCL), EGR-3 (Q06889; TCA), galactokinase I (P51570; LCL), and Frizzled 10 (trEMBL Q9ULW2; TCV). In some embodiments, the PDZ-binding peptide is from Rat hexokinase III (P27296; C-terminal sequence ACV), Olif. Rec. like prot I15 (P27296; FCL), Olif. Rec. like prot F3 (P23265; FCY), and D3 phosphoglycerate dehydrogenase (O08651; FCF). In any of these embodiments, the PDZ-binding peptide is a C-terminal fragment of any of the preceding proteins at least 4-20, or 5-15, or 5-20 amino acids in length, that may comprise one or more substitutions (e.g. 1, 2, 3, 4, 5, 6, 7, 8, or 9), preferably conservative substitutions, and that retains the Cys at the −1 position.

In some or any of the embodiments herein, the PDZ-binding peptide is 5 to 20 amino acids or 4 to 20 amino acids in length. In other embodiments, the PDZ-binding peptide is less than 15 amino acids in length, e.g., 3 to 15 amino acids in length. In various embodiments, the PDZ-binding peptide is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids in length, or any range between any of these endpoints.

In one embodiment, the PDZ-binding peptide is fused to the C-terminus of the protein of interest, e.g., polypeptide binding agent or the antibody or antigen-binding fragment thereof.

In some or any of the embodiments herein, a PDZ domain and PDZ-binding peptide pair is selected from Table 1. Exemplary PDZ domains and their respective ligands (i.e., PDZ-binding peptides) were obtained from PDZBase (Beuming et al., Bioinformatics, 21 (6): 827-828 (2005)) and are listed in Table 1.

TABLE 1
PDZ Domain/PDZ-binding peptide pairs

| PDZ | | | | PDZ | | | | | |
|---|---|---|---|---|---|---|---|---|---|
| containing | | SEQ | | Do- | | | | | SEQ |
| protein | Accession | ID | | main | | Accession | | C-terminal Sequence | ID |
| name | No. | NO | Species | No. | Ligand Name | No. | Species | (PDZ binding peptide) | NO |
| LIN-7 | NP_496982 | 91 | C. | 1 | Let-23 | NP_495962 | C. | EEAEAVQYENEEVSQKETCL | 184 |
| | | | elegans | | | | elegans | | |
| Dsh | NP_511118 | 92 | Droso- | 1 | Vang | NP_477177 | Droso- | EVVDPKSNKFVLKLNSETSV | 185 |
| | | | phila | | | | phila | | |
| Scribble | NP_733156 | 93 | Droso- | 2 | Gukh | NP_001097834 | Droso- | NKYVAAPVANPPLPSFETAL | 186 |
| | | | phila | | | | phila | | |
| Dlg1 | NP_727519 | 94 | Droso- | 1 | Fasciclin-II | NP_525066 | Droso- | FDGRFVHSRSGEIIGKNSAV | 187 |
| | | | phila | | | | phila | | |
| Dlg1 | NP_727519 | 94 | Droso- | 2 | Fasciclin-II | NP_525066 | Droso- | FDGRFVHSRSGEIIGKNSAV | 187 |
| | | | phila | | | | phila | | |
| Dlg1 | NP_727519 | 94 | Droso- | 1 | Shaker | NP_728123 | Droso- | SSGLTMRHNNALAVSIETDV | 188 |
| | | | phila | | | | phila | | |
| Dlg1 | NP_727519 | 94 | Droso- | 2 | Shaker | NP_728123 | Droso- | SSGLTMRHNNALAVSIETDV | 188 |
| | | | phila | | | | phila | | |
| Veli-2 | NP_071448 | 95 | human | 1 | BGT-1 | NP_003035 | human | NFGPSPTREGLIAGEKETHL | 189 |
| AF-6 | NP_001193937 | 96 | human | 1 | PVRL3 | NP_056295 | human | EDDLVSHVDGSVISRREWYV | 190 |
| AF-6 | NP_001193937 | 96 | human | 1 | EPHA7 | NP_004431 | human | SSIQTMRAQMLHLHGTGIQV | 191 |
| AF-6 | NP_001193937 | 96 | human | 1 | EPHB2 | NP_004433 | human | ILNSIQVMRAQMNQIQSVEV | 192 |
| AF-6 | NP_001193937 | 96 | human | 1 | PVRL1 | NP_002846 | human | AENMVSQNDGSFISKKEWYV | 193 |
| AF-6 | NP_001193937 | 96 | human | 1 | PVRL2 | NP_001036189 | human | SSPSDSYQGKGFVMSRAMYV | 194 |
| AF-6 | NP_001193937 | 96 | human | 1 | EPHB3 | NP_004434 | human | ILSSIQDMRLQMNQTLPVQV | 195 |
| AF-6 | NP_001193937 | 96 | human | 1 | EPHB6 | NP_058642 | human | YSQPSARSEGEFKQTSSFLV | 196 |
| AIE- | NP_005700 | 97 | human | 1 | Sans | NP_775748 | human | AVRRRRQAMERPPALEDTEL | 197 |
| 75/harmonin | | | | | | | | | |
| AIE- | NP_005700 | 97 | human | 1 | Usher syndrome | NP_114147 | human | RALGKPRPPLPPPQLGDTFL | 198 |
| 75/harmonin | | | | | type-1C protein- | | | | |
| | | | | | binding protein 1 | | | | |
| MAGI-2 | NP_036433 | 98 | human | 5 | Delta-catenin | NP_001323 | human | PYSELNYETSHYPASPDSWV | 199 |
| MAGI-2 | NP_036433 | 98 | human | 5 | GluR-delta2 | NP_001501 | human | QPTPTLGLNLGNDPDRGTSI | 200 |
| MAGI-2 | NP_036433 | 98 | human | 1 | Beta1 AR | NP_000675 | human | DSDSSLDEPCRPGFASESKV | 201 |
| MAGI-2 | NP_036433 | 98 | human | 1 | Pten | NP_000305 | human | DSDPENEPFDEDQHTQITKV | 202 |
| MAGI-2 | NP_036433 | 98 | human | 2 | RAPGEF2 | NP_055062 | human | PYQSQGFSTEEDEDEQVSAV | 203 |
| CASK | NP_001119526 | 99 | human | 1 | JAM | NP_058642 | human | YSQPSARSEGEFKQTSSFLV | 196 |
| CASK | NP_001119526 | 99 | human | 1 | Caspr2 | NP_054860 | human | MNNDPNFTETIDESKKEWLI | 204 |
| CASK | NP_001119526 | 99 | human | 1 | Caspr4 | NP_207837 | human | LKSELNIQNAVNENQKEYFF | 205 |
| CASK | NP_001119526 | 99 | human | 1 | Syndecan-1 | NP_002988 | human | PKQANGGAYQKPTKQEEFYA | 206 |
| CASK | NP_001119526 | 99 | human | 1 | Syndecan-2 | NP_002989 | human | GERKPSSAAYQKAPTKEFYA | 207 |
| CASK | NP_001119526 | 99 | human | 1 | Syndecan-3 | NP_055469 | human | EPKQASVTYQKPDKQEEFYA | 208 |
| CASK | NP_001119526 | 99 | human | 1 | Syndecan-4 | NP_002990 | human | SYDLGKKPIYKKAPTNEFYA | 209 |
| Delphilin | NP_001138590 | 100 | human | 1 | GluR-delta2 | NP_001501 | human | QPTPTLGLNLGNDPDRGTSI | 200 |
| InaD-like | NP_795352 | 101 | human | 8 | Claudin-1 | NP_066924 | human | SYPTPRPYPKPAPSSGKDYV | 201 |
| protein | | | | | | | | | |
| InaD-like | NP_795352 | 101 | human | 6 | ZO-3 | NP_055243 | human | VHDAESSDEDGYDWGPATDL | 202 |
| protein | | | | | | | | | |
| ERBIN | NP_061165 | 102 | human | 1 | ARVCF | NP_001661 | human | AVRLVDAVGDAKPQPVDSWV | 203 |
| ERBIN | NP_061165 | 102 | human | 1 | delta-catenin | NP_001323 | human | PYSELNYETSHYPASPDSWV | 199 |
| ERBIN | NP_061165 | 102 | human | 1 | PKP4 | NP_001005476 | human | STKRPSYRAEQYPGSPDSWV | 204 |
| GIPC | NP_974223 | 103 | human | 1 | 5T4 | NP_006661 | human | YRYEINADPRLTNLSSNSDV | 205 |
| GIPC | NP_974223 | 103 | human | 1 | Alpha-actinin-1 | NP_001123476 | human | VPGALDYMSFSTALYGESDL | 206 |
| GIPC | NP_974223 | 103 | human | 1 | Beta1-AR | NP_000675 | human | DSDSSLDEPCRPGFASESKV | 201 |
| GIPC | NP_974223 | 103 | human | 1 | TRP-1 | NP_000541 | human | QYQCYAEEYEKLQNPNQSVV | 207 |
| GIPC | NP_974223 | 103 | human | 1 | Integrin-alpha5 | NP_002196 | human | LPYGTAMEKAQLKPPATSDA | 208 |
| GIPC | NP_974223 | 103 | human | 1 | Integrin-alpha6A | NP_001073286 | human | IDNLEKKQWITKWNRNESYS | 209 |
| GOPC/PIST | NP_065132 | 104 | human | 1 | CFTR | NP_776297 | human | TDEEREETEEEVYLLNSTTL | 210 |
| IKEPP | NP_001161940 | 105 | human | 3 | GCC | NP_004954 | human | KKGTLEYLQLNTTDKESTYF | 211 |
| PTPL1 | NP_542414 | 106 | human | 4 | PARG1 | NP_004806 | human | PRLKRMQQFEDLEDEIPQFV | 212 |
| PTPL1 | NP542414 | 106 | human | 3 | PRK2 | NP_006247 | human | ILSEEEQEMFRDFDYIADWC | 213 |
| PTPL1 | NP_542414 | 106 | human | 2 | TRIP6 | NP_003293 | human | CKACSAWRIQELSATVTTDC | 214 |
| PTPL1 | NP_542414 | 106 | human | 5 | Ephrin-B1 | NP_004420 | human | PVYIVQEMPPQSPANIYYKV | 215 |
| PTPL1 | NP_542414 | 106 | human | 2 | FASLR | NP_690610 | human | KDITSDSENSNFRNEIQSLV | 216 |
| PTPL1 | NP542414 | 106 | human | 4 | FASLR | NP690610 | human | KDITSDSENSNFRNEIQSLV | 216 |
| PTPL1 | NP_542414 | 106 | human | 3 | p75 | NP_002498 | human | IQRADLVESLCSESTATSPV | 217 |
| PTPase- | NP_002821 | 107 | human | 1 | GluR-delta2 | NP_001501 | human | QPTPTLGLNLGNDPDRGTSI | 200 |
| MEG1 | | | | | | | | | |
| PTPase- | NP_002821 | 107 | human | 1 | NMDAR2A | NP_000824 | human | LNSCSNRRVYKKMPSIESDV | 218 |
| MEG1 | | | | | | | | | |
| MP55 | NP_071919 | 108 | human | 1 | CRB1 | NP_957705 | human | EGSRVEMWNLMPPPAMERLI | 219 |
| Shank1 | NP_057232 | 109 | human | 1 | DAP-1 | NP_004737 | human | SATESAESIEIYIPEAQTRL | 220 |
| Shank1 | NP_057232 | 109 | human | 1 | DAP12 | NP_004736 | human | SASERADSIEIYIPEAQTRL | 221 |
| Shank2 | NP_036441 | 110 | human | 1 | DAP-1 | NP_004737 | human | SATESAESIETYIPEAQTRL | 220 |
| Shank2 | NP_036441 | 110 | human | 1 | DAP-2 | NP_004736 | human | SASERADSIEIYIPEAQTRL | 221 |
| TIP-1 | NP_004243 | 111 | human | 1 | L-glutaminase | NP_037399 | human | ETQAEAAAEALSKENLESMV | 205 |
| TIP-1 | NP_004243 | 111 | human | 1 | Rhotekin | NP_149035 | human | PRTRGFCSKGQLRTWLQSPV | 206 |
| Veli-1 | NP_004655 | 112 | human | 1 | BGT-1 | NP_003035 | human | NFGPSPTREGLIAGEKETHL | 189 |
| Veli-2 | NP_071448 | 95 | human | 1 | Kir2.3 | NP_004972 | human | ERMQASLPLDNISYRRESAI | 222 |
| Veli-3 | NP_060832 | 113 | human | 1 | NR2B | NP_000825 | human | FNGSSNGHVYEKLSSIESDV | 223 |
| Veli-3 | NP_060832 | 113 | human | 1 | BGT-1 | NP_003035 | human | NFGPSPTREGLIAGEKETHL | 189 |
| ZO-1 | NP_003248 | 114 | human | 2 | Connexin-43 | NP_000156 | human | PSSRASSRASSRPRPDDLEI | 224 |
| ZO-1 | NP_003248 | 114 | human | 2 | Connexin-45 | NP_005488 | human | GSNKSTASSKSGDGKNSVWI | 115 |
| SAP102 | NP_005488 | 115 | human | 1 | PMCA4b | NP_001675 | human | CNQVQLPQSDSSLQSLETSV | 210 |
| SAP102 | NP_005488 | 115 | human | 2 | PMCA4b | NP_001675 | human | CNQVQLPQSDSSLQSLETSV | 210 |
| SAP102 | NP_005488 | 115 | human | 1 | p51-nedasin | NP_004284 | human | RNIEEVYVGGKQVVPFSSSV | 225 |
| SAP97 | NP_001191316 | 116 | human | 1 | PMCA2b | NP_001674 | human | SKSATSSSPGSPIHSLETSL | 216 |
| SAP97 | NP_001191316 | 116 | human | 2 | PMCA2b | NP_001674 | human | SKSATSSSPGSPIHSLETSL | 216 |
| SAP97 | NP_001191316 | 116 | human | 1 | PMCA4b | NP_001675 | human | CNQVQLPQSDSSLQSLETSV | 210 |
| SAP97 | NP_001191316 | 116 | human | 2 | PMCA4b | NP_001675 | human | CNQVQLPQSDSSLQSLETSV | 210 |
| SAP97 | NP_001191316 | 116 | human | 2 | PBK | NP_060962 | human | EDPKDRPSAAHIVEALETDV | 226 |
| MUPP1 | NP_003820 | 117 | human | 10 | 5HT-2C | NP_000859 | human | ENLELPVNPSSVVSERISSV | 227 |
| MUPP1 | NP_003820 | 117 | human | 10 | TAPP-1 | NP_067635 | human | QEKDCDLVDLDDASLPVSDV | 228 |
| MUPP1 | NP_003820 | 117 | human | 13 | TAPP-1 | NP_067635 | human | QEKDCDLVDLDDASLPVSDV | 228 |
| MUPP1 | NP_003820 | 117 | human | 10 | TAPP-2 | NP_067636 | human | LKEKAFPFDLDDDSIRTSDV | 229 |
| MUPP1 | NP_003820 | 117 | human | 13 | TAPP-2 | NP_067636 | human | LKEKAFPFDLDDDSIRTSDV | 229 |
| MUPP1 | NP_003820 | 117 | human | 10 | KIT | NP_000213 | human | INSVGSTASSSQPLLVHDDV | 230 |
| NHERF-1 | NP_004243 | 111 | human | 1 | CFTR | NP_000483 | human | KPQIAALKEETEEEVQDTRL | 231 |
| NHERF-1 | NP_004243 | 111 | human | 1 | EPI64 | NP_001191169 | human | SAHHRSQESLTSQESEDTYL | 232 |
| NHERF-1 | NP_004243 | 111 | human | 2 | GRK6 | NP_001004106 | human | SRQDCCGNCSDSEEELPTRL | 233 |
| NHERF-1 | NP_004243 | 111 | human | 1 | P2Y1 | NP_002554 | human | SEDMTLNILPEFKQNGDTSL | 234 |
| NHERF-1 | NP_004243 | 111 | human | 1 | PAG | NP_060910 | human | KENDYESISDLQQGRDITRL | 235 |
| NHERF-1 | NP_004243 | 111 | human | 2 | YAP65 | NP_001123617 | human | DMESVLAATKLDKESFLTWL | 236 |
| NHERF-1 | NP_004243 | 111 | human | 1 | Beta2-AR | NP_000015 | human | VPSDNIDSQGRNCSTNDSLL | 237 |
| NHERF-1 | NP_004243 | 111 | human | 1 | DRA | NP_000102 | human | INTNGGLRNRVYEVPVETKF | 238 |
| NHERF-1 | NP_004243 | 111 | human | 1 | KOR-1 | NP_000903 | human | RNTVQDPAYLRDIDGMNKPV | 239 |
| NHERF-2 | NP_004243 | 111 | human | 2 | Clcn3c | NP_776297 | human | TDEEREETEEEVYLLNSTTL | 210 |
| NHERF-2 | NP_004243 | 111 | human | 1 | EPI64 | NP_001191169 | human | SAHHRSQESLTSQESEDTYL | 232 |
| NHERF-2 | NP_004243 | 111 | human | 2 | PLC-beta3 | NP_001171812 | human | GSSGHLSGADSESQEENTQL | 240 |
| NHERF-2 | NP_004243 | 111 | human | 1 | Sgk1 | NP_005618 | human | KEAAEAFLGFSYAPPTDSFL | 241 |
| NHERF-2 | NP_004243 | 111 | human | 1 | beta2-AR | NP_000015 | human | VPSDNIDSQGRNCSTNDSLL | 237 |
| NHERF-2 | NP_004243 | 111 | human | 1 | SRY | NP_003131 | human | PINAASSPQQRDRYSHWTKL | 242 |
| NHERF-2 | NP_004243 | 111 | human | 2 | SRY | NP_003131 | human | PINAASSPQQRDRYSHWTKL | 242 |
| PDZ- | NP_056128 | 118 | human | 1 | IGF-1R | NP_000866 | human | HMNGGRKNERALPLPQSSTC | 243 |
| RhoGEF | | | | | | | | | |
| PDZ- | NP_056128 | 118 | human | 1 | PlexinB1 | NP_002664 | human | QLGYRLQQIAAAVENKVTDL | 244 |
| RhoGEF | | | | | | | | | |
| PDZ- | NP_056128 | 118 | human | 1 | PlexinB2 | NP_036533 | human | QLAFRLQQIAAALENKVTDL | 245 |
| RhoGEF | | | | | | | | | |
| PDZ- | NP_056128 | 118 | human | 1 | PlexinB3 | NP_001156729 | human | QLACRLQQVAALVENKVTDL | 246 |
| RhoGEF | | | | | | | | | |
| PDZK1 | NP_001188255 | 119 | human | 1 | Clcn3c | NP_776297 | human | TDEEREETEEEVYLLNSTTL | 210 |
| PICK1 | NP_036539 | 120 | human | 1 | ARF1 | NP_001649 | human | SGDGLYEGLDWLSNQLRNQK | 247 |
| PICK1 | NP_036539 | 120 | human | 1 | ARF3 | NP_001650 | human | SGDGLYEGLDWLANQLKNKK | 248 |
| PICK1 | NP_036539 | 120 | human | 1 | DAT | NP_001035 | human | RELVDRGEVRQFTLRHWLKV | 249 |
| PICK1 | NP_036539 | 120 | human | 1 | ERBB2 | NP_004439 | human | TFKGTPTAENPEYLGLDVPV | 250 |
| PICK1 | NP_036539 | 120 | human | 1 | NET | NP_001034 | human | HHLVAQRDIRQFQLQHWLAI | 251 |
| PSD-93 | NP_001193698 | 121 | human | 1 | PMCA2b | NP_001674 | human | SKSATSSSPGSPIHSLETSL | 216 |
| PSD-93 | NP_001193698 | 121 | human | 2 | PMCA2b | NP_001674 | human | SKSATSSSPGSPIHSLETSL | 216 |
| PSD-93 | NP_001193698 | 121 | human | 1 | PMCA4b | NP_001675 | human | CNQVQLPQSDSSLQSLETSV | 210 |
| PSD-93 | NP_001193698 | 121 | human | 2 | PMCA4b | NP_001675 | human | CNQVQLPQSDSSLQSLETSV | 210 |
| PSD-95 | NP_001356 | 122 | human | 1 | PMCA2b | NP_001674 | human | SKSATSSSPGSPIHSLETSL | 216 |
| PSD-95 | NP_001356 | 122 | human | 2 | PMCA2b | NP_001674 | human | SKSATSSSPGSPIHSLETSL | 216 |
| PSD-95 | NP_001356 | 122 | human | 1 | PMCA4b | NP_001675 | human | CNQVQLPQSDSSLQSLETSV | 210 |
| PSD-95 | NP_001356 | 122 | human | 2 | PMCA4b | NP_001675 | human | CNQVQLPQSDSSLQSLETSV | 210 |
| alpha-1- | NP_003089 | 123 | human | 1 | SAPK3 | NP_000325 | human | WPPAPPPGQTVRPGVKESLV | 252 |
| syntrophin | | | | | | | | | |
| alpha-1- | NP_003089 | 123 | human | 1 | SKM1 | NP_001153633 | human | HSEDLADFPPSPDRDRESIV | 253 |
| syntrophin | | | | | | | | | |
| alpha-1- | NP_003089 | 123 | human | 1 | L-glutaminase | NP_037399 | human | ETQAEAAAEALSKENLESMV | 205 |
| syntrophin | | | | | | | | | |
| beta-2- | NP_006741 | 124 | human | 1 | ABCA1 | NP_005493 | human | VDVAVLTSFLQDEKVKESYV | 254 |
| syntrophin | | | | | | | | | |
| beta-2- | NP_006741 | 124 | human | 1 | ERBB4 | NP_001036064 | human | SLKPGTVLPPPPYRHRNTVV | 255 |
| syntrophin | | | | | | | | | |
| gamma-1- | NP_061840 | 125 | human | 1 | DGKZ | NP_001099010 | human | YLENRQHYQMIQREDQETAV | 256 |
| syntrophin | | | | | | | | | |
| gamma-2- | NP_061841 | 126 | human | 1 | SKM2 | NP_001153633 | human | HSEDLADFPPSPDRDRESIV | 253 |
| syntrophin | | | | | | | | | |
| p55 | NP_001159933 | 127 | human | 1 | Glycophorin-C | NP_058131 | human | LQGDPALQDAGDSSRKEYFI | 257 |
| PTP-H1 | NP_001138843 | 128 | human | 1 | ADAM17 | NP_003174 | human | KAASFKLQRQNRVDSKETEC | 258 |
| MAGI-2 | NP_036433 | 98 | human | 1 | HPV-16-E6 | NP_040310 | Papillo- | HSCCNRARQERLQRRRETQV | 259 |
| | | | | | | | mavirus | | |
| MAGI-3 | NP_001136254 | 129 | human | 1 | HPV-16-E6 | NP_040310 | Papillo- | HSCCNRARQERLQRRRETQV | 259 |
| | | | | | | | mavirus | | |
| alpha-1- | NP_003089 | 123 | mouse | 1 | aquaporin-4 | NP_001641 | mouse | IDRGEEKKGRDSSGEVLSSV | 260 |
| syntrophin | | | | | | | | | |
| ASIP/PAR3 | NP_001013599 | 130 | mouse | 1 | PVRL1 | NP_067399 | mouse | AENMVSQNDGSFISKKEWYV | 261 |
| ASIP/PAR3 | NP_001013599 | 130 | mouse | 1 | PVRL3 | NP_067470 | mouse | EDGLVSHVDGSVISRREWYV | 262 |
| ASIP/PAR3 | NP_001013599 | 130 | mouse | 1 | JAM | NP_766235 | mouse | YSQPSARSEGEFKQTSSFLV | 263 |
| Cipp | NP_031730 | 131 | mouse | 4 | DRASIC | NP_892045 | mouse | CAVTKTLSASHRTCYLVTRL | 264 |
| Cipp | NP_031730 | 131 | mouse | 2 | NR2B | NP_032197 | mouse | FNGSSNGHVYEKLSSIESDV | 265 |
| Cipp | NP_031730 | 131 | mouse | 3 | NR2B | NP_032197 | mouse | FNGSSNGHVYEKLSSIESDV | 265 |
| Cipp | NP_031730 | 131 | mouse | 3 | NR2C | NP_034480 | mouse | TQGFPRSCTWRRISSLESEV | 266 |
| Cipp | NP_031730 | 131 | mouse | 2 | Kir4.1 | NP_001034573 | mouse | SLREQAEKEGSALSVRISNV | 267 |
| Cipp | NP_031730 | 131 | mouse | 2 | Kir4.2 | NP_062638 | mouse | RQEDQRERELRSLLLQQSNV | 268 |
| Cipp | NP_031730 | 131 | mouse | 3 | kir4.2 | NP_062638 | mouse | RQEDQRERELRSLLLQQSNV | 268 |
| Cypher1c | NP_001034164 | 132 | mouse | 1 | alpha-actinin-2 | NP_150371 | mouse | VPGALDYTAFSSALYGESDL | 269 |
| DVL-1 | NP_034221 | 133 | mouse | 1 | Vang | NP_277044 | mouse | EFVDPKSHKFVMRLQSETSV | 270 |
| Enigma | NP_001177782 | 134 | mouse | 1 | beta-tropomyosin | NP_033442 | mouse | MKYKAISEELDNALNDITSL | 271 |
| ERBIN | NP_001005868 | 135 | mouse | 1 | ERBB2 | NP_001003817 | mouse | TFEGTPTAENPEYLGLDVPV | 272 |
| GIPC | NP_061241 | 136 | mouse | 1 | Glut1 | NP_035530 | mouse | SQSDKTPEELFHPLGADSQV | 273 |
| GIPC | NP_061241 | 136 | mouse | 1 | KIF1B | NP_032467 | mouse | PRMRRQFSAPNLKAGRETTV | 274 |
| GIPC | NP_061241 | 136 | mouse | 1 | Semaphorin-4C | NP_001119519 | mouse | RRKLQQRQPLPDSNPEESSV | 275 |
| GIPC | NP_061241 | 136 | mouse | 1 | TGFR-3 | NP_035708 | mouse | SAAHSIGSTQSTPCSSSSTA | 276 |
| GOPC/PIST | NP_001186201 | 137 | mouse | 1 | Frizzled-1 | NP_067432 | mouse | NSWRKFYTRLTNSKQGETTV | 277 |
| MAGI-1 | NP_034497 | 138 | mouse | 5 | beta-catenin | NP_031640 | mouse | LMDGLPPGDSNQLAWFDTDL | 278 |
| MAGI-1 | NP_034497 | 138 | mouse | 1 | NET1 | NP_001040624 | mouse | GLRRARDKAQSGGKKKETLV | 279 |
| Veli-1 | NP_001028395 | 139 | mouse | 1 | NR2B | NP_032197 | mouse | FNGSSNGHVYEKLSSIESDV | 265 |
| Veli-2 | NP_035828 | 140 | mouse | 1 | NR2B | NP_032197 | mouse | FNGSSNGHVYEKLSSIESDV | 265 |
| ZO-1 | NP_033412 | 141 | mouse | 1 | Claudin-1 | NP_057883 | mouse | SYPTPRPYPKPTPSSGKDYV | 280 |
| ZO-2 | NP_001185914 | 142 | mouse | 1 | Claudin-1 | NP_057883 | mouse | SYPTPRPYPKPTPSSGKDYV | 280 |
| SAP102 | NP_058027 | 143 | mouse | 1 | BAI1 | NP_778156 | mouse | KAGATIPLVGQDIIDLQTEV | 281 |
| SAP102 | NP_058027 | 143 | mouse | 2 | BAI1 | NP_778156 | mouse | KAGATIPLVGQDIIDLQTEV | 281 |
| MUPP1 | NP_034950 | 144 | mouse | 9 | JAM | NP_766235 | mouse | YSQPSTRSEGEFKQTSSFLV | 263 |
| MUPP1 | NP_034950 | 144 | mouse | 10 | Claudin-1 | NP_057883 | mouse | SYPTPRPYPKPTPSSGKDYV | 280 |
| NHERF-1 | NP_036160 | 145 | mouse | 1 | PLC-beta1 | NP_001139302 | mouse | PPSSEELEGENPGKEFDTPL | 282 |
| NHERF-1 | NP_036160 | 145 | mouse | 1 | TrpC4 | NP_058680 | mouse | DYDLSPTDTAAHEDYVTTRL | 283 |
| NHERF-1 | NP_036160 | 145 | mouse | 1 | TrpC5 | NP_033454 | mouse | CDLLMHKWGDGQEEQVTTRL | 284 |
| NHERF-1 | NP_036160 | 145 | mouse | 1 | NaPi-7 | NP_035522 | mouse | PPATPSPRLALPAHHNATRL | 285 |
| NHERF-2 | NP_075542 | 146 | mouse | 1 | Taz | NP_598545 | mouse | PLFNDVESVLNKSEPELTWL | 286 |
| NHERF-2 | NP_075542 | 146 | mouse | 1 | NaPi-7 | NP_035522 | mouse | PPATPSPRLALPAHHNATRL | 285 |
| IL-16 | NP_034681 | 147 | mouse | 1 | MBC | NP_001153006 | mouse | LGRGRSEEALADSRSYVSNL | 287 |
| PDZ-RGS3 | NP_599018 | 148 | mouse | 1 | Ephrin-B1 | NP_034240 | mouse | PVYIVQEMPPQSPANIYYKV | 288 |
| PDZK1 | NP_067492 | 149 | mouse | 1 | CFTR | NP_066388 | mouse | RTQITALKEETEEEVQETRL | 289 |
| PDZK1 | NP_067492 | 149 | mouse | 3 | CFTR | NP_066388 | mouse | RTQITALKEETEEEVQETRL | 289 |
| PDZK1 | NP_067492 | 149 | mouse | 4 | CFTR | NP_066388 | mouse | RTQITALKEETEEEVQETRL | 289 |
| PDZK1 | NP_067492 | 149 | mouse | 3 | NaPi-7 | NP_035522 | mouse | PPATPSPRLALPAHHNATRL | 285 |
| PICK1 | NP_032863 | 150 | mouse | 1 | PKC-A | NP_035231 | mouse | FEGFSYVNPQFVHPILQSAV | 290 |
| PSD-93 | NP_035937 | 151 | mouse | 1 | Frizzled-1 | NP_067432 | mouse | NSWRKFYTRLTNSKQGETTV | 277 |
| PSD-93 | NP_035937 | 151 | mouse | 2 | Frizzled-1 | NP_067432 | mouse | NSWRKFYTRLTNSKQGETTV | 277 |
| PSD-93 | NP_035937 | 151 | mouse | 1 | Frizzled-4 | NP_032081 | mouse | KREKRGNGWVKPGKGNETVV | 291 |
| PSD-93 | NP_035937 | 151 | mouse | 2 | Frizzled-4 | NP_032081 | mouse | KREKRGNGWVKPGKGNETVV | 291 |
| PSD-93 | NP_035937 | 151 | mouse | 1 | Frizzled-7 | NP_032083 | mouse | QSWRRFYHRLSHSSKGETAV | 292 |
| PSD-95 | NP_001103222 | 152 | mouse | 1 | Frizzled-1 | NP_067432 | mouse | NSWRKFYTRLTNSKQGETTV | 277 |
| PSD-95 | NP_001103222 | 152 | mouse | 2 | Frizzled-1 | NP_067432 | mouse | NSWRKFYTRLTNSKQGETTV | 277 |
| PSD-95 | NP_001103222 | 152 | mouse | 1 | Frizzled-2 | NP_065256 | mouse | HSWRKFYTRLTNSRHGETTV | 293 |
| PSD-95 | NP_001103222 | 152 | mouse | 2 | Frizzled-2 | NP_065256 | mouse | HSWRKFYTRLTNSRHGETTV | 293 |
| PSD-95 | NP_001103222 | 152 | mouse | 1 | Frizzled-4 | NP_032081 | mouse | KREKRGNGWVKPGKGNETVV | 291 |
| PSD-95 | NP_001103222 | 152 | mouse | 2 | Frizzled-4 | NP_032081 | mouse | KREKRGNGWVKPGKGNETVV | 291 |
| ZO-3 | NP_038797 | 153 | mouse | 1 | Claudin-1 | NP_057883 | mouse | SYPTPRPYPKPTPSSGKDYV | 280 |
| Inx1 | NP_001153049 | 154 | mouse | 2 | CAR-1 | NP_034118 | mouse | PTLAPAKFKYAYKTDGITVV | 294 |
| Inx1 | NP_001153049 | 154 | mouse | 2 | CAR-2 | NP_001020363 | mouse | SRMGAVPVMIPAQSKDGSIV | 295 |
| nNos | NP_032738 | 155 | mouse | 1 | CtBP1 | NP_001185790 | mouse | PSPGQTVKPEADRDHTSDQL | 296 |
| Rhophilin1 | NP_001156937 | 156 | mouse | 1 | Ropporin | NP_109669 | mouse | GPDGLIKVNDFTQNPRVRLE | 297 |
| NHERF-2 | NP_001075576 | 157 | rabbit | 1 | Podocalyxin | NP_001076235 | rabbit | IVPLDNLTKDDLDEEEDTHL | 298 |
| MAGI-2 | NP_036433 | 98 | rat | 1 | neuroligin-1 | NP_446320 | rat | QNNTLPHPHPHPHSHSTTRV | 299 |
| Glutamate | NP_612544 | 158 | rat | 3 | glutamate | NP_001077280 | rat | QNFATYKEGYNVYGIESVKI | 300 |
| receptor- | | | | | receptor 2 | | | | |
| interacting | | | | | | | | | |
| protein 2 | | | | | | | | | |
| Glutamate | NP_612544 | 158 | rat | 5 | glutamate | NP_116785 | rat | QNYATYREGYNVYGTESVKI | 301 |
| receptor- | | | | | receptor 3 | | | | |
| interacting | | | | | | | | | |
| protein 2 | | | | | | | | | |
| MAGI-2 | NP_446073 | 159 | rat | 5 | NMDAR2C | NP_113759 | rat | IVTVVTMVTNVDFPPKESSL | 302 |
| CASK | NP_071520 | 160 | rat | 1 | Neurexin-1-alpha | NP_068535 | rat | PSSAKSANKNKKNKDKEYYV | 303 |
| CASK | NP_071520 | 160 | rat | 1 | Neurexin-beta | NP_068535 | rat | PSSAKSANKNKKNKDKEYYV | 303 |
| CASK | NP_071520 | 160 | rat | 1 | Parkin | NP_064478 | rat | CWNCGCEWNRACMGDHWFDV | 304 |
| Densin-180 | NP_476483 | 161 | rat | 1 | Maguin-1 | NP_001106837 | rat | EVDVITSSLTHTHSYIETHV | 305 |
| Densin-180 | NP_476483 | 161 | rat | 1 | delta-catenin | XP_001065606 | rat | PYSELNYETSHYPASPDSWV | 306 |
| GIPC | NP_445793 | 162 | rat | 1 | RGS-GAIP | NP_067693 | rat | LTSPTYRSLLLQGAPQSSEA | 307 |
| GIPC | NP_445793 | 162 | rat | 1 | Neuropilin-1 | NP_659566 | rat | VDGVKLKKDKLNPQSNYSEA | 308 |
| GIPC | NP_445793 | 162 | rat | 1 | Syndecan-1 | NP_037158 | rat | PKQANGGAYQKPTKQEEFYA | 309 |
| GRIP | NP_114458 | 163 | rat | 6 | Liprin-alpha-2 | NP_001102215 | rat | DVASSRLQRLDNSTVRTYSC | 310 |
| RGS12 | NP_062212 | 164 | rat | 1 | IL-8R | NP_058879 | rat | AKEGRPSFVGSSSANTSTTL | 311 |
| Shank1 | NP_113939 | 165 | rat | 1 | SS2R | NP_062221 | rat | LNETTETQRTLLNGDLQTSI | 312 |
| Shank1 | NP_113939 | 165 | rat | 1 | Beta-Pix | NP_001106994 | rat | KLVRKVLKNMNDPAWDETNL | 313 |
| Shank2 | NP_958738 | 166 | rat | 1 | CIRL-1 | NP_075251 | rat | PSLEGPGPDGDGQMQLVTSL | 314 |
| Shank2 | NP_958738 | 166 | rat | 1 | CIRL-2 | NP_599235 | rat | EGCIPEGDVREGQMQLVTSL | 315 |
| Shank3 | NP_067708 | 167 | rat | 1 | mGluR5 | NP_058708 | rat | SSPKYDTLIIRDYTQSSSSL | 316 |
| Shank3 | NP_067708 | 167 | rat | 1 | DAP-1 | NP_075235 | rat | SATESAESIEIYIPEAQTRL | 317 |
| Shank3 | NP_067708 | 167 | rat | 1 | DAP-2 | NP_446353 | rat | SATERADSIEIYIPEAQTRL | 318 |
| Shank3 | NP_067708 | 167 | rat | 1 | mGluR1a | NP_058707 | rat | PGGRQAPKGQHVWQRLSVHV | 319 |
| Tamalin | NP_620249 | 168 | rat | 1 | GABAB2 | NP_113990 | rat | TASPRHRHVPPSFRVMVSGL | 320 |
| Tamalin | NP_620249 | 168 | rat | 1 | DAP1 | NP_075235 | rat | SATESAESIEIYIPEAQTRL | 317 |
| Tamalin | NP_620249 | 168 | rat | 1 | DAP3 | NP_775168 | rat | SATESADSIEIYIPEAQTRL | 321 |
| Tamalin | NP_620249 | 168 | rat | 1 | mGluR1a | NP_058707 | rat | PNVTYASVILRDYKQSSSTL | 319 |
| Tamalin | NP_620249 | 168 | rat | 1 | mGluR5 | NP_058708 | rat | SSPKYDTLIIRDYTQSSSSL | 316 |
| RIM1 | NP_439894 | 169 | rat | 1 | ERC-2 | NP_740768 | rat | SQHSNHRPSPDQDDEEGIWA | 322 |
| RIM1 | NP_439894 | 169 | rat | 1 | ERC-1b | NP_740769 | rat | SNQTNHKPSPDQDEEEGIWA | 323 |
| SAP102 | NP_113827 | 170 | rat | 1 | ERBB4 | NP_067719 | rat | SLKPGTMLPPPPYRHRNTVV | 324 |
| SAP102 | NP_113827 | 170 | rat | 2 | ERBB4 | NP_067719 | rat | SLKPGTMLPPPPYRHRNTVV | 324 |
| SAP102 | NP_113827 | 170 | rat | 1 | NMDAR2A | NP_036705 | rat | LNSCSNRRVYKKMPSIESDV | 325 |
| SAP102 | NP_113827 | 170 | rat | 2 | NMDAR2A | NP_036705 | rat | LNSCSNRRVYKKMPSIESDV | 325 |
| SAP102 | NP_113827 | 170 | rat | 3 | NMDAR2A | NP_036705 | rat | LNSCSNRRVYKKMPSIESDV | 325 |
| SAP102 | NP_113827 | 170 | rat | 1 | NMDAR2B | NP_036706 | rat | FNGSSNGHVYEKLSSIESDV | 326 |
| SAP102 | NP_113827 | 170 | rat | 2 | NMDAR2B | NP_036706 | rat | FNGSSNGHVYEKLSSIESDV | 326 |
| SAP102 | NP_113827 | 170 | rat | 1 | PKC-A | NP_001099183 | rat | FEGFSYVNPQFVHPILQSAV | 327 |
| SAP102 | NP_113827 | 170 | rat | 2 | PKC-A | NP_001099183 | rat | FEGFSYVNPQFVHPILQSAV | 327 |
| SAP102 | NP_113827 | 170 | rat | 3 | SynGAP | NP_851606 | rat | KRLLDAQRGSFPPWVQQTKV | 328 |
| SAP97 | NP_036920 | 171 | rat | 1 | Frizzled-1 | NP_067089 | rat | NSWRKFYTRLTNSKQGETTV | 329 |
| SAP97 | NP_036920 | 171 | rat | 2 | Frizzled-1 | NP_067089 | rat | NSWRKFYTRLTNSKQGETTV | 329 |
| SAP97 | NP_036920 | 171 | rat | 1 | Frizzled-4 | NP_072145 | rat | KREKRGNGWVKPGKGNETVV | 330 |
| SAP97 | NP_036920 | 171 | rat | 2 | Frizzled-4 | NP_072145 | rat | KREKRGNGWVKPGKGNETVV | 330 |
| SAP97 | NP_036920 | 171 | rat | 1 | NMDAR2A | NP_036705 | rat | LNSCSNRRVYKKMPSIESDV | 325 |
| SAP97 | NP_036920 | 171 | rat | 1 | NMDAR2B | NP_036706 | rat | FNGSSNGHVYEKLSSIESDV | 326 |
| SAP97 | NP_036920 | 171 | rat | 3 | ADAM17 | NP_064702 | rat | KAASFKLQRQSRVDSKETEC | 331 |
| SAP97 | NP_036920 | 171 | rat | 2 | Kir2.2 | NP_446433 | rat | DRLQASSGALERPYRRESEI | 332 |
| MUPP1 | NP_062069 | 172 | rat | 1 | AN2/NG2 | NP_112284 | rat | ELLQFCRTPNPALRNGQYWV | 333 |
| Neurabin-1 | NP_445925 | 173 | rat | 1 | p70/S6K | NP_114191 | rat | YKKQAFPMISKRPEHLRMNL | 334 |
| Omp25 | NP_072121 | 174 | rat | 1 | Synaptojanin-2 | NP_072121 | rat | FVRTVAAQRLTPVDASGSSV | 174 |
| PICK1 | NP_445912 | 175 | rat | 1 | BNaC1 | NP_037024 | rat | SHTVNVPLQTALGTLEEIAC | 335 |
| PICK1 | NP_445912 | 175 | rat | 1 | BNaC2 | NP_077068 | rat | YAANILPHHPARGTFEDFTC | 336 |
| PICK1 | NP_445912 | 175 | rat | 1 | GluR2 | NP_058957 | rat | QNFATYKEGYNVYGIESVKI | 337 |
| PICK1 | NP_445912 | 175 | rat | 1 | GluR3 | NP_116785 | rat | QNYATYREGYNVYGTESVKI | 301 |
| PICK1 | NP_445912 | 175 | rat | 1 | GluR4 | NP_116785 | rat | HNLATYREGYNVYGTESIKI | 301 |
| PICK1 | NP_445912 | 175 | rat | 1 | PrRPR | NP_631932 | rat | LSWPRKIVPHGQNMTVSVVI | 338 |
| PICK1 | NP_445912 | 175 | rat | 1 | mGluR3 | NP_001099182 | rat | TYVPTVCNGREVLDSTTSSL | 339 |
| PICK1 | NP_445912 | 175 | rat | 1 | mGluR7a | NP_112302 | rat | VDPNSPAAKKKYVSYNNLVI | 340 |
| PSD-93 | NP_071618 | 176 | rat | 1 | ERBB4 | NP_067719 | rat | SLKPGTMLPPPPYRHRNTVV | 324 |
| PSD-93 | NP_071618 | 176 | rat | 2 | ERBB4 | NP_067719 | rat | SLKPGTMLPPPPYRHRNTVV | 324 |
| PSD-93 | NP_071618 | 176 | rat | 2 | GluR-delta2 | NP_077355 | rat | QPTPTLGLNLGNDPDRGTSI | 341 |
| PSD-93 | NP_071618 | 176 | rat | 1 | NMDAR2A | NP_036705 | rat | LNSCSNRRVYKKMPSIESDV | 325 |
| PSD-93 | NP_071618 | 176 | rat | 2 | NMDAR2A | NP_036705 | rat | LNSCSNRRVYKKMPSIESDV | 325 |
| PSD-93 | NP_071618 | 176 | rat | 1 | NMDAR2B | NP_036706 | rat | FNGSSNGHVYEKLSSIESDV | 326 |
| PSD-93 | NP_071618 | 176 | rat | 2 | NMDAR2B | NP_036706 | rat | FNGSSNGHVYEKLSSIESDV | 326 |
| PSD-93 | NP_071618 | 176 | rat | 1 | Kv1.4 | NP_037103 | rat | DDSETDKNNCSNAKAVETDV | 342 |
| PSD-93 | NP_071618 | 176 | rat | 2 | Kv1.4 | NP_037103 | rat | DDSETDKNNCSNAKAVETDV | 342 |
| PSD-95 | NP_062567 | 177 | rat | 1 | CRIPT | NP_063972 | rat | ICAMCGKKVLDTKNYKQTSV | 343 |
| PSD-95 | NP_062567 | 177 | rat | 1 | ERBB4 | NP_067719 | rat | SLKPGTMLPPPPYRHRNTVV | 324 |
| PSD-95 | NP_062567 | 177 | rat | 2 | ERBB4 | NP_067719 | rat | SLKPGTMLPPPPYRHRNTVV | 324 |
| PSD-95 | NP_062567 | 177 | rat | 1 | GluR6 | NP_062182 | rat | EVINMHTFNDRRLPGKETMA | 344 |
| PSD-95 | NP_062567 | 177 | rat | 1 | NMDAR2A | NP_036705 | rat | LNSCSNRRVYKKMPSIESDV | 325 |
| PSD-95 | NP_062567 | 177 | rat | 2 | NMDAR2A | NP_036705 | rat | LNSCSNRRVYKKMPSIESDV | 325 |
| PSD-95 | NP_062567 | 177 | rat | 1 | NMDAR2B | NP_036706 | rat | FNGSSNGHVYEKLSSIESDV | 326 |
| PSD-95 | NP_062567 | 177 | rat | 2 | NMDAR2B | NP_036706 | rat | FNGSSNGHVYEKLSSIESDV | 326 |
| PSD-95 | NP_062567 | 177 | rat | 1 | PKC-A | NP_001099183 | rat | FEGFSYVNPQFVHPILQSAV | 327 |
| PSD-95 | NP_062567 | 177 | rat | 2 | PKC-A | NP_001099183 | rat | FEGFSYVNPQFVHPILQSAV | 327 |
| PSD-95 | NP_062567 | 177 | rat | 3 | GCS-alpha-2 | NP_076446 | rat | SRIKKVSYNIGTMFLRETSL | 345 |
| PSD-95 | NP_062567 | 177 | rat | 1 | Kv1.4 | NP_037103 | rat | DDSETDKNNCSNAKAVETDV | 342 |
| PSD-95 | NP_062567 | 177 | rat | 2 | Kv1.4 | NP_037103 | rat | DDSETDKNNCSNAKAVETDV | 342 |
| PSD-95 | NP_062567 | 177 | rat | 1 | Sec8 | NP_446327 | rat | IICEQAAIKQATKDKKITTV | 346 |
| PSD-95 | NP_062567 | 177 | rat | 1 | SynGAP | NP_851606 | rat | KRLLDAQRGSFPPWVQQTRV | 328 |
| PSD-95 | NP_062567 | 177 | rat | 2 | SynGAP | NP_851606 | rat | KRLLDAQRGSFPPWVQQTRV | 328 |
| PSD-95 | NP_062567 | 177 | rat | 3 | SynGAP | NP_851606 | rat | KRLLDAQRGSFPPWVQQTRV | 328 |
| Mint1 | NP_113967 | 178 | rat | 1 | NaPi-7 | NP_001182128 | rat | LSTGVRARHSYHHPDQDHWC | 347 |
| Mint1 | NP_113967 | 178 | rat | 1 | Presenilin-1 | NP_062036 | rat | ATDYLVQPFMDQLAFHQFYI | 348 |
| Mint1 | NP_113967 | 178 | rat | 2 | Presenilin-1 | NP_062036 | rat | ATDYLVQPFMDQLAFHQFYI | 348 |
| Mint1 | NP_113967 | 178 | rat | 1 | Presenilin-2 | NP_112349 | rat | STDNLVRPFMDTLASHQLYI | 349 |
| Mint1 | NP_113967 | 178 | rat | 2 | Presenilin-2 | NP_112349 | rat | STDNLVRPFMDTLASHQLYI | 349 |
| Mint2 | NP_113968 | 179 | rat | 1 | Presenilin-1 | NP_062036 | rat | ATDYLVQPFMDQLAFHQFYI | 348 |
| Mint2 | NP_113968 | 179 | rat | 2 | Presenilin-1 | NP_062036 | rat | ATDYLVQPFMDQLAFHQFYI | 348 |
| Mint2 | NP_113968 | 179 | rat | 1 | Presenilin-2 | NP_112349 | rat | STDNLVRPFMDTLASHQLYI | 349 |
| Mint2 | NP_113968 | 179 | rat | 2 | Presenilin-2 | NP_112349 | rat | STDNLVRPFMDTLASHQLYI | 349 |
| Mint3 | NP_113969 | 180 | rat | 1 | Presenilin-1 | NP_062036 | rat | ATDYLVQPFMDQLAFHQFYI | 348 |
| Mint3 | NP_113969 | 180 | rat | 2 | Presenilin-1 | NP_062036 | rat | ATDYLVQPFMDQLAFHQFYI | 348 |
| Mint3 | NP_113969 | 180 | rat | 1 | Presenilin-2 | NP_112349 | rat | STDNLVRPFMDTLASHQLYI | 349 |
| Mint3 | NP_113969 | 180 | rat | 2 | Presenilin-2 | NP_112349 | rat | STDNLVRPFMDTLASHQLYI | 349 |
| nNos | NP_434686 | 181 | rat | 1 | CAPON | NP_620277 | rat | LLNVLQRQELGDSLDDEIAV | 350 |
| nNos | NP_434686 | 181 | rat | 1 | PFK-M | NP_113903 | rat | TSDHAHLEHISRKRSGEAAV | 351 |
| CtpA | NP_442119 | 182 | Synecho- | 1 | D1-protein | NP_439906 | Synecho- | LDLASGEQAPVALTAPAVNG | 352 |
| | | | cystis | | | | cystis | | |
| GIPC | NP_001082286 | 183 | Xenopus | 1 | IGF1R | NP_001081734 | Xenopus | HMNGGRKNERALPLPQSSAC | 353 |
| GIPC | NP_001082286 | 183 | Xenopus | 1 | Frizzled-1 | NP_001079207 | Xenopus | NSWRKFYTRLTNSKQGETTV | 354 |

In some or any of the embodiments herein, a PDZ domain and PDZ-binding peptide pair is selected from Table 2. Table 2 lists three exemplary PDZ domains and respective PDZ-binding peptides. The PDZ-binding peptides for the three PDZ domains listed in Table 2 were isolated by screening a random library of putative PDZ-binding peptides via phage display as reported by Tonikian et al., PLos Biol 6:9 e239 (2008).

TABLE 2
PDZ Domains and screen-identified PDZ-binding peptides

| PDZ | | | | | | |
|---|---|---|---|---|---|---|
| containing | Accession | | | | | |
| protein | No. | Peptides* | | | | |
| ERBB2IP | NP_061165 | ISSSFFDTWV | SSFFRWDTWV | LLRLWMDTWV | KFPTFFDSWV | WHLSWFDDWV |
| | (SEQ ID | (SEQ ID NO: 355) | (SEQ ID NO: 374) | (SEQ ID NO: 393) | (SEQ ID NO: 412) | (SEQ ID NO: 431) |
| | NO: 102) | RHFIFFDTWV | LVRTSWDTWV | FVGSYADTWV | PRSSFFDSWV | NWLSWYDDWV |
| | | (SEQ ID NO: 356) | (SEQ ID NO: 375) | (SEQ ID NO: 394) | (SEQ ID NO: 413) | (SEQ ID NO: 432) |
| | | ITHLFFDTWV | LSFHCWDTWV | FLNRYRDTWV | SSQKFFDSWV | SCNYFYDEWV |
| | | (SEQ ID NO: 357) | (SEQ ID NO: 376) | (SEQ ID NO: 395) | (SEQ ID NO: 414) | (SEQ ID NO: 433) |
| | | RTSRWFDTWV | SASHFYDTWV | CSPSSIDTWV | RSPTFFDSWV | RSKCFLDEWV |
| | | (SEQ ID NO: 358) | (SEQ ID NO: 377) | (SEQ ID NO: 396) | (SEQ ID NO: 415) | (SEQ ID NO: 434) |
| | | NLGRYFDTWV | FGSRDYDTWV | SKGFWTDTWV | XXXXFFDSWV | LGSGWWDTFV |
| | | (SEQ ID NO: 359) | (SEQ ID NO: 378) | (SEQ ID NO: 397) | (SEQ ID NO: 416) | (SEQ ID NO: 435) |
| | | FPHPYFDTWV | RFDRSYDTWV | SFGAFVDTWV | GGHSWFDSWV | NSFCWWDTFV |
| | | (SEQ ID NO: 360) | (SEQ ID NO: 379) | (SEQ ID NO: 398) | (SEQ ID NO: 417) | (SEQ ID NO: 436) |
| | | SSYFPFDTWV | LITLFLDTWV | XXXXXPDTWV | SSGSWFDSWV | SSDSWYDTFV |
| | | (SEQ ID NO: 361) | (SEQ ID NO: 380) | (SEQ ID NO: 399) | (SEQ ID NO: 418) | (SEQ ID NO: 437) |
| | | SNRRHFDTWV | RCCFFLDTWV | HKPHFFETWV | RSWNYFDSWV | SNGRWYDTFV |
| | | (SEQ ID NO: 362) | (SEQ ID NO: 381) | (SEQ ID NO: 400) | (SEQ ID NO: 419) | (SEQ ID NO: 438) |
| | | SNLCHFDTWV | SVTHFLDTWV | HASSFFETWV | VRYSLFDSWV | MFSKFFDTWL |
| | | (SEQ ID NO: 363) | (SEQ ID NO: 382) | (SEQ ID NO: 401) | (SEQ ID NO: 420) | (SEQ ID NO: 439) |
| | | TKFHLFDTWV | TSFLTLDTWV | QLHSWFETWV | SRFLNFDSWV | QVSRFFETWL |
| | | (SEQ ID NO: 364) | (SEQ ID NO: 383) | (SEQ ID NO: 402) | (SEQ ID NO: 421) | (SEQ ID NO: 440) |
| | | GVHSVFDTWV | LFAVFNDTWV | HSSSYFETWV | RMSSRWDSWV | SPTSFFETWL |
| | | (SEQ ID NO: 365) | (SEQ ID NO: 384) | (SEQ ID NO: 403) | (SEQ ID NO: 422) | (SEQ ID NO: 441) |
| | | LPSRVFDTWV | SLPHYNDTWV | GSPPIFETWV | FSHSRWDSWV | STPFWFETWL |
| | | (SEQ ID NO: 366) | (SEQ ID NO: 385) | (SEQ ID NO: 404) | (SEQ ID NO: 423) | (SEQ ID NO: 442) |
| | | CRPGSFDTWV | WCSGRNDTWV | FTRSIFETWV | SRFWPWDSWV | LFGSFLETWL |
| | | (SEQ ID NO: 367) | (SEQ ID NO: 386) | (SEQ ID NO: 405) | (SEQ ID NO: 424) | (SEQ ID NO: 443) |
| | | KLRTGFDTWV | LLTQYDDTWV | SFAKWFQTWV | VSQFWRDSWV | LFSPWLETWL |
| | | (SEQ ID NO: 368) | (SEQ ID NO: 387) | (SEQ ID NO: 406) | (SEQ ID NO: 425) | (SEQ ID NO: 444) |
| | | LFLFFWDTWV | NLYMWDDTWV | PVIWLFHTWV | LPFPFHDSWV | RSVSWIETWL |
| | | (SEQ ID NO: 369) | (SEQ ID NO: 388) | (SEQ ID NO: 407) | (SEQ ID NO: 426) | (SEQ ID NO: 445) |
| | | ISSSFFDTWV | SSFFRWDTWV | LLRLWMDTWV | KFPTFFDSWV | WHLSWFDDWV |
| | | (SEQ ID NO: 370) | (SEQ ID NO: 389) | (SEQ ID NO: 408) | (SEQ ID NO: 427) | (SEQ ID NO: 446) |
| | | MPNSFWDTWV | HPLTDSDTWV | RLDRFLATWV | SHTCWHDSWV | |
| | | (SEQ ID NO: 371) | (SEQ ID NO: 390) | (SEQ ID NO: 409) | (SEQ ID NO: 428) | |
| | | IPPMYWDTWV | SSANGSDTWV | FCRRWFDSWV | LCKAWFESWV | |
| | | (SEQ ID NO: 372) | (SEQ ID NO: 391) | (SEQ ID NO: 410) | (SEQ ID NO: 429) | |
| | | GSYFRWDTWV | LYGGFSDTWV | VTPSFFDSWV | NDRMWFDDWV | |
| | | (SEQ ID NO: 373) | (SEQ ID NO: 392) | (SEQ ID NO: 411) | (SEQ ID NO: 430) | |
| SCRIB | NP_056171 | YLQRFLETHL | LHMLWRETHL | FSSIWRETDL | RCAGYCETSL | |
| | (SEQ ID | (SEQ ID NO: 447) | (SEQ ID NO: 467) | (SEQ ID NO: 487) | (SEQ ID NO: 507) | |
| | NO: 548) | VMRYFLETHL | XASVYRETHL | MHRIWHETDL | RRLPWFETRL | |
| | | (SEQ ID NO: 448) | (SEQ ID NO: 468) | (SEQ ID NO: 488) | (SEQ ID NO: 508) | |
| | | SLRGFLETHL | LLRCWKETHL | RSLPYHETDL | VHLLWRETRL | |
| | | (SEQ ID NO: 449) | (SEQ ID NO: 469) | (SEQ ID NO: 489) | (SEQ ID NO: 509) | |
| | | RTISFLETHL | HSSVWMETHL | RWRPWNETDL | TSKVWRETRL | |
| | | (SEQ ID NO: 450) | (SEQ ID NO: 470) | (SEQ ID NO: 490) | (SEQ ID NO: 510) | |
| | | RSVSFLETHL | MNRFFNETHL | RGTFWSETDL | IRRRWSETFL | |
| | | (SEQ ID NO: 451) | (SEQ ID NO: 471) | (SEQ ID NO: 491) | (SEQ ID NO: 511) | |
| | | RKSFFLETHL | SLRRFLETDL | RRLGFCETDL | LFRRFLETNL | |
| | | (SEQ ID NO: 452) | (SEQ ID NO: 472) | (SEQ ID NO: 492) | (SEQ ID NO: 512) | |
| | | AISYFLETHL | TPRGFLETDL | RHAHYLETWL | ASRIWRETDI | |
| | | (SEQ ID NO: 453) | (SEQ ID NO: 473) | (SEQ ID NO: 493) | (SEQ ID NO: 513) | |
| | | LLRVFLETHL | SRRSFLETDL | RHTTYFETWL | RASYREGDWI | |
| | | (SEQ ID NO: 454) | (SEQ ID NO: 474) | (SEQ ID NO: 494) | (SEQ ID NO: 514) | |
| | | LFRVFLETHL | RRVDFLETDL | FSHYREGDWL | LLRIWRETSI | |
| | | (SEQ ID NO: 455) | (SEQ ID NO: 475) | (SEQ ID NO: 495) | (SEQ ID NO: 515) | |
| | | CLSSWLETHL | RLSRFLETDL | TSHYSEGDWL | IRRIWRETSM | |
| | | (SEQ ID NO: 456) | (SEQ ID NO: 476) | (SEQ ID NO: 496) | (SEQ ID NO: 516) | |
| | | ARDNWLETHL | RAAGFLETDL | RASYKQGDWL | LLRVFRETSM | |
| | | (SEQ ID NO: 457) | (SEQ ID NO: 477) | (SEQ ID NO: 497) | (SEQ ID NO: 517) | |
| | | SLIRYLETHL | RCFGFLETDL | RSSYRVGDWL | | |
| | | (SEQ ID NO: 458) | (SEQ ID NO: 478) | (SEQ ID NO: 498) | | |
| | | RFLGLLETHL | KFLRFLETDL | RASYKPGDWL | | |
| | | (SEQ ID NO: 459) | (SEQ ID NO: 479) | (SEQ ID NO: 499) | | |
| | | TFRSFFETHL | YVRWYLETDL | LLRRYLETSL | | |
| | | (SEQ ID NO: 460) | (SEQ ID NO: 480) | (SEQ ID NO: 500) | | |
| | | FVSIFFETHL | RVSSYLETDL | ILSRFFETSL | | |
| | | (SEQ ID NO: 461) | (SEQ ID NO: 481) | (SEQ ID NO: 501 | | |
| | | FLKRFFETHL | NRFPYLETDL | RFLRFFETSL | | |
| | | (SEQ ID NO: 462) | (SEQ ID NO: 482) | (SEQ ID NO: 502) | | |
| | | SLTRFFETHL | SSRRFFETDL | IRRAYFETSL | | |
| | | (SEQ ID NO: 463) | (SEQ ID NO: 483) | (SEQ ID NO: 503) | | |
| | | RGQSFFETHL | NRPRFFETDL | LGRAWRETSL | | |
| | | (SEQ ID NO: 464) | (SEQ ID NO: 484) | (SEQ ID NO: 504) | | |
| | | NSRSYFETHL | SKSCYFETDL | LARLWRETSL | | |
| | | (SEQ ID NO: 465) | (SEQ ID NO: 485) | (SEQ ID NO: 505) | | |
| | | SIRVWRETHL | NVHRFYETDL | SSGCYRETSL | | |
| | | (SEQ ID NO: 466) | (SEQ ID NO: 486) | (SEQ ID NO: 506) | | |
| NTRA1 | NP_002766 | LWLGWKTWIL | | | | |
| | (SEQ ID | (SEQ ID NO: 518) | | | | |
| | NO: 549) | XXEKSKIWFV | | | | |
| | | (SEQ ID NO: 519) | | | | |
| | | RWKDIETWLL | | | | |
| | | (SEQ ID NO: 520) | | | | |
| | | KSEDSRIWWV | | | | |
| | | (SEQ ID NO: 521) | | | | |
| | | QDVPGKIWFV | | | | |
| | | (SEQ ID NO: 522) | | | | |
| | | LCRVYECFWL | | | | |
| | | (SEQ ID NO: 523) | | | | |
| | | NSRLWDRDVF | | | | |
| | | (SEQ ID NO: 524) | | | | |
| | | KMLWDKIWHV | | | | |
| | | (SEQ ID NO: 525) | | | | |
| | | LCHIFKCFLV | | | | |
| | | (SEQ ID NO: 526) | | | | |
| | | LCRRFFCFYL | | | | |
| | | (SEQ ID NO: 527) | | | | |
| | | RDTIWEIFHF | | | | |
| | | (SEQ ID NO: 528) | | | | |
| | | RGGIDRIWWV | | | | |
| | | (SEQ ID NO: 529) | | | | |
| | | XXXXXKVGGF | | | | |
| | | (SEQ ID NO: 530) | | | | |
| | | LGDIDKSCRV | | | | |
| | | (SEQ ID NO: 531) | | | | |
| | | LPRIWEIWTL | | | | |
| | | (SEQ ID NO: 532) | | | | |
| | | FRLWDVIWLV | | | | |
| | | (SEQ ID NO: 533) | | | | |

*X represents any of the 20 naturally occurring amino acids

A person of ordinary skill in the art would appreciate that the methods disclosed by Tonikian et al. are useful to generate novel PDZ domain/PDZ-binding peptide pairs useful in the materials and methods disclosed herein. Tonikian et al. describe four PDZ domains that recognize PDZ-binding peptides with cysteines in the 0 position, including APBA3-1 (human amyloid beta A4 precursor protein-binding family A member 3, Accession no. NP_{—}004877), T21G5.4-1 (C. elegans hypothetical protein, Accession no. AAB2899), C25G4.6-2 (C. elegans hypothetical protein, Accession no. NP_{—}502380), and C53B4.4 (C. elegans hypothetical protein, Accession no. NP_{—}001122764). The APBA3-1, T21G5.4-1, C25G4.6-2, and C53B4.4 PDZ domains bind to the consensus sequence FDΩΩ C (SEQ ID NO: 534) wherein Ω is an aromatic amino acid (F, W, or Y). A person of ordinary skill in the art would appreciate that the PDZ domains shown to preferentially bind PDZ-binding peptides with cysteine residues at the 0 position could be engineered to comprise a cysteine residue that forms a disulfide bond with the aforementioned PDZ-binding peptide cysteine. Moreover, a person of ordinary skill in the art would appreciate that structural information could be used to engineer PDZ domain/PDZ-binding peptide pairs comprising disulfide bond-forming cysteine residues similar to the naturally occurring InaD PDZ1 domain/NorpA pair described above. In certain embodiments, a PDZ domain is engineered to replace a native amino acid residue with a cysteine in the peptide binding groove of the PDZ domain or a region outside the peptide binding groove. In other embodiments, a PDZ-binding peptide is engineered to replace a native amino acid residue with a cysteine residue at the C-terminus of a PDZ-binding peptide or a region outside the C-terminal region. It is also contemplated that in some embodiments, a PDZ binding domain and a PDZ-binding peptide are engineered to replace native residues with cysteine residues in order to generate a PDZ domain/PDZ-binding peptide linked by a disulfide bond. PDZ domain/PDZ-binding peptide pairs capable of disulfide bonding are advantageous in that the PDZ domain/PDZ-binding peptide interaction is more stable due to the fact that it is covalent.

### C. HOST CELLS AND CELL SURFACE PROTEINS

As used herein “cell surface proteins” are naturally occurring proteins or portions thereof that are displayed on the surface of cells, or fragments or variants thereof that retain the ability to be displayed on the cell surface.

1. Yeast

In some or any embodiments, the yeast strain is from a genus selected from the group consisting of Saccharomyces, Pichia, Hansenula, Schizosaccharomyces, Kluyveromyces, Yarrowia, and Candida. In some or any embodiments, the yeast species is selected from the group consisting of S. cerevisiae, P. pastoris, H. polymorpha, S. pombe, K. lactis, Y. lipolytica, and C. albicans.

In some or any embodiments, the yeast strain has been engineered to carry out glycosylation reactions of the type performed in human cells. Exemplary methods for glycoengineering of yeast are reviewed in Nat Rev Microbiol 3 (2):119-28 (2005).

The methods and cells of the invention provide PDZ Domains fused to a cell wall protein to enable protein display on the surface of cells. When the host cell is a yeast cell, any suitable cell wall protein may be fused to the PDZ Domain. When the host cell is S. cerevisiae, examples of suitable cell wall proteins include Aga1, Aga2, Aga1, Cwp1, Cwp2, Gas1p, Yap3p, Flo1p, Crh2p, Pir1, Pir2, Pir3, or Pir4, or fragments or variants thereof. When the host cell is H. polymorpha, examples of suitable cell wall proteins include HpSED1, HpGAS1, HpTIP1, or HPWP1. When the host cell is C. albicans, examples of suitable cell wall proteins include Hwp1p, Als3p, or Rbt5p. In example embodiments, the fragments of such cell wall proteins are at least about 20, 25, 30, 35, 40, 45, or 50 amino acids in length. In example embodiments, the variants thereof comprise an amino acid sequence at least 80%, 85%, 90% or 95% identical to at least 100 amino acids of such domains.

2. Mammalian Cells

It is also contemplated that the methods disclosed herein are carried out using mammalian host cells. Examples of useful mammalian host cell lines are Chinese hamster ovary cells, including CHOK1 cells (ATCC CCL61), DXB-11, DG-44, Chinese hamster ovary cells/−DHFR(CHO, Urlaub et al., Proc. Natl. Acad. Sci. USA 77: 4216 (1980)), and CHO cells engineered to produce controlled fucosylation (MAbs. 1(3):230-36 (2009)); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, (Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383: 44-68 (1982)); MRC 5 cells; FS4 cells; and a human hepatoma line (Hep G2).

When the host cell is a mammalian cell, examples of portions of cell surface proteins that retain the ability to display proteins on the cell surface include suitable transmembrane domain of any known cell membrane proteins, or a polypeptide with a GPI anchor sequence, or a non-cleavable type II signal anchor sequence. Examples of membrane anchor sequences used for cell display in mammalian cells include PDGFR transmembrane domain (Chesnut et al., J Immunol Methods 193(1): 17-27, (1996); Ho et al., Proc Natl Acad Sci USA 103(25): 9637-42, (2006); incorporated by reference in their entirety), GPI anchor from human decay-accelerating factor (Akamatsu et al., J Immunol Methods, 327(1-2): 40-52 (2007); incorporated by reference in its entirety) and T-cell receptor (TCR) chain (Alonso-Camino et al., PLoS One 4(9): e7174 (2009); incorporated by reference in its entirety). Another example is the use of type II signal anchor sequences (U.S. Pat. No. 7,125,973; incorporated by reference in its entirety). Alternatively, a capture molecule such as an antibody or protein can be fused to a membrane anchor sequence, and displayed on the cell surface in order to capture the protein of interest (U.S. Pat. No. 6,919,183; incorporated by reference in its entirety). In certain embodiments, an artificial cell surface anchor sequence is assembled into, or attached to, the cell membrane of mammalian cells.

3. Prokaryotes

It is also contemplated that the methods disclosed herein are carried out using prokaryotic host cells. Thus, in some or any embodiments, the host cell is a prokaryotic cell. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformnis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

When the host cell is a prokaryotic cell, examples of suitable cell surface proteins include suitable bacterial outer membrane proteins. Such outer membrane proteins include pili and flagella, lipoproteins, ice nucleation proteins, and autotransporters. Exemplary bacterial proteins used for heterologous protein display include LamB (Charbit et al., EMBO J, 5(11): 3029-37 (1986); incorporated by reference in its entirety), OmpA (Freudl, Gene, 82(2): 229-36 (1989); incorporated by reference in its entirety) and intimin (Wentzel et al., J Biol Chem, 274(30): 21037-43, (1999); incorporated by reference in its entirety). Additional exemplary outer membrane proteins include, but are not limited to, FliC, pullulunase, OprF, OprI, PhoE, M isL, and cytolysin. An extensive list of bacterial membrane proteins that have been used for surface display and are contemplated for use in the present invention are detailed in Lee et al., Trends Biotechnol, 21(1): 45-52 (2003), Jose, Appl Microbiol Biotechnol, 69(6): 607-14 (2006), and Daugherty, Curr Opin Struct Biol, 17(4): 474-80 (2007), all incorporated by reference in their entirety. In certain embodiments, the anchor protein is an artificial sequence that is assembled into, or attaches to the outer surface of the bacterial cell.

### D. POLYPEPTIDE-BINDING AGENTS

In some or any embodiments, the protein of interest is a polypeptide binding agent. The term “polypeptide binding agent,” as used herein, refers to a polypeptide that is capable of specifically binding another molecular entity (e.g., an antigen), or that is capable of binding another molecular entity with a measurable binding affinity. Examples of polypeptide binding agents include antibodies, peptibodies, proteases, scaffold proteins, polypeptides and peptides, optionally conjugated to other peptide moieties or non-peptidic moieties. Molecular entities to which a polypeptide binding agent may bind include any proteinaceous or non-proteinaceous molecule that is capable of eliciting an antibody response, or that is capable of binding to a polypeptide binding agent with detectable binding affinity greater than non-specific binding.

In example embodiments, the polypeptide binding agent is an antibody. The term “antibody” is used in the broadest sense and includes fully assembled antibodies, tetrameric antibodies, monoclonal antibodies, polyclonal antibodies, multispecific antibodies (e.g., bispecific antibodies, mAb² antibodies,), antibody fragments that can bind an antigen (e.g., Fab′, F′(ab)₂, Fv, single chain antibodies, diabodies, dAbs), and recombinant peptides comprising the forgoing as long as they exhibit the desired biological activity. An “immunoglobulin” or “tetrameric antibody” is a tetrameric glycoprotein that consists of two heavy chains and two light chains, each comprising a variable region and a constant region. Antigen-binding portions may be produced by recombinant DNA techniques or by enzymatic or chemical cleavage of intact antibodies. Antibody fragments or antigen-binding portions include, inter alia, Fab, Fab′, F(ab′)2, Fv, domain antibody (dAb), Fcab™, complementarity determining region (CDR) fragments, single-chain antibodies (scFv), single chain antibody fragments, antibody molecules containing just two CDRs linked by a framework region, e.g., V_{H}CDR1-V_{H}FR2-V_{L}CDR3 fusion peptides, chimeric antibodies, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, a camelized antibody, a VHH-containing antibody, or a variant or a derivative thereof, and polypeptides that contain at least a portion of an immunoglobulin that is sufficient to confer specific antigen binding to the polypeptide, such as one, two, three, four, five, or six CDR sequences, as long as the antibody retains the desired biological activity.

In a naturally-occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa (κ) and lambda (λ) light chains. Heavy chains are classified as mu (μ), delta (Δ), gamma (γ), alpha (α), and epsilon (ε), and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology, Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains (Chothia et al., J. Mol. Biol. 196:901-917, 1987).

Immunoglobulin variable domains exhibit the same general structure of relatively conserved framework regions (FR) joined by three hypervariable regions or CDRs. From N-terminus to C-terminus, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk, (J. Mol. Biol. 196:901-917, 1987); Chothia et al., (Nature 342:878-883, 1989).

The hypervariable region of an antibody refers to the CDR amino acid residues of an antibody which are responsible for antigen-binding. The hypervariable region comprises amino acid residues from a CDR (residues 24-34 (L1), 50-56 (L2) and 89-97 (L3) in the light chain variable domain and 31-35 (H1), 50-65 (H2) and 95-102 (H3) in the heavy chain variable domain as described by Kabat et al., Sequences of Proteins of Immunological Interest, 5th Ed. Public Health Service, National Institutes of Health, Bethesda, Md. (1991)) and/or those residues from a hypervariable loop (residues 26-32 (L1), 50-52 (L2) and 91-96 (L3) in the light chain variable domain and 26-32 (H1), 53-55 (H2) and 96-101 (H3) in the heavy chain variable domain as described by Chothia et al., J. Mol. Biol. 196: 901-917 (1987).

Framework or FR residues are those variable domain residues other than the hypervariable region residues.

“Heavy chain variable region” as used herein refers to the region of the antibody molecule comprising at least one complementarity determining region (CDR) of said antibody heavy chain variable domain. The heavy chain variable region may contain one, two, or three CDRs of said antibody heavy chain.

“Light chain variable region” as used herein refers to the region of an antibody molecule, comprising at least one complementarity determining region (CDR) of said antibody light chain variable domain. The light chain variable region may contain one, two, or three CDRs of said antibody light chain, which may be either a kappa or lambda light chain depending on the antibody.

Depending on the amino acid sequence of the constant domain of their heavy chains, immunoglobulins can be assigned to different classes, IgA, IgD, IgE, IgG and IgM, which may be further divided into subclasses or isotypes, e.g. IgG1, IgG2, IgG3, IgG4, IgA1 and IgA2. The subunit structures and three-dimensional configurations of different classes of immunoglobulins are well known. Different isotypes have different effector functions; for example, IgG1 and IgG3 isotypes have ADCC activity. An antibody of the invention, if it comprises a constant domain, may be of any of these subclasses or isotypes, or a variant or consensus sequence thereof, or a hybrid of different isotypes (e.g., IgG1/IgG2 hybrid).

In exemplary embodiments, an antibody of the invention can comprise a human kappa (κ) or a human lambda (λ) light chain or an amino acid sequence derived therefrom, or a hybrid thereof, optionally together with a human heavy chain or a sequence derived therefrom, or both heavy and light chains together in a single chain, dimeric, tetrameric (e.g., two heavy chains and two light chains) or other form.

“Monoclonal antibody” refers to an antibody obtained from a population of substantially homogeneous antibodies, i.e., the individual antibodies comprising the population are identical except for possible naturally occurring mutations that may be present in minor amounts.

“Antibody variant” as used herein refers to an antibody polypeptide sequence that contains at least one amino acid substitution, deletion, or insertion in the variable region of the natural antibody variable region domains. Variants may be substantially homologous or substantially identical to the unmodified antibody.

A “chimeric antibody,” as used herein, refers to an antibody containing sequence derived from two different antibodies (see, e.g., U.S. Pat. No. 4,816,567) which typically originate from different species. Most typically, chimeric antibodies comprise human and rodent antibody fragments, generally human constant and mouse variable regions.

A “neutralizing antibody” is an antibody molecule which is able to eliminate or significantly reduce a biological function of an antigen to which it binds. Accordingly, a “neutralizing” antibody is capable of eliminating or significantly reducing a biological function, such as enzyme activity, ligand binding, or intracellular signaling.

An “isolated” antibody is one that has been identified and separated and recovered from a component of its natural environment. Contaminant components of its natural environment are materials that would interfere with diagnostic or therapeutic uses for the antibody, and may include enzymes, hormones, and other proteinaceous or non-proteinaceous solutes. In some embodiments, the antibody is purified, e.g., (1) to greater than 95% by weight of antibody as determined by the Lowry method, and preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

In other example embodiments the polypeptide binding agent is a protease. The term “protease” as used herein refers to any protein molecule catalyzing the hydrolysis of peptide bonds. It includes naturally-occurring proteolytic enzymes, as well as protease variants. It also comprises any fragment of a proteolytic enzyme, or any molecular complex or fusion protein comprising one of the aforementioned proteins. Proteases include, but are not limited to: trypsin, chymotrypsin, substilisin, thrombin, plasmin, Factor Xa, uPA, tPA, MTSP-1, granzyme A, granzyme B. granzyme M, elastase, chymase, papain, neutrophil elastase, plasma kallikrein, urokinase type plasminogen activator, complement factor serine proteases, ADAMTS 13, neural endopeptidase/neprilysin, furin, and cruzain.

In further example embodiments the polypeptide binding agent is a scaffold. Protein scaffolds include, but are not limited to, AdNectins, Affibodies, Anticalins, DARPins, engineered Kunitz-type inhibitors, tetranectins, A-domain proteins, lipocalins, repeat proteins such as ankyrin repeat proteins, immunity proteins, α2p8 peptide, insect defensin A, PDZ domains, charybdotoxins, PHD fingers, TEM-1 β-lactamase, fibronectin type III domains, CTLA-4, T-cell receptors, knottins, neocarzinostatin, carbohydrate binding module 4-2, green fluorescent protein, thioredoxin (Gebauer & Skerra, Curr. Opin. Chem. Biol. 13:245-55 (2009); Gill & Damle, Curr. Opin. Biotech 17: 653-58 (2006); Hosse et al, Protein Sci. 15:14-27 (2006); Skerra, Curr. Opin. Biotech 18: 295-3-4 (2007)).

### E. VECTORS

The vectors of the present invention generally comprise transcriptional or translational control sequences required for expressing the exogenous polypeptide. Suitable transcription or translational control sequences include but are not limited to replication origin, promoter, enhancer, repressor binding regions, transcription initiation sites, ribosome binding sites, translation initiation sites, and termination sites for transcription and translation.

In some or any embodiments, the polynucleotides encoding a cell surface protein fused to a PDZ Domain and a protein of interest (e.g., polypeptide binding agent or antibody or antigen-binding fragment thereof) fused to a PDZ-binding peptide are present on the same vector. In some or any embodiments, the polynucleotides encoding a cell surface protein fused to a PDZ Domain and a protein of interest (e.g., polypeptide binding agent or antibody or antigen-binding fragment thereof) fused to a PDZ-binding peptide are present on different vectors. As will be appreciated by a person of skill in the art, each fusion-encoding polynucleotide will have suitable transcription and translational control sequences and signal sequences to allow for appropriate expression in the host cell.

In some or any embodiments, the polynucleotides encoding a cell surface protein fused to a PDZ Domain are integrated into the genome of the host cell. When the host cell is a yeast cell, yeast integrative plasmids (YIp) may be used to integrate the polynucleotides into the yeast cell genome. The site of integration can be targeted by cutting the yeast segment in the YIp plasmid with a restriction endonuclease and transforming the yeast strain with the linearized plasmid. The linear ends are recombinogenic and direct integration to the site in the genome that is homologous to these ends. In addition, linearization increases the efficiency of integrative transformation from 10- to 50-fold. Strains transformed with YIp plasmids are extremely stable, even in the absence of selective pressure.

In some or any embodiments of the invention, the expression vector is a shuttle vector, capable of replicating in at least two unrelated expression systems. In order to facilitate such replication, the vector generally contains at least two origins of replication, one effective in each expression system. Shuttle vectors may be capable of replicating in a eukaryotic expression system and a prokaryotic expression system. Alternatively, shuttle vectors may be capable of replicating in two different eukaryotic systems, for example in yeast and in mammalian systems, or in two different prokaryotic systems. This enables detection of protein expression in the eukaryotic host and amplification of the vector in the prokaryotic host. In one embodiment, one origin of replication is a CEN ori and one is derived from pUC although any suitable origin known in the art may be used provided it directs replication of the vector. Where the vector is a shuttle vector, the vector contains at least two selectable markers, for example, one for a eukaryotic cell and one for a prokaryotic cell. Any selectable marker known in the art or those described herein may be used provided it functions in the expression system being utilized.

1. Origins of Replication

The origin of replication (generally referred to as an ori sequence) permits replication of the vector in a suitable host cell. The choice of ori will depend on the type of host cells that are employed. Where the host cells are prokaryotes, the expression vector typically comprises an ori directing autonomous replication of the vector within the prokaryotic cells. Non-limiting examples of this class of ori include pMB1, pUC, as well as other bacterial origins.

Higher eukaryotes contain multiple origins of DNA replication (estimated 104-106 ori/mammalian genome), but the ori sequences are not so clearly defined. The suitable origins for mammalian vectors are normally from eukaryotic viruses. Exemplary eukaryotic ori sequences include, but are not limited to, SV40 ori, EBV ori, and HSV oris. Exemplary ori sequences for yeast cells include, but are not limited to, 2 μm ori sequences and CEN ori sequences.

2. Signal Sequences

Signal sequences from both prokaryotes and eukaryotes share several common characteristics. They are about 15-30 amino acids in length and consist of three regions: a positively charged N-terminal region, a central hydrophobic region, and a more polar C-terminal region. When the host cell is a yeast cell, any signal sequence known in the art capable of directing a protein to be secreted and/or directed to the cell wall can be used. For example, the signal sequence can be derived from Mating Factor α1 (MFα1) (Bitter et al., Proc Natl Acad Sci USA 81(17): 5330-4 (1984)), Invertase (SUC2) (Taussig and Carlson, Nucleic Acids Res 11(6): 1943-54 (1983)), Acid phosphatase (PHOS) (Arima et al., Nucleic Acids Res 11(6): 1657-72 (1983)), Beta glucanase (BGL2) (Achstetter et al., Gene 110(1): 25-31 (1992)), and Inulinase (INU1A) (Chung et al., Biotechnol Bioeng 49(4): 473-9 (1996)). The signal sequence of yeast GPI proteins such as AGA2, AGA1, AGα1, FLO1, GAS1, CWP1, and CWP2 that are covalently linked to the cell wall and have been shown to be compatible for cell surface protein display are also within the scope of the invention (De Groot et al., Yeast 20(9): 781-96 (1992)).

When the host cell is a prokaryotic cell, signal sequences directing fusion polypeptides for periplasmic secretion include those derived from spA, phoA, ribose binding protein, pelB, ompA, ompT, dsbA, torA, torT, and tolT (de Marco, Microbial Cell Factories, 8:26 (2009)). The pelB signal sequences disclosed in U.S. Pat. Nos. 5,846,818 and 5,576,195 are incorporated by reference in their entirety.

Also included within the scope of the invention are signal sequences derived from eukaryotic cells that also function as signal sequences in prokaryotic host cells (e.g., E. coli). Such sequences are disclosed in U.S. Pat. No. 7,094,579, the content of which is incorporated by reference in its entirety.

Watson (Nucleic Acids Research 12:5145-5164 (1984)) discloses a compilation of signal sequences. U.S. Pat. No. 4,963,495 discloses the expression and secretion of mature eukaryotic protein in the periplasmic space of a host organism using a prokaryotic secretion signal sequence DNA linked at its 3′ end to the 5′ end of the DNA encoding the mature protein. Chang et al. (Gene 55:189-196 (1987)) discloses the use of the STII signal sequence to secrete hGH in E. coli. Gray et al. (Gene 39:247-245 (1985)) disclose the use of the natural signal sequence of human growth hormone and the use of the E. coli alkaline phosphatase promoter and signal sequence for the secretion of human growth hormone in E. coli. Wong et al. (Gene 68:193-203 (1988)) disclose the secretion of insulin-like growth factor 1 (IGF-1) fused to LamB and OmpF secretion leader sequences in E. coli, and the enhancement of processing efficiency of these signal sequences in the presence of a pr1A4 mutation. Fujimoto et al. (J. Biotech. 8:77-86 (1988)) disclose the use of four different E. coli enterotoxin signal sequences, STI, STII, LT-A, and LT-B for the secretion of human epidermal growth factor (hEGF) in E. coli. Denefle et al. (Gene 85:499-510 (1989)) disclose the use of OmpA and PhoA signal peptides for the secretion of mature human interleukin 1β. Content of all of the above documents is incorporated by reference in its entirety.

3. Promoters

Suitable promoter sequences for eukaryotic cells include the promoters for 3-phosphoglycerate kinase, or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other promoters, which have the additional advantage of transcription controlled by growth conditions, are the promoter regions for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Preferred promoters for mammalian cells are SV40 promoter, CMV promoter, β-actin promoter and their hybrids. Preferred promoters for yeast cells include but are not limited to GAL10, GAL1, TEF1, CUP1, ADH2, GPD in S. cerevisiae, and GAP, AOX1 in P. pastoris. A variety of robust prokaryotic promoters are known in the art. Preferred promoters are lac promoter, Trc promoter, T7 promoter and pBAD promoter.

4. Terminators

The terminator sequence preferably contains one or more transcriptional termination sequences (such as polyadenylation sequences) and may also be lengthened by the inclusion of additional DNA sequence so as to further disrupt transcriptional read-through. Preferred terminator sequences (or termination sites) of the present invention have a gene that is followed by a transcription termination sequence, either its own termination sequence or a heterologous termination sequence. Examples of such termination sequences include stop codons coupled to various yeast transcriptional termination sequences or mammalian polyadenylation sequences that are known in the art and widely available.

5. Selectable Markers

In addition to the above-described elements, the vectors may contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will survive and/or grow under selective conditions. Typical selection genes encode protein(s) that (a) confer resistance to antibiotics or other toxins, e.g., ampicillin, kanamycin, neomycin, G418, methotrexate, etc.; (b) complement auxotrophic deficiencies; or (c) supply critical nutrients not available from complex media. The choice of the proper marker gene will depend on the host cell, and appropriate genes for different hosts are known in the art.

The vectors encompassed by the invention can be obtained using recombinant cloning methods and/or by chemical synthesis. A vast number of recombinant cloning techniques such as PCR, restriction endonuclease digestion and ligation are well known in the art. One of skill in the art can also use the sequence data provided herein or that in the public or proprietary databases to obtain a desired vector by any synthetic means available in the art. Additionally, using well-known restriction and ligation techniques, appropriate sequences can be excised from various DNA sources and integrated in operative relationship with the exogenous sequences to be expressed in accordance with the present invention.

### F. METHODS OF PREPARING AND SCREENING LIBRARIES

In some or any embodiments, a library of cells comprising at least 10̂3, at least 10̂4, at least 10̂5, at least 10̂6, at least 10̂7, at least 10̂8, at least 10̂9, or at least 10̂ different host cells, e.g., yeast cells, each such host cell displaying on its surface a different protein of interest (e.g., polypeptide binding agent, antibody, or antigen-binding fragment thereof), is contemplated. Display of the protein of interest, e.g., antibody, or antigen-binding fragment thereof, is accomplished by the expression of fusion proteins utilizing the interaction between a PDZ Domain and a PDZ-binding peptide, as described herein. Methods of generating host cells comprising a library of antibodies or antigen-binding portions thereof, are known in the art and described herein. Such cell libraries are screened using methods known in the art and described herein (such as FACS or MACS) to identify antibodies or antigen-binding fragments thereof that bind target proteins/antigens.

The invention contemplates methods of producing target-specific antibody or antigen-binding portion thereof comprising creating a library of antibodies or antigen-binding fragments displayed on a cell surface. Libraries of antibodies or antigen-binding fragments may be prepared from immunized or non-immunized sources, and may be natural, semi-synthetic or synthetic (reviewed in Hoogenboom, Nat. Biotech. 23(9): 1105-1116 (2005)).

The invention also contemplates methods of identifying target-specific antibody or antigen-binding portion thereof comprising contacting the library with target protein or a portion thereof, selecting or isolating or sorting cell(s) that bind target, and obtaining the antibody or antigen-binding fragment thereof from the cell(s). Examples of methods for selection are described below under “Cell Sorting.”

By way of example, a method for preparing the library of antibodies or antigen-binding fragments for use in cell surface display methods disclosed herein comprises the steps of immunizing a non-human animal comprising human immunoglobulin loci with target antigen or an antigenic portion thereof to create an immune response, extracting antibody producing cells from the immunized animal; isolating RNA from the extracted cells, reverse transcribing the RNA to produce cDNA, amplifying the cDNA, and inserting the cDNA into the display vectors disclosed herein such that antibodies are expressed on the cell surface of a host cell. Methods for constructing and screening an antibody library have been described in Winter et al., PCT Publication No. WO 90/05144, and U.S. Pat. No. 6,057,098 (which are incorporated herein by reference).

By way of another example, a method for preparing the library of antibodies or antigen-binding fragments for use in cell surface display methods disclosed herein comprises the steps of isolating mRNA from animal, e.g. human, spleen cells or peripheral blood lymphocytes, reverse transcribing the RNA to produce cDNA, amplifying the cDNA, and inserting the cDNA into the display vectors disclosed herein such that antibodies are expressed on the cell surface of a host cell. Alternatively the libraries of different nucleotide sequence may be derived by the in vitro mutagenesis of an existing antibody-coding sequence.

By way of a further example, libraries of protease variants for use in cell surface display methods disclosed herein may be prepared according to the methods described in WO/04031733 and WO/06125827 (which are incorporated herein by reference). Different strategies of introducing changes in the coding sequences include, but are not limited to, single or multiple point mutations, exchange of single or multiple nucleotide triplets, insertions or deletions of one or more codons, homologous or heterologous recombination between different genes, fusion of additional coding sequences at either end of the encoding sequence or insertion of additional encoding sequences or any combination of these methods.

In some or any embodiments, a library of proteins of interest is subcloned from an existing display library, e.g. a phage display sub-library created by one or more rounds of panning against an antigen. For example, WO/9847343 describes methods of subcloning nucleic acids encoding displayed polypeptides of enriched libraries from a display vector to an expression vector to produce polyclonal libraries of antibodies and other polypeptides. By way of further example, Jostock et al, J. Immunol. Methods 289, 65-80 (2004) describes batch reformatting of Fab fragments in a phage vector to IgGs in a mammalian vector.

Methods of screening the libraries are described in the Examples. Additional methods and reagents that can be used in generating and screening antibody display libraries are available in the art (see, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum. Antibod. Hybridomas 3:81-85; Huse et al. (1989) Science 246:1275-1281; McCafferty et al., Nature (1990) 348:552-554; Griffiths et al. (1993) EMBO J. 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nuc Acid Res 19:4133-4137; and Barbas et al. (1991) Proc. Natl. Acad. Sci. USA 88:7978-7982.

### G. CELL SORTING

Flow cytometry is a powerful, high-throughput library screening tool with numerous applications including the isolation of bioactive molecules from synthetic combinatorial libraries, the identification of virulence genes in microorganisms, and the study and engineering of protein functions. Using flow cytometry, large libraries of protein mutants expressed in microorganisms can be screened quantitatively for desired functions, including ligand binding, catalysis, expression level, and stability. Rare target cells, occurring at frequencies below 10⁻⁶, can be detected and isolated from heterogeneous library populations using one or more cycles of cell sorting and amplification by growth. Flow cytometry is particularly powerful because it provides the unique opportunity to observe and quantitatively optimize the screening process. However, the ability to isolate cells occurring at such low frequencies within a population requires consideration and optimization of screening parameters.

Libraries of cells displaying antibody fragments on the surface are screened for antigen binding by either magnetic activated cell sorting (MACS) or fluorescent activated cell sorting (FACS). FACS employs a plurality of color channels, low angle and obtuse light-scattering detection channels, and impedance channels, among other more sophisticated levels of detection, to separate or sort cells. Any FACS technique may be employed as long as it is not detrimental to the viability of the desired cells. (For exemplary methods of FACS, see U.S. Pat. No. 5,061,620). For libraries >10⁸ in size, typically MACS is used to reduce the size of the library in order to allow subsequent screening by FACS to be done in a feasible time period. In MACS, members of the library that bind biotin labeled antigen are isolated using streptavidin-coated magnetic beads and magnetic separation, then propagated for additional screening. In FACS, simultaneous assessment of antigen binding and antibody expression using two-color detection permits the identification of a population of high affinity clones which are then propagated for subsequent rounds of screening.

Separation procedures may include magnetic separation, using antigen-coated magnetic beads and “panning,” which utilizes an antigen attached to a solid matrix. Antigens attached to magnetic beads and other solid matrices, such as agarose beads, polystyrene beads, hollow fiber membranes and plastic petri dishes, allow for direct separation. Cells that are bound by the antigen can be removed from the cell suspension by simply physically separating the solid support from the cell suspension. The exact conditions and duration of incubation of the cells with the solid phase-linked antigens will depend upon several factors specific to the system employed. The selection of appropriate conditions, however, is well within the skill in the art.

In some embodiments, antigens are conjugated to biotin, which then can be removed with avidin or streptavidin bound to a support. In other embodiments, antigens are conjugated to fluorochromes, which can be used with a fluorescence activated cell sorter, to enable cell separation.

### H. AFFINITY MATURATION

The methods of the invention involving the cell surface display of proteins of interest are particularly useful for affinity maturation. According to the invention, a large number of substitutional variants can be generated, displayed on the cell surface of a plurality of cells, and selected for the desired target-binding characteristics by contacting the cells with target protein, Affinity maturation generally involves preparing and screening polypeptide variants, e.g., antibody variants, that have substitutions within the CDRs of a parent polypeptide and selecting variants that have improved biological properties such as binding affinity relative to the parent polypeptide. A convenient way for generating such substitutional variants is affinity maturation. Briefly, in some methods several hypervariable region sites (e.g. 6-7 sites) are mutated to generate amino substitutions at each site. The antibody variants thus generated are displayed in a monovalent fashion on the surface of a cell. The cell surface-displayed variants are then screened for their biological activity (e.g. binding affinity). See e.g., WO 92/01047, WO 93/112366, WO 95/15388 and WO 93/19172 for examples of phage display methods of affinity maturation.

Current antibody affinity maturation methods belong to two mutagenesis categories: stochastic and nonstochastic. Error prone PCR, mutator bacterial strains (Low et al., J. Mol. Biol. 260, 359-68 (1996)), and saturation mutagenesis (Nishimiya et al., J. Biol. Chem. 275:12813-20 (2000); Chowdhury, P. S. Methods Mol. Biol. 178, 269-85 (2002)) are typical examples of stochastic mutagenesis methods (Rajpal et al., Proc Natl Acad Sci USA. 102:8466-71 (2005)). Nonstochastic techniques often use alanine-scanning or site-directed mutagenesis to generate limited collections of specific variants. Some methods are described in further detail below.

1. Affinity Maturation Via Panning Methods

Affinity maturation of recombinant antibodies is commonly performed through several rounds of panning of candidate antibodies in the presence of decreasing amounts of antigen. Decreasing the amount of antigen per round selects the antibodies with the highest affinity to the antigen thereby yielding antibodies of high affinity from a large pool of starting material. Affinity maturation via panning is well known in the art and is described, for example, in Huls et al. (Cancer Immunol Immunother. 50:163-71 (2001)). The general concept is readily adaptable to the methods and materials of the present invention.

2. Look-Through Mutagenesis

Look-through mutagenesis (LTM) (Rajpal et al., Proc Natl Acad Sci USA. 102:8466-71 (2005)) provides a method for rapidly mapping the antibody-binding site. For L™, nine amino acids, representative of the major side-chain chemistries provided by the 20 natural amino acids, are selected to dissect the functional side-chain contributions to binding at every position in all six CDRs of an antibody. LTM generates a positional series of single mutations within a CDR where each “wild type” residue is systematically substituted by one of nine selected amino acids. Mutated CDRs are combined to generate combinatorial single-chain variable fragment (scFv) libraries of increasing complexity and size without becoming prohibitive to the quantitative display of all variants. After positive selection, clones with improved binding are sequenced, and beneficial mutations are mapped. Similarly, this general concept is readily adaptable to the methods and materials of the present invention.

3. Error-Prone PCR

Error-prone PCR involves the randomization of nucleic acids between different selection rounds. The randomization occurs at a low rate by the intrinsic error rate of the polymerase used but can be enhanced by error-prone PCR (Zaccolo et al., J. Mol. Biol. 285:775-783 (1999)) using a polymerase having a high intrinsic error rate during transcription (Hawkins et al., J Mol Biol. 226:889-96 (1992)). After the mutation cycles, clones with improved affinity for the antigen are selected using methods and materials of the present invention.

4. Gene Site Saturation Mutagenesis (GSSM)

GSSM involves the introduction of all possible base triplets at a given codon position, thereby resulting in the formation of a library containing all 20 amino acid exchanges at the target position. (Kretz et al, Meth. Enz. 388: 3-11 (2004)). This is achieved at the genetic level by using degenerate mutagenesis primers. Subsequent use of in vitro PCR amplification generates a library of genes possessing all codon variations required for complete saturation of the original gene.

5. Targeted Affinity Maturation™ (TAE)

TAE involves the use of degenerate codons that encode for an equal representation of eighteen amino acid residues including a stop codon and excluding cysteine and methionine. The degenerate codons each collectively code for eighteen amino acid residues eliminating any redundancy which may result in an over-representation of one or more amino acid residues. As a result, the method allows for the generation of smaller, focused libraries that contain eighteen amino acid substitutions at a position of interest (WO09/088,933). This general concept is readily adaptable to the methods and materials of the present invention.

6. DNA Shuffling

Nucleic acid shuffling is a method for in vitro or in vivo homologous recombination of pools of shorter or smaller polynucleotides to produce variant polynucleotides. DNA shuffling has been described in U.S. Pat. No. 6,605,449, U.S. Pat. No. 6,489,145, WO 02/092780 and Stemmer, Proc. Natl. Acad. Sci. USA, 91:10747-51 (1994). Generally, DNA shuffling is comprised of 3 steps: (1) fragmentation of the genes to be shuffled with DNase I, (2) random hybridization of fragments and reassembly or filling in of the fragmented gene by PCR in the presence of DNA polymerase (sexual PCR), and (3) amplification of reassembled product by conventional PCR.

DNA shuffling differs from error-prone PCR in that it is an inverse chain reaction. In error-prone PCR, the number of polymerase start sites and the number of molecules grows exponentially. In contrast, in nucleic acid reassembly or shuffling of random polynucleotides the number of start sites and the number (but not size) of the random polynucleotides decreases over time.

In the case of an antibody, DNA shuffling allows the free combinatorial association of all of the CDR1s with all of the CDR2s with all of the CDR3s, for example. It is contemplated that multiple families of sequences can be shuffled in the same reaction. Further, shuffling generally conserves the relative order, such that, for example, CDR1 will not be found in the position of CDR2. Rare shufflants will contain a large number of the best (e.g. highest affinity) CDRs and these rare shufflants may be selected based on their superior affinity.

The template polynucleotide which may be used in DNA shuffling may be DNA or RNA. It may be of various lengths depending on the size of the gene or shorter or smaller polynucleotide to be recombined or reassembled. Preferably, the template polynucleotide is from 50 bp to 50 kb. The template polynucleotide often should be double-stranded.

It is contemplated that single-stranded or double-stranded nucleic acid polynucleotides having regions of identity to the template polynucleotide and regions of heterology to the template polynucleotide may be added to the template polynucleotide, during the initial step of gene selection. It is also contemplated that two different but related polynucleotide templates can be mixed during the initial step. These techniques are readily adaptable to the methods and materials of the present invention.

### I. ALTERED GLYCOSYLATION

Antibody variants that are useful according to the present invention include antibodies that have a modified glycosylation pattern relative to the parent antibody, for example, deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. The presence of either of these tripeptide sequences in a polypeptide creates a potential glycosylation site. Thus, N-linked glycosylation sites may be added to an antibody by altering the amino acid sequence such that it contains one or more of these tripeptide sequences. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. O-linked glycosylation sites may be added to an antibody by inserting or substituting one or more serine or threonine residues to the sequence of the original antibody.

Also contemplated according to the invention are antibody molecules with absent or reduced fucosylation that exhibit improved ADCC activity. A variety of ways are known in the art to accomplish this. For example, ADCC effector activity is mediated by binding of the antibody molecule to the FcγRIII receptor, which has been shown to be dependent on the carbohydrate structure of the N-linked glycosylation at the Asn-297 of the CH2 domain. Non-fucosylated antibodies bind this receptor with increased affinity and trigger FcγRIII-mediated effector functions more efficiently than native, fucosylated antibodies. For example, recombinant production of non-fucosylated antibody in CHO cells in which the alpha-1,6-fucosyl transferase enzyme has been knocked out results in antibody with 100-fold increased ADCC activity (Yamane-Ohnuki et al., Biotechnol Bioeng. 87:614-22 (2004)). Similar effects can be accomplished through decreasing the activity of this or other enzymes in the fucosylation pathway, e.g., through siRNA or antisense RNA treatment, engineering cell lines to knockout the enzyme(s), or culturing with selective glycosylation inhibitors (Rothman et al., Mol. Immunol. 26:1113-23 (1989)). Some host cell strains, e.g. Lec13 or rat hybridoma YB2/0 cell line naturally produce antibodies with lower fucosylation levels. (Shields et al., J Biol. Chem. 277:26733-40 (2002); Shinkawa et al., J Biol. Chem. 278:3466-73 (2003)). An increase in the level of bisected carbohydrate, e.g. through recombinantly producing antibody in cells that overexpress GnTIII enzyme, has also been determined to increase ADCC activity (Umana et al., Nat. Biotechnol. 17:176-80 (1999)). It has been predicted that the absence of only one of the two fucose residues may be sufficient to increase ADCC activity (Ferrara et al., Biotechnol Bioeng. 93:851-61 (2006)).

### J. LABELS

In some embodiments, the cells and/or polypeptide binding agents are labeled to facilitate their detection. A “label” or a “detectable moiety” is a composition detectable by spectroscopic, photochemical, biochemical, immunochemical, chemical, or other physical means. For example, labels suitable for use in the present invention include, radioactive labels (e.g., ³²P), fluorophores (e.g., fluorescein), electron-dense reagents, enzymes (e.g., as commonly used in an ELISA), biotin, digoxigenin, or haptens as well as proteins which can be made detectable, e.g., by incorporating a radiolabel into the hapten or peptide, or used to detect antibodies specifically reactive with the hapten or peptide.

Examples of labels suitable for use in the present invention include, but are not limited to, fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold, colored glass or plastic beads (e.g., polystyrene, polypropylene, latex, etc.).

The label may be coupled directly or indirectly to the desired component of the assay according to methods well known in the art. Preferably, the label in one embodiment is covalently bound to the biopolymer using an isocyanate reagent for conjugation of an active agent according to the invention. In one aspect of the invention, the bifunctional isocyanate reagents of the invention can be used to conjugate a label to a biopolymer to form a label biopolymer conjugate without an active agent attached thereto. The label biopolymer conjugate may be used as an intermediate for the synthesis of a labeled conjugate according to the invention or may be used to detect the biopolymer conjugate. As indicated above, a wide variety of labels can be used, with the choice of label depending on sensitivity required, ease of conjugation with the desired component of the assay, stability requirements, available instrumentation, and disposal provisions. Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound.

The polypeptide binding agents useful according to the present invention can also be conjugated directly to signal-generating compounds, e.g., by conjugation with an enzyme or fluorophore. Enzymes suitable for use as labels include, but are not limited to, hydrolases, particularly phosphatases, esterases and glycosidases, or oxidotases, particularly peroxidases. Fluorescent compounds, i.e., fluorophores, suitable for use as labels include, but are not limited to, fluorescein and its derivatives, rhodamine and its derivatives, dansyl, umbelliferone, etc. Further examples of suitable fluorophores include, but are not limited to, eosin, TRITC-amine, quinine, fluorescein W, acridine yellow, lissamine rhodamine, B sulfonyl chloride erythroscein, ruthenium (tris, bipyridinium), Texas Red, nicotinamide adenine dinucleotide, flavin adenine dinucleotide, etc. Chemiluminescent compounds suitable for use as labels include, but are not limited to, luciferin and 2,3-dihydrophthalazinediones, e.g., luminol. For a review of various labeling or signal producing systems that can be used in the methods of the present invention, see U.S. Pat. No. 4,391,904.

Means for detecting labels are well known to those of skill in the art. Thus, for example, where the label is radioactive, means for detection include a scintillation counter or photographic film, as in autoradiography. Where the label is a fluorescent label, it may be detected by exciting the fluorochrome with the appropriate wavelength of light and detecting the resulting fluorescence. The fluorescence may be detected visually, by the use of electronic detectors such as charge coupled devices (CCDs) or photomultipliers and the like. Similarly, enzymatic labels may be detected by providing the appropriate substrates for the enzyme and detecting the resulting reaction product. Colorimetric or chemiluminescent labels may be detected simply by observing the color associated with the label. Other labeling and detection systems suitable for use in the methods of the present invention will be readily apparent to those of skill in the art. Such labeled modulators and ligands can be used in the diagnosis of a disease or health condition.

Although the foregoing invention has been described in detail for purposes of clarity of understanding, it will be obvious that certain modifications may be practiced within the scope of the appended claims. All publications and patent documents cited in the present application are hereby incorporated by reference in their entirety for all purposes to the same extent as if each were so individually denoted. The following examples are provided for illustrative purposes and are not intended to limit the scope of the invention.

### EXAMPLES

### Example 1

### General Materials and Methods

### Enzymes, Antibodies and Recombinant Proteins

Restriction endonucleases and T4 DNA ligase were purchased from New England Biolabs (Ipswich, Mass.). PCR was performed using KOD Hot Start Polymerase (EMD4Biosciences, Gibbstown, N.J.). Antibodies for flow cytometry were obtained from Invitrogen Corp. (Carlsbad, Calif.). Recombinant human IL-1β was purchased from PeproTech (Rocky Hill, N.J.) and labeled with biotin using the EZ-Link Sulfo-NHS-biotin labeling kit (Thermo Scientific, Rockford, Ill.) according to the manufacturer's instructions.

### Yeast Clones and Strains

Yeast clones YNR044W (AGA1) and YGL032C (AGA2) were purchased from Open Biosystems (Huntsville, Ala.) while the Saccharomyces cerevisiae strain BJ5465 was obtained from ATCC. The Saccharomyces cerevisiae strain EBY100 is described in Boder and Wittrup, Nat. Biotech. 15, 553-557 (1997).

### Media and Buffers

SDCAA media: 38 mM Na₂HPO₄, 71 mM NaH₂PO₄, 2% (w/v) D-dextrose, 0.67% (w/v) yeast nitrogen base, 0.5% (w/v) casamino acids, pH 7.5.

SGCAA media: same as SDCAA with galactose instead of D-dextrose.

Both SDCAA and SGCAA media, tryptophan, uracil, and phosphate buffered saline (PBS) were purchased from TEKnova (Hollister, Calif.). Wash buffer for flow cytometry consists of filter sterilized PBS containing 0.1% (w/v) BSA (Sigma-Aldrich, St. Louis, Mo.).

Transfection media: HyClone SFMTransfx-293 Media supplemented with 4 mM L-glutamine.

Growth media: HyClone SFMTransfx-293 Media, 10% (w/v) HyClone FBS, 4 mM L-glutamine, and 250 geneticin.

Both HyClone SFMTransfx-293 media and FBS were purchased from Thermo Scientific (Rockford, Ill.) while L-gluatamine and geneticin were purchased from Invitrogen Corp (Carlsbad, Calif.)

### Yeast Transformation and Growth

Chemically competent yeast cells were prepared using Frozen-EZ Transformation II™ kit (Zymo Research, Orange, Calif.). Transformed cells were grown on dextrose media plates for 72 hours at 30° C. Isolated colonies were then grown as cultures for 16 hours at 30° C. with shaking (250 rpm). The dextrose media was replaced with galactose media and cells were grown for 20 hours to induce antibody expression.

### Mammalian Cell Transfection and Growth

Twenty micrograms of plasmid DNA was incubated with 20 μg of Lipofectamine™ 2000 reagent (Invitrogen Corp, Carlsbad, Calif.) in 1 ml of transfection media for 25 minutes at room temperature. The mixture was then added drop-wise to 1.6×10⁷ human embryonic kidney (HEK) 293E cells in 20 ml of transfection media. Cells were then grown at 37° C. with 5% CO₂ and shaking (95 rpm). Cells were harvested after 4 days for flow cytometric analysis and 7 days for protein expression and purification. To purify soluble IgG, transfected cells were centrifuged (3200 rpm for 10 minutes at 4° C.) and the conditioned media was removed and incubated with 200 μl of Protein A Sepharose CL-4B (GE Healthcare, Waukesha, Wis.) either for 2 hours at room temperature or for 16 hours at 4° C. The resin was washed with PBS and incubated with 700 μl of elution buffer (0.2 M Glycine/HCl, pH 2.5) followed by the addition of neutralization buffer (1 M Tris-HCl, pH 9.0). Purified IgGs were then dialyzed into PBS for 16 hours at 4° C. and analyzed by HPLC and SDS-PAGE for purity.

### Flow Cytometry

Two million yeast cells or 5×10⁴ HEK293 cells were washed with wash buffer then incubated with biotin labeled IL-1β (100 nM) and chicken anti-c-Myc (4 μg/ml) for 1 hour at room temperature. The cells were then washed and incubated with streptavidin-phycoerythrin (PE) (10 μg/ml), anti-chicken Alexa Fluor® 647 conjugate (20 μg/ml) and anti-hemagglutinin (HA) Alexa Fluor® 488 conjugate (10 μg/ml) for 30 minutes on ice and protected from light. The cells were then washed one final time before being analyzed on a either a C6 flow cytometer (Accuri Cytometers Inc., Ann Arbor, Mich.) or a FACScan instrument (BD, Franklin Lakes, N.J.). Typically, 10,000 events were collected per sample. Subsequent analysis was performed using FlowJo software (Tree Star Inc., Ashland, Oreg.).

### Example 2

### Construction of Aga2 Fusion Display Vector as a Benchmark

The vector pTam14 containing GAL1 promoter, DNA coding for Aga2 signal peptide (1-18), BamHI restriction site for cloning, DNA coding for c-Myc epitope tag, DNA coding for the mature Aga2 protein (19-87), MATα transcription terminator, TRP1 gene, CEN6/ARSH4 origin, AMP resistance gene, and pUC bacterial origin was synthesized by GenScript (Piscataway, N.J.).

The clone, XPA28 scFv, was identified after three rounds of soluble panning of an antibody phage display library against biotin labeled IL-1β. Sequencing revealed that XPA28 scFv possessed a heavy chain and lambda light chain corresponding to families VH3 and VL1. XPA28 scFv was subcloned into the vector pXHMV (Rondon et al. PCT Publication No. WO 2010/040073) in which the scFv is fused in frame with the epitope tags 6×His, c-Myc, V5, and pI11 from bacteriophage.

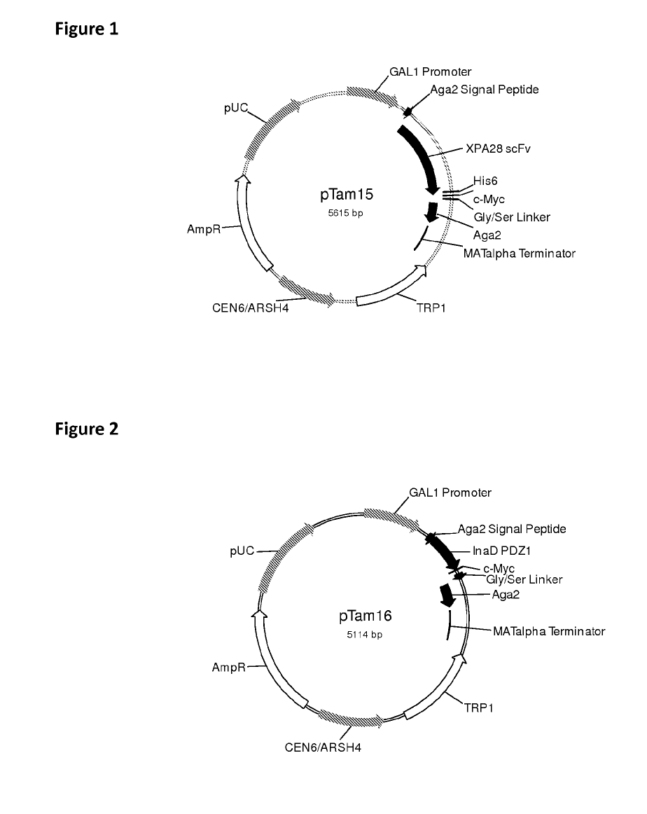

The vector pTam15 was constructed by first PCR amplifying XPA28 scFv from the plasmid pXHMV/XPA28 scFv using forward (Tam40b: TCTGTTATTGCTAGCGTTTTAGCACAGGTCCAGCTGGTGCAG) (SEQ ID NO: 18) and reverse (Tam41: CTTTTGTTCGGATCCTGCGGCCCCGTGATGGTG) (SEQ ID NO: 19) primers and digested using NheI and BamHI as was the acceptor vector pTam14. The fragment and vector were ligated resulting in the vector pTam15 (FIG. 1).

### Example 3

### Vector Construction for the NorpA Tether Display System

The vector pTam16 was constructed by first synthesizing the PDZ1 domain of InaD (11-107) and then PCR amplifying the fragment using the forward (Tam55: GTTATTGCTAGCGTTTTAGCAGCGGGTGAGCTC) (SEQ ID NO: 20) and reverse (Tam56: TTGTTCGGATCCCTTGTCGAAGGTCTGA) (SEQ ID NO: 21). Both the amplified fragment and pTam14 were digested NheI and BamHI and ligated together resulting in the vector pTam16 (FIG. 2).

The vector pTam21 was constructed as follows: DNA coding for the V5 tag (GKPIPNPLLGLDST) (SEQ ID NO: 22) in pXHMV/XPA28 scFv was replaced with DNA coding for the NorpA tether which consists of the C-terminal seven residues (GKTEFCA) (SEQ ID NO: 16) of NorpA (1089-1095). This was done by QuikChange™ site-directed mutagenesis (Stratagene, La Jolla, Calif.) using mutagenic primers Tam59 (GATCTGAAGGCCGCAGGCAAGACCGAGTTCTGCGCCTGATGAGAGGCTAGTTCT GC) (SEQ ID NO: 23) and Tam60 (GCAGAACTAGCCTCTCATCAGGCGCAGAACTCGGTCTTGCCTGCGGCCTTCAGA TC) (SEQ ID NO: 24). The DNA corresponding to XPA28 scFv including His6, c-myc tag and NorpA was PCR amplified using forward (Tam71: GTTATTGCTAGCGTTTTAGCACAGGTCCAGCTGGTG) (SEQ ID NO: 25) and reverse (Tam72: CAGCGGGTTTAAACTCATCAGGCGCAGA) (SEQ ID NO: 26) primers, digested with NheI and PmeI and ligated into NheI/PmeI digested pTam14.

The vector pTam27 was constructed by QuikChange™ mutagenesis of the plasmid pTam16 using mutagenic primers Tam93a (AATATTTTCTGTTATTGCCAGCGTTTTAGCAGCGGGTGAG) (SEQ ID NO: 27) and Tam94 (CTCACCCGCTGCTAAAACGCTGGCAATAACAGAAAATATT) (SEQ ID NO: 28). This introduced a silent mutation to abolish a NheI restriction site within the Aga2 signal peptide.

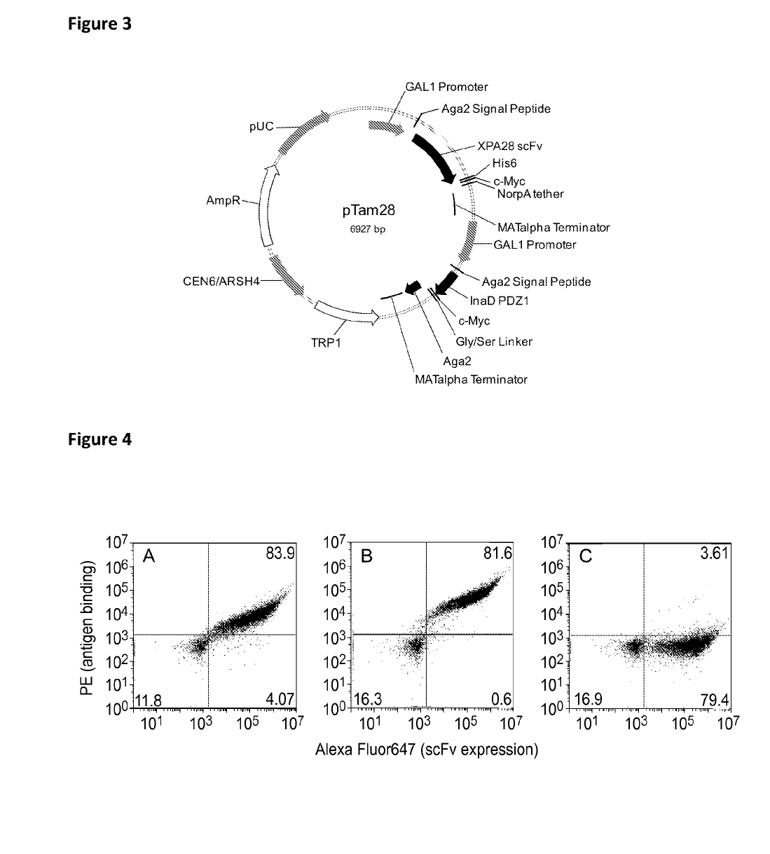

The vector pTam28 was constructed as follows: the DNA corresponding to the Aga2 signal peptide, XPA28 scFv, His6 tag, c-Myc tag, NorpA tether, and MATα terminator was PCR amplified using pTam21 as the template and forward (Tam89: GTTATTGCTAGCGTTTTAGC) (SEQ ID NO: 29) and reverse (Tam90: CGGCTTCTAATCCGTGTTATTACTGAGTAGTATTTATTTAAG) (SEQ ID NO: 30) primers. The DNA corresponding to the Gal1 promoter, Aga2 signal peptide, InaD PDZ1 and c-Myc tag were PCR amplified using pTam27 as template and forward (Tam91: CTACTCAGTAATAACACGGATTAGAAGCCG) (SEQ ID NO: 31) and reverse (Tam92: GGAGATAAGCTTTTGTTCG) (SEQ ID NO: 32) primers. The two PCR fragments contain 30 base pairs of homology and were combined using overlap PCR extension. Briefly, a mixture of the two fragments (100 ng each) was thermocycled for 6 cycles before pausing at the final extension step (70° C.). Subsequently the outermost primers (Tam89 and Tam92) were added to a final concentration of 300 nM and the reaction was allowed to continue for an additional 26 cycles. A DNA band corresponding to the combined fragment (2127 base pairs) was gel purified and digested using NheI and HindIII, as was the acceptor vector pTam14. The fragment and vector were subsequently ligated resulting in the vector pTam28 (FIG. 3).

### Example 4

### Construction and Validation of the NorpA Tether Display System

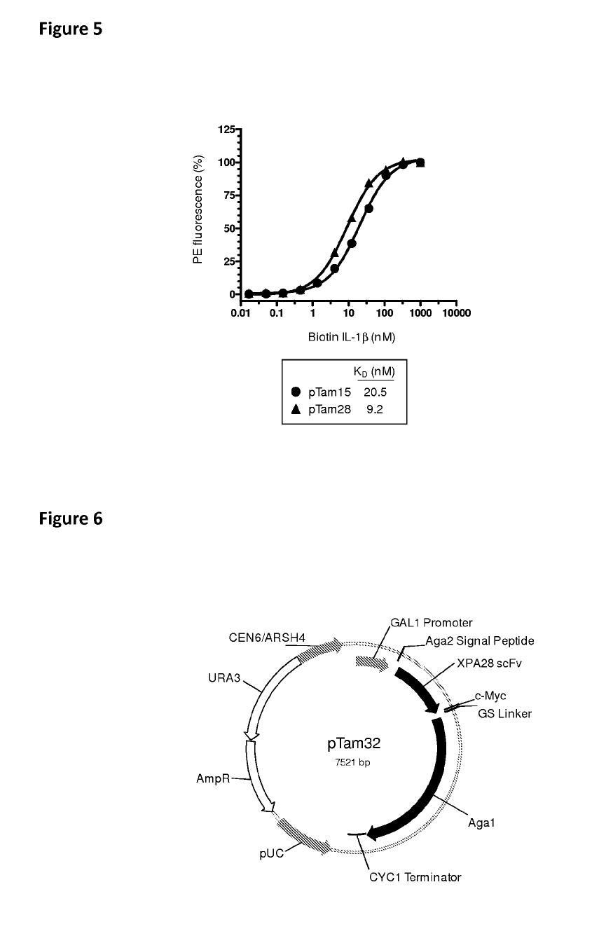

EBY100 cells were transformed with pTam15, 16 and 28 and grown on SDCAA media. Single colonies were grown and induced with SGCAA media. For flow cytometry analysis, cells were washed and labeled as described in Example 1. As shown in FIG. 4A, cells displaying XPA28 scFv using the NorpA tether system (pTam28) bind IL-10 and are positive for c-Myc staining as observed by PE and Alexa Fluor 647 fluorescence. However, antigen binding and scFv expression is not a direct correlation as the c-Myc epitope tag is present at both the carboxyl terminus of XPA28 scFv and the InaD PDZ1. Compared to cells displaying XPA28 scFv as an Aga2 fusion (pTam15, FIG. 4B), the amount of IL-1β binding by the NorpA tether system is lower. As a control, cells expressing InaD PDZ1 alone (pTam16) do not bind IL-1β (FIG. 4C). Cells expressing pTam15 and 28 were then incubated with increasing concentrations of biotin labeled IL-10 (16.9 pM-1 μM) and analyzed by flow cytometry. The mean PE fluorescence for each sample was determined and calculated as a percentage of total. This was then plotted against IL-10 concentration and the dissociation constant (K_{D}) was determined as the concentration at the half maximum (EC₅₀). As shown FIG. 5, the K_{D} for IL-1β binding by XPA28 scFv using the two display systems was similar: 9.2 nM for the NorpA tether system (pTam28) and 20.5 nM for Aga2 fusion display (pTam15); and within the range of error for this type of analysis (Chao, Lau et al. 2006). These results suggest that the affinity of XPA28 for IL-1β is not compromised in the NorpA tether system. Rather the lower level of antigen binding observed using the NorpA tether system (FIG. 4A) compared to the Aga2 fusion display (FIG. 4B) may be a result of a decrease in the total level of scFv found on the cell surface. Finally, the K_{D} determined using NorpA tether system is in good agreement to the K_{D} for soluble XPA28 reformatted as an IgG1 (15.1±4.5 nM) as determined by Biacore.

The vector pTam18 was constructed as follows: a DNA fragment consisting of DNA coding for Aga2 signal peptide (1-18), XPA28 scFv, c-Myc tag, glycine serine linker and Aga2 (19-87) was generated by overlap extension PCR using four smaller fragments. First, an oligo corresponding to Aga2 signal peptide was synthesized (RbsAga2SS 1: CGACTCACTATAGGGAATATTAAGCTAATTCTACTTCATACATTTTCAATTAAGA TGCAGTTACTTCGCTGTTTTTCAATATTTTCTGTTATTGCTAGCGTTTTAGCACAG GTCCAGCTGGTG) (SEQ ID NO: 33) and PCR amplified using forward and reverse primers (RbsAga2SS 2: CGACTCACTATAGGGAATATTAAG (SEQ ID NO: 34) and RbsAga2SS 3: CACCAGCTGGACCTG (SEQ ID NO: 35), respectively). The vector pXHMV/XPA28 scFv was used as the template for PCR amplification using the following forward (IL1 scFv 1: CAGGTCCAGCTGGTGCAG (SEQ ID NO: 36) and reverse (IL1 scFv 2: TGCGGCCCCGTG (SEQ ID NO: 37)) primers. An oligo corresponding to c-Myc epitope tag and glycine serine linker was synthesized (MycGS 1: CACGGGGCCGCAGGATCCGAACAAAAGCTTATCTCCGAAGAAGACTTGGGTGGT GGTGGATCTGGTGGTGGTGGTTCTGGTGGTGGTGGTTCTCAGGAACTGACAACTA TATGCGAG) (SEQ ID NO: 38) and PCR amplified using forward and reverse primers (MycGS 2: CACGGGGCCGCA (SEQ ID NO: 39) and MycGS 3: CTCGCATATAGTTGTCAGTTCCTG (SEQ ID NO: 40), respectively). The mature Aga2 protein (19-87) was amplified from YGL032C (Open Biosystems, Huntsville, Ala.) using the forward (Aga2 1: CAGGAACTGACAACTATATGCGAG) (SEQ ID NO: 41) and reverse (Aga2 2: GGATCAGCGGGTTTAAACTCAAAAAACATACTGTGTGTTTATGGG) (SEQ ID NO: 42) primers. The four fragments were gel purified separately and combined by overlap extension PCR as described in Example 1 using primers RbsAga2SS 2 and Aga2 2. A single fragment (1209 base pairs) corresponding to the size of all four fragments was gel purified and cloned into SacI/PmeI digested pYC2/CT (Invitrogen Corp, Carlsbad, Calif.) using In-Fusion® cloning (Clontech, Mountain View, Calif.).

The vector pTam32 was constructed as follows: a DNA fragment coding for c-Myc tag, glycine serine linker and mature Aga1 protein (23-726) was generated by overlap extension PCR using two fragments. An oligo corresponding to c-Myc tag and a glycine serine linker was synthesized (Tam121: GAACAAAAGCTTATCTCCGAAGAAGACTTGGGTGGTGGTGGATCTGGTGGTGGT GGTTCTGGTGGTGGTGGTTCTTTGGCATCTGATCC) (SEQ ID NO: 43) and PCR amplified using forward (Tam159: GAACAAAAGCTTATCTCCG) (SEQ ID NO: 44) and reverse (Tam131: GGATCAGATGCCAAAGA) (SEQ ID NO: 45) primers. Aga1p (23-176) was amplified from clone YNR044W (Open Biosystems, Huntsville, Ala.) using forward (Tam133: TTTGGCATCTGATCC) (SEQ ID NO: 46) and reverse (Tam122: CAGCGGGTTTAAACTTAACTGAAAATTACATTGC) (SEQ ID NO: 47) primers. The two fragments were gel purified and combined by overlap extension PCR using primers Tam159 and Tam122 to amplify. The combined fragment and pTam18 were both digested with HindIII and PmeI and ligated, resulting in the vector pTam32 (FIG. 6).

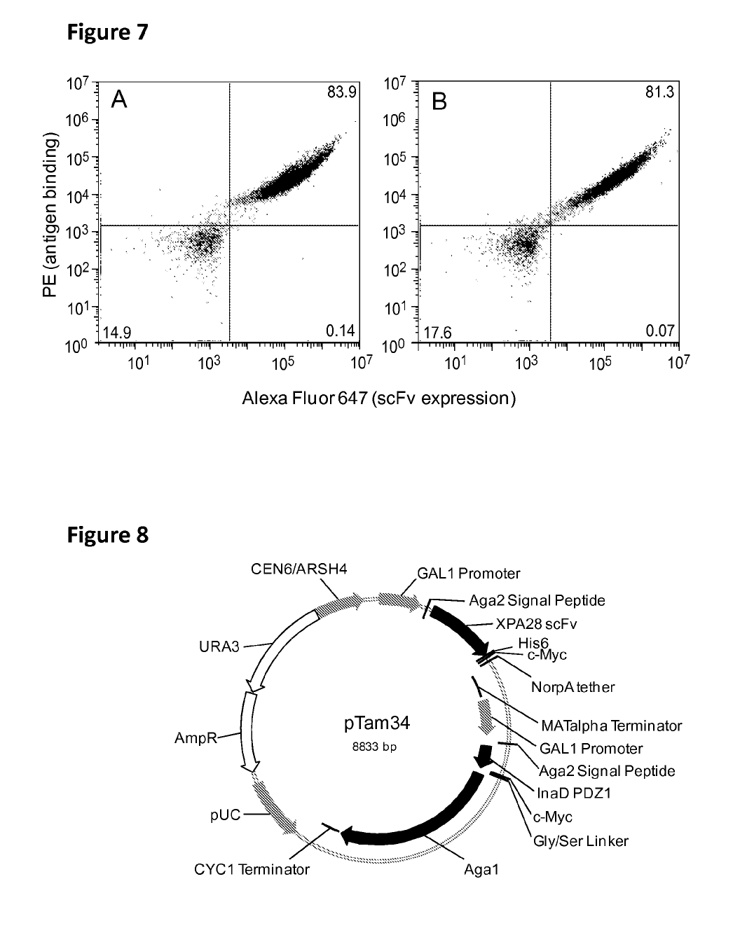

EBY100 and BJ5465 cells were transformed with pTam15 and 32 respectively. Transformants were identified after growth on either SDCAA (EBY100, pTam15) or SDCAA containing 40 μg/ml tryptophan (BJ5465, pTam32). Single colonies were then grown in liquid media and induced with the corresponding galactose media (SGCAA or SGCAA with 40 μg/ml tryptophan). For flow cytometry analysis, cells were washed, labeled and analyzed as described in Example 1. As shown in FIG. 7, the antigen binding and c-Myc staining properties of pTam15 (A) and pTam32 (B) transformed cells were very similar as observed by PE and Alexa Fluor 647 fluorescence, indicating that Aga1 is a suitable alternative to Aga2 as a cell wall anchor for XPA28 scFv.

pTam34 was constructed by first eliminating the NheI restriction site between the URA3 gene and CEN6/ARSH4 origin of replication in pTam32 using QuikChange™ mutagenesis and the following forward (Tam137: GATGAATTGAATTGAAAAGCTAGTTTATCGATGGGTCCTTTTCATCACGTGC) (SEQ ID NO: 48) and reverse (Tam138: GCACGTGATGAAAAGGACCCATCGATAAACTAGCTTTTCAATTCAATTCATC) (SEQ ID NO: 49) primers. The resulting plasmid, pTam32b was digested with NheI and HindIII and served as the acceptor vector for the following insert. A DNA fragment corresponding to XPA28 scFv, His6, c-Myc tag, NorpA tether, MATα terminator, GAL1 promoter, InaD PDZ1, and c-Myc tag was excised from pTam28 using NheI and HindIII. The insert and the acceptor vector were ligated resulting in the vector pTam34 (FIG. 8).

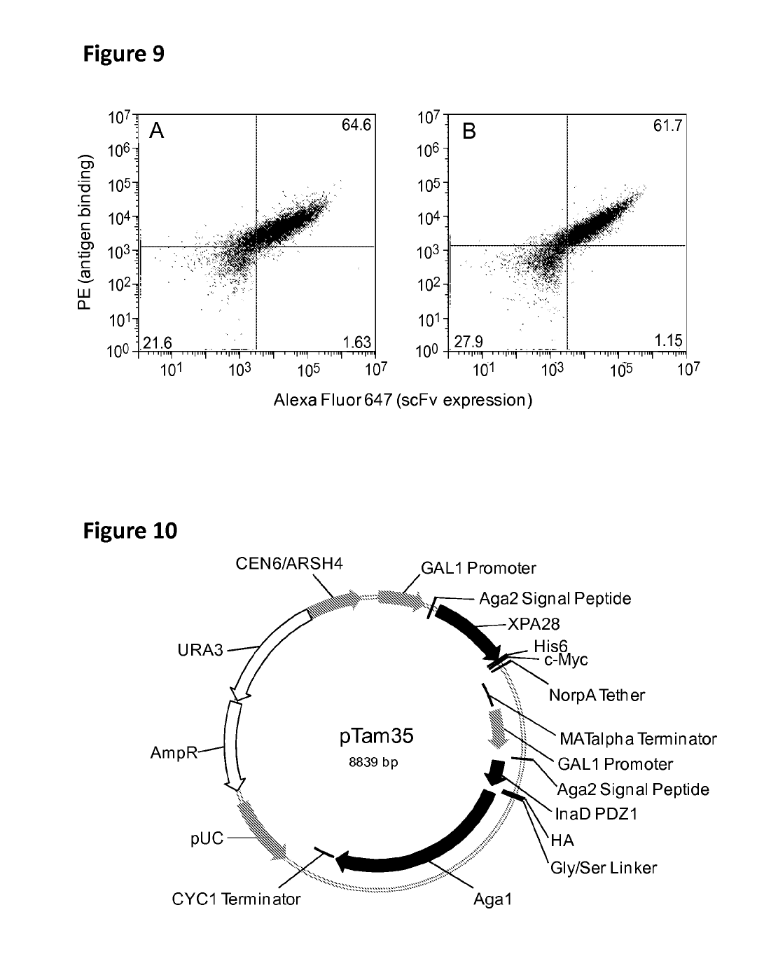

EBY100 and BJ5465 cells were transformed with pTam28 and 34 respectively and grown on either SDCAA (EBY100, pTam28) or SDCAA containing 40 μg/ml tryptophan (BJ5465, pTam34). Cells were induced with the corresponding galactose media (SGCAA or SGCAA with 40 μg/ml tryptophan) prior to labeling for flow cytometry analysis. As shown in FIG. 9, the antigen binding and c-Myc staining properties of pTam28 (A) and pTam34 (B) transformed cells were very similar as observed by PE and Alexa Fluor 647 fluorescence. This indicates that Aga1 was a suitable alternative to Aga2 in anchoring the InaD PDZ1 to the yeast cell wall.

pTam35 was constructed as follows: the DNA sequence (Tam68: ACCTTCGACAAGAGATCCTGTTACCCATACGACGTTCCAGACTACGCTTCTTTGG GTGGTGGTGGATCTGGT) (SEQ ID NO: 50) corresponding to the hemagglutinin (HA) epitope (CYPYDVPDYASL) (SEQ ID NO: 51) was synthesized and PCR amplified using forward (Tam69: ACCTTCGACAAGAGATCC) (SEQ ID NO: 52) and reverse (Tam70: ACCAGATCCACCACC) (SEQ ID NO: 53) primers. The amplified insert contains regions of homology to the sequence immediately 5′ and 3′ to the c-Myc tag at the carboxyl terminus of InaD PDZ1. The plasmid pTam34 was digested with HindIII and the insert was cloned using In-Fusion® cloning. As shown in the vector map for pTam35, the c-Myc epitope tag fused to InaD PDZ1 is now replaced with a HA tag (FIG. 10).

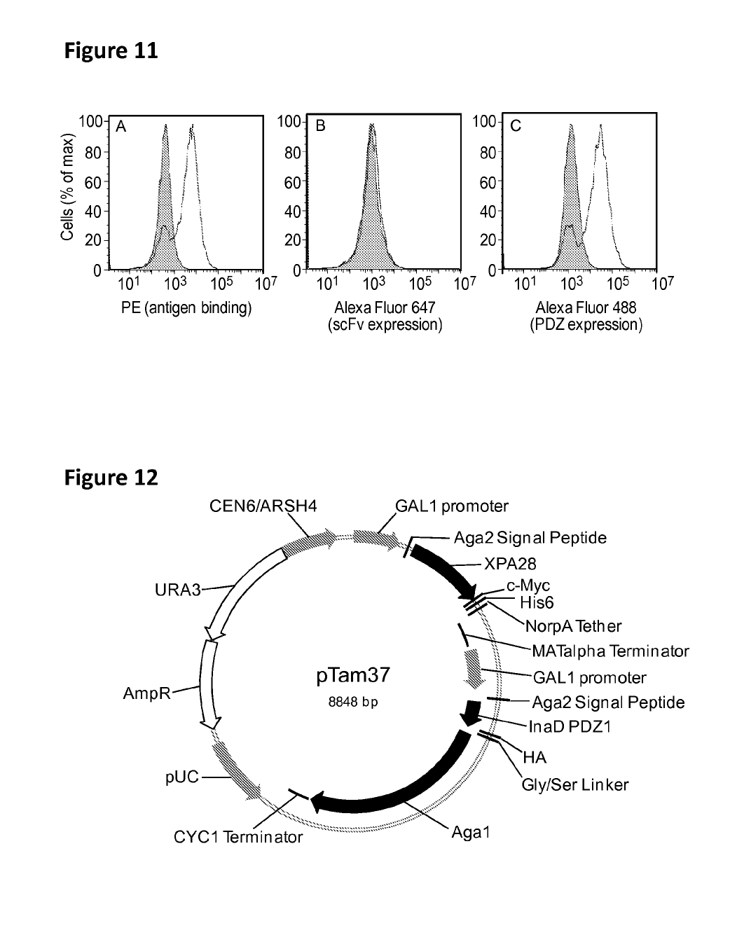

BJ5465 cells were transformed with pTam35, grown with SDCAA containing 40 μg/ml tryptophan and induced with SGCAA containing 40 μg/ml tryptophan prior to labeling. Both non-induced and induced cells were analyzed by flow cytometry (FIG. 11). As shown in FIG. 11, induced cells (no fill) bind IL-1β (A) and express InaD PDZ1 (C) as assessed by PE and Alexa Fluor 488 fluorescence, compared to non-induced cells (grey fill). However no scFv was detected as observed by Alexa Fluor 647 fluorescence (B). As the cells did bind IL-1β, this suggests that the lack of Alexa Fluor 647 fluorescence was due to the inability of the c-Myc antibody to bind the epitope tag and not due to the lack of scFv expression. We further postulated that the complex formation between the NorpA tether and InaD PDZ1 may occlude the binding of the c-Myc antibody to the tag. To address this issue, we constructed the vector pTam37, in which the c-Myc epitope tag was separated further in primary sequence from the NorpA tether.

The vector pTam37 was constructed by first PCR amplifying the vector backbone of pTam35 using forward (Tam145: CCGCTGATCTGATAACAA) (SEQ ID NO: 54) and reverse (Tam146: GTCAGCTTGGTCCCA) (SEQ ID NO: 55) primers. The DNA fragment (Tam142: TGGGACCAAGCTGACCGTCCTAGGCCTCGGGGGCCTGGAACAAAAACTCATCTC AGAAGAAGATCTGGGAGGGGCCGCACATCATCATCACCATCACGGTGGCGCCGC CGGCAAGACCGAGTTCTGCGCCTGATGAGTTTAAACCCGCTGATCTGATAACAA) (SEQ ID NO: 56) which corresponds to c-Myc, His6 and NorpA tether in this specific order was synthesized and PCR amplified with forward (Tam143: TGGGACCAAGCTGAC) (SEQ ID NO: 57) and reverse (Tam144: TTGTTATCAGATCAGCGG) (SEQ ID NO: 58) primers. The amplified fragment contains 18 base pairs of homology to the amplified backbone which allowed for the cloning using In-Fusion®. As shown in FIG. 12, pTam37 differs from pTam35 in that the c-Myc tag now precedes the His6 tag.

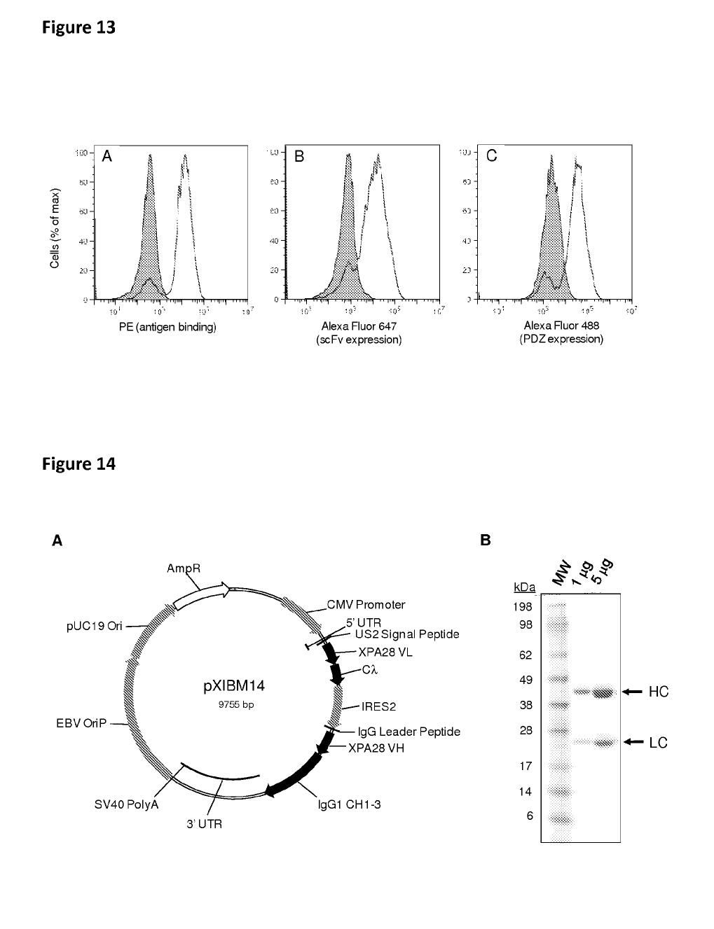

BJ5465 cells were transformed with pTam37, grown with SDCAA containing 40 μg/ml tryptophan and induced with SGCAA containing 40 μg/ml tryptophan prior to labeling. Both non-induced and induced cells were analyzed by flow cytometry. As shown in FIG. 13, induced cells (no fill) bind IL-1β (A) and express InaD PDZ1 (B) whereas non-induced cells (grey fill) do not. A comparison of Alexa Fluor 647 fluorescence between pTam37 (FIG. 13B) and pTam35 (FIG. 11B) indicates a significant difference in detection of the c-Myc epitope tag presumably due to a difference in tag accessibility between the two vectors. In light of these results, the plasmid pTam37 was chosen as the vector for construction of a small non-immune scFv library.

### Example 5

### NorpA Tether Display of an IgG on Mammalian Cells

An IgG1 antibody, anti-KLH8, was used to as a model antibody for evaluating protein expression during the vector construction process. The DNA corresponding to the entire heavy chain (HC) of anti-KLH8 was PCR amplified using the following primers: KLH8-HC-XbaI Fwd. Primer (ATATATTCTAGAATGGGATGGTCATGTATCATC) (SEQ ID NO: 59) and KLH8-HC-NotI Rev. Primer (ATATATGCGGCCGCTCATTTACCCGGGGACAGGGA) (SEQ ID NO: 60) and ligated into the pIRES vector (Clontech, Mountain View, Calif.) by restriction site cloning using XbaI and NotI. This plasmid was then digested with EcoRI and XbaI and a synthetic IRES2 sequence (Blue Heron, Bothell, Wash.) was cloned in place of IRES 1. The IRES2 sequence and the anti-KLH8 HC were then PCR amplified using primers: IRES2 Xho1 Fwd. Primer (ATATATCTCGAGAATTCACGCGTCGAGCATGCAT) (SEQ ID NO: 61) and Xho1-KLH8 HC Rev. Primer (ATATATCTCGAGTCATTTACCCGGGGACAGGGA) (SEQ ID NO: 62). The amplified fragment was digested with XhoI and cloned into the acceptor vector, pMXT13/anti-KLH8 VL, a transient expression vector containing the constant lambda (CX) light chain (XOMA, Berkeley, Calif.; WO06/060769) and the anti-KLH8 VL at the single XhoI site. The resulting vector pXIBM-IRES2, in which the anti-KLH8 LC and HC are regulated by a CMV promoter and IRES2 respectively, now allows the expression of a full IgG from a single vector. Next, the IgG leader peptide preceding the anti-KLH8 LC was replaced with US2 signal peptide (U.S. Pat. No. 7,094,579). A DNA fragment consisting of the US2 signal peptide, anti-KLH8 LC, and IRES2 was generated by a two step PCR reaction. The primary PCR reaction consisted of amplifying the anti-KLH8 LC and IRES2 using an extended forward primer containing the US2 sequence (US2-KLH8 LC Fwd. Primer: TTCTGCTTGTGGCCCTGCAGGCCCAAGCGCAGCCTGTGCTGACTCAGCCC) (SEQ ID NO: 63) and a reverse primer (IRES2-PmlI Rev. Primer: GCAGGTGTATCTTATACACGTGGC) (SEQ ID NO: 64). A secondary PCR reaction using the reverse primer above and a second forward primer (Sal1-US2 Fwd. Primer: ATATATGTCGACACCATGCGTACTCTGGCTATCCTTGCAGCTATTCTGCTTGTGGC CCTGCAGGCCCAA) (SEQ ID NO: 65) was done to complete the US2 signal sequence and to add a SalI site for cloning. The PCR fragment was then cloned into pXIBM-IRES2 by restriction site cloning using the SalI and PmlI sites resulting in the vector pXIBM-US2-IRES2. Finally, the anti-KLH8 LC and HC were replaced with the corresponding variable regions of XPA28. The XPA28 LC and HC were amplified from the template pXHMV/XPA28 scFv using the following primers: Sfi1-VL Fwd. primer (ATATATGTGGCCCTGCAGGCCCAAGCGCAGGCTGTGCTGACTCAGCCG) (SEQ ID NO: 66), VL AvrII Rev. primer (ATATATAGGCCTAGGACGGTCAGCTTGGT) (SEQ ID NO: 67), Nco1-VH Fwd. primer (ATATATGCCATGGCCCAGGTCCAGCTGGTGCAGTCT) (SEQ ID NO: 68), and VH Nhe1 Rev. primer (ATATATGCTAGCACTGGAGACGGTGACCAGGGTGCCT) (SEQ ID NO: 69). The LC and HC of XPA28 were inserted into pXIBM-US2-IRES2 by restriction site cloning using SfiI/AvrII and NcoI/NheI restriction enzyme pairs respectively. The resulting vector pXIBM14 (FIG. 14A) features the VL and VH of XPA28 fused in frame with Cλ and the CH1-3 regions of IgG1 respectively. Cells transiently transfected with pXIBM14 expressed soluble XPA28 IgG which was purified by Protein A Sepharose. The electrophoretic mobility of the HC and LC were as expected, indicating that the XPA28 scFv was correctly reformatted as a full length antibody (XPA28 IgG) (FIG. 14B).

In order to display the XPA28 IgG on the cell surface, the NorpA tether was fused to the carboxyl terminus of the heavy chain. The NorpA tether was PCR amplified from pTam37 using the forward primer Nhe1-HC Fwd (ATATATGCTAGCACAAAGGGCCCATCGGTCTTC) (SEQ ID NO: 70) and one of the following three reverse primers in order to generate a tether with no attached spacer (Xho1-PDZ-NoAA Rev: ATATATCTCGAGTCAGGCGCAGAACTCGGTCTTGCCTTTACCCGGGGACAGGGAG AG) (SEQ ID NO: 71), a 3 amino acid spacer (Xho1-PDZ-3AA Rev: ATATATCTCGAGTCAGGCGCAGAACTCGGTCTTGCCTGCGGCCCCTTTACCCGGG GACAGGGAGAG) (SEQ ID NO: 72), and a 5 amino acid attached spacer (Xho1-PDZ-5AA Rev: ATATATCTCGAGTCAGGCGCAGAACTCGGTCTTGCCTGAACCGCCGCCTCCTTTA CCCGGGGACAGGGAGAG) (SEQ ID NO: 73).

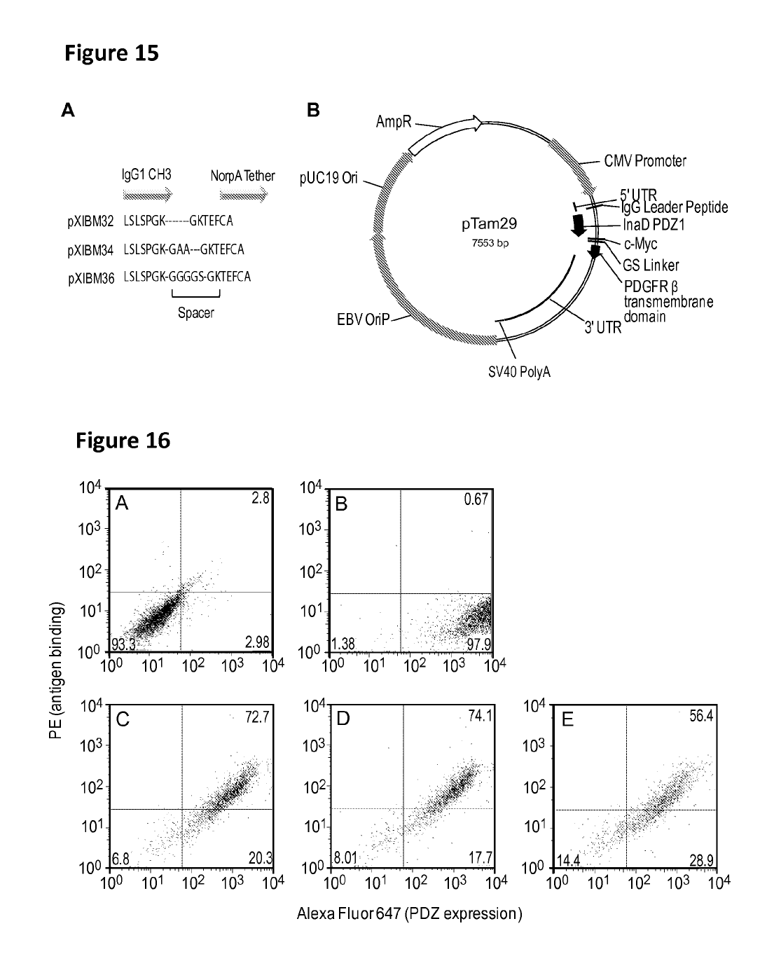

The three different NorpA tether PCR fragments were cloned into pXIBM14 at the Nhe1 and Xho1 restriction sites generating the three resulting vectors: pXIBM32, which contains no spacer between the CH3 and NorpA tether; pXIBM34, which contains the 3 amino acid spacer (GAA) spacer; and pXIBM36, which contains the five amino acid spacer (GGGGS) (SEQ ID NO: 74) are shown in FIG. 15.

The vector pTam29 was constructed as follows: a DNA fragment coding for the IgG leader peptide, InaD PDZ1, c-Myc epitope, glycine serine linker and the transmembrane domain of platelet derived growth factor receptor-β (PDGFR-β) was generated by overlap extension PCR using two fragments. The DNA corresponding to InaD PDZ1 was PCR amplified using forward (Tam95: AACTGCAACTGGAGTGCATTCCGCGGGTGAGCTCATTCACAT) (SEQ ID NO: 75) and reverse (Tam96: CTGGCCCACAGCAGAACCACCACCACCAGAACC) (SEQ ID NO: 76) primers. The 49 amino acid transmembrane domain of PDGFR-β (513-561) was PCR amplified from the plasmid pDisplay (Invitrogen, Carlsbad, Calif.) using forward (Tam97: GGTTCTGGTGGTGGTGGTTCTGCTGTGGGCCAG) (SEQ ID NO: 77) and reverse (Tam98: CTTTGTGACGGGCGGGCTCGAGGCCGTCGCACCTAACGTGGCTTCTTC) (SEQ ID NO: 78) primers. The two fragments were gel purified and combined by overlap extension PCR using primers Tam99 (AACTGCAACTGGAGTGCATTCC) (SEQ ID NO: 79) and Tam100 (CTTTGTGACGGGCGGG) (SEQ ID NO: 80). Both the combined fragment and the acceptor vector pMXT32 (XOMA, Berkeley, Calif.; WO06/060769) were digested with BsmI and XhoI and ligated together resulting in the vector pTam29 (FIG. 15B).

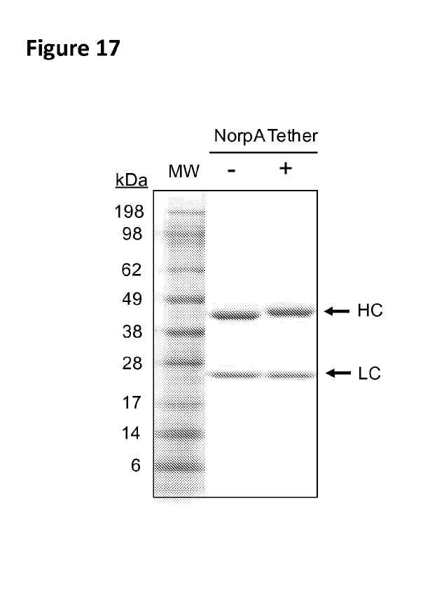

HEK 293E cells were transfected as described in Example 1. Ninety-six hours post transfection, cells were analyzed by flow cytometry for IL-1β binding and InaD PDZ1 expression (FIG. 16). As a control, cells transfected with pXIBM14 were negative for both IL-1β and c-Myc staining (A). As expected cells transfected with pTam29 were positive for c-Myc only (B). In contrast, cells co-transfected with: pXIBM32 and pTam29 (C); pXIBM34 and pTam29 (D); and pXIBM36 and pTam29 (E) were positive for both IL-1β binding and PDZ expression as measured by PE and Alexa647 fluorescence. In addition, the spacing between the C-terminus of XPA28IgG and NorpA tether did not seem to make a difference. These results successfully demonstrate that the NorpA tether display system is applicable to mammalian antibody display as well as yeast. In the absence of InaD PDZ1 expression, all three XPA28 IgG/NorpA tether proteins were secreted into the conditioned media and could be successfully purified using Protein A Sepharose indicating that the unpaired cysteine within the NorpA tether had no effect on the expression and folding of XPA28 IgG. Purification yields for the three fusion proteins were also comparable to XPA28 IgG without the NorpA tether. Reducing SDS-PAGE analysis of XPA28 IgG with and without the NorpA tether and GGGGS spacer confirmed of purity of the isolated proteins and presence of the NorpA tether fused to the heavy chain (FIG. 17).

### Example 6

### Testing the Importance of the InaD PDZ1/NorpA Disulfide Bond for Antibody Tethering

The crystal structure of the InaD PDZ1 domain (11-107) in complex with the C-terminal seven residues (1089-1095) of NorpA shows a disulfide bond between C31 and C1094 of InaD PDZ1 and NorpA, respectively (Kimple et al. 2001 EMBO J. 20:4414-4422). To investigate the importance of the disulfide bond in the tether display system, we constructed vectors pTam 49-52 by QuikChange™ mutagenesis using pTam37 as the template plasmid and the mutagenic primers outlined below:

pTam49 - Tam190
(SEQ ID NO: 81)
(GCCGGCAAGACCGAGTTCTCCGCCTGATGAGTTTAAACC)
and
Tam191
(SEQ ID NO: 82)
(GGTTTAAACTCATCAGGCGGAGAACTCGGTCTTGCCGGC).
pTam50 - Tam192
(SEQ ID NO: 83)
(CAAGAAGTCCTTCGGCATCTCCATAGTGCGCGGCGAGGTG)
and
Tam193
(SEQ ID NO: 84)
(CACCTCGCCGCGCACTATGGAGATGCCGAAGGACTTCTTG).
pTam51 - Tam220
(SEQ ID NO: 85)
(GTTATTGCCAGCGTTTTAGCATGATGAGCGGGTGAGCTCATTCAC)
and
Tam221
(SEQ ID NO: 86)
(GTGAATGAGCTCACCCGCTCATCATGCTAAAACGCTGGCAATAAC).
pTam52 - Tam222
(SEQ ID NO: 87)
(GTTATTGCCAGCGTTTTAGCATGATGACAGGTCCAGCTGGTGCAG)
and
Tam223
(SEQ ID NO: 88)
(CTGCACCAGCTGGACCTGTCATCATGCTAAAACGCTGGCAATAAC).

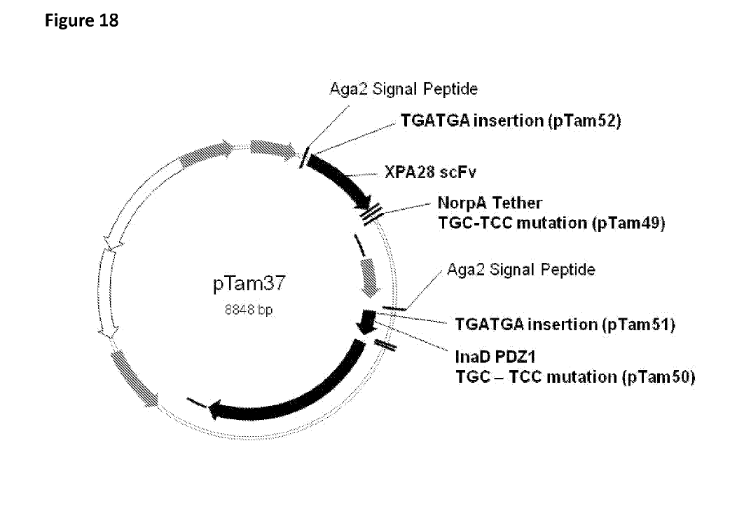

In pTam49, the penultimate cysteine residue (C1094) of the NorpA tether was mutated to a serine while the corresponding mutation (C31S) was made in the InaD PDZ1 domain resulting in pTam50 (FIG. 18). Both pTam51 and 52 contain two stop codons inserted immediately prior to the first codon of InaD PDZ1 and XPA28 scFv, respectively (FIG. 18).

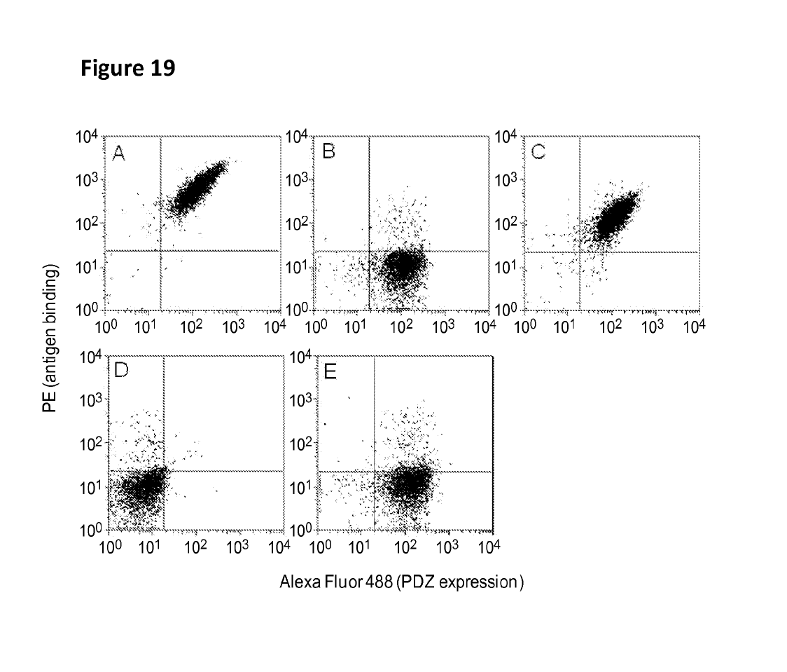

BJ5465 cells were transformed with pTam37, 49, 50, 51 and 52, and grown in SDCAA containing 40 μg/ml tryptophan prior to galactose induction. Cells were then analyzed for IL-1β binding as well as for the presence of c-Myc and HA epitope tags using flow cytometry. As shown in FIG. 19A, cells transformed with pTam37 bind IL-1β whereas cells transformed with the NorpA C1094S mutant (pTam49) do not (FIG. 19B). Interestingly, the InaD PDZ1 C31S mutant (pTam50) retains IL-1β binding, albeit at level lower than pTam37 (FIG. 19C). Thus, the data demonstrate the importance of the disulfide bond between C31 and C1094 of InaD PDZ1 and NorpA, respectively. In all three vectors, the presence of PDZ on the cell surface was observed by Alexa Flour® 488 fluorescence. Finally, IL-1β binding was absent in cells that lack InaD PDZ1 (pTam51) or XPA28 scFv (pTam52) (FIGS. 19D and E), indicating that there were no non-specific interactions between IL-10 and InaD PDZ1 or the cell surface.

### Example 7

### Construction of an Antibody Tether Display Library

The acceptor vector pTam48 was created by replacing XPA28 in pTam37 with a 1.5 kilobase stuffer DNA fragment. This vector was then digested with NheI and SfiI to remove the stuffer DNA and gel purified. Insert DNA corresponding to the scFv library was generated as follows: VH and Vλ regions were PCR amplified from cDNA isolated from bone marrow, PBMCs, or spleens of thirty healthy donors using primers designed from V-Base. Each family of VH (1-7) and VL (1-10) were individually amplified using forward primers that anneal to the V segment and reverse primers annealing in the VJ and CH1 region for VH and VL respectively. Secondary PCR reactions were performed in order to add a NheI restriction site to the 5′ end of VH and a SfiI restriction site to the 3′ end of the VL region. Additionally, the reverse secondary primer for VH and forward secondary primer for VL are complementary and encode for the glycine-serine linker. PCR products for VH were pooled based on subfamily (VH1-7) while a single pool was created for VL (VL total). DNA from VH1-7 and VL total were mixed at a 5:1 ratio and a single combined fragment corresponding to the scFv was generated by overlap extension PCR using the following assembly primers (yAFor: TAAGATGCAGTTACTTCGCTGTTTTTCAATATTTTCTGTTATTGCTAGCGTTTTAG C (SEQ ID NO: 89) and yARev: ATGATGTGCGGCCCCTCCCAGATCTTCTTCTGAGATGAGTTTTTGTTCCAGGCCCC CGAGGC (SEQ ID NO: 90)). Both assembly primers contain 48 complementary base pairs of homology to the linearized acceptor vector pTam48. The seven assembly PCR products (VH1-7/VL pool) were combined into a single pool according to the natural distribution as described in V-Base (MRC Centre for Protein Engineering, Cambridge, UK). Vector and insert DNA were combined by homologous recombination. Linearized vector (8 μg) and scFv DNA (24 μg) were electroporated into BJ5465 cells resulting in a library size of 1.74×10⁸ transformants.

### Example 8

### Isolation of Anti-Transferrin scFvs from an Antibody Tether Display Library

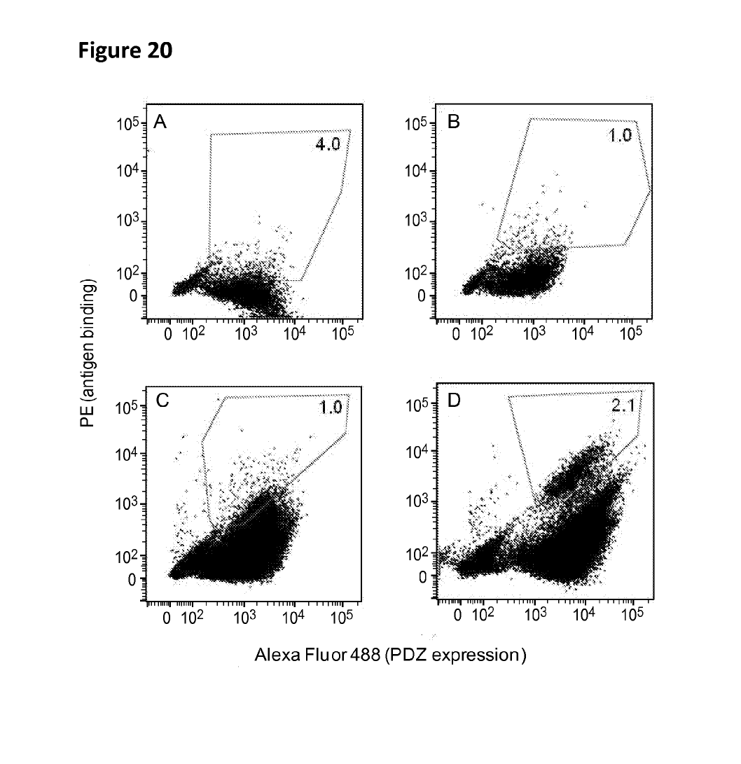

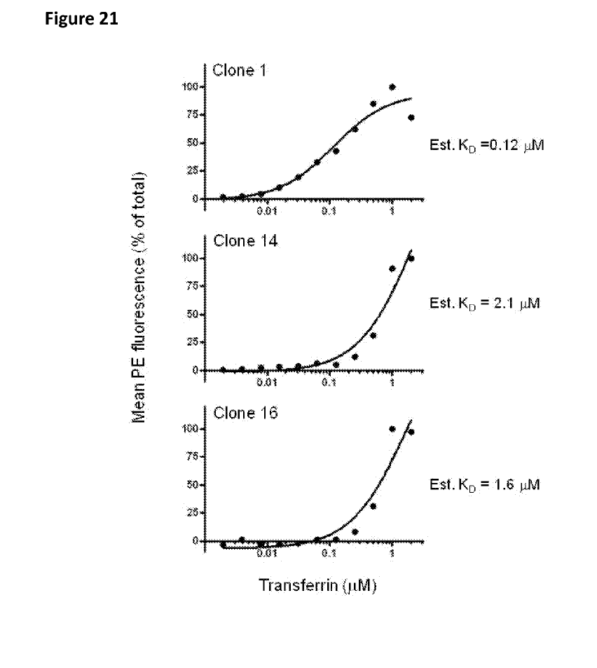

For the first round of panning, 1×10¹⁰ cells from the library described in example 7 were incubated with 1 μM of biotin-labeled transferrin in FACS buffer (PBS containing 0.1% BSA) for 1 hour at room temperature. The cells were washed and incubated with streptavidin magnetic micro beads (Miltenyi Biotec, Cologne, Germany) for 10 minutes on ice. The cell suspension was then added to a LS column (Miltenyi Biotec), washed with FACS buffer, and eluted into SDCAA media containing 40 μg/ml tryptophan, and grown for 16-24 hours. Subsequently, cells were then passaged and grown an additional 24 hours. In preparation for FACS, cells were then transferred into SGCAA media containing 40 μg/ml tryptophan and grown 20-24 hours. For the second round of panning, cells were incubated with anti-c-Myc (4 μg/ml) in addition to biotinylated transferrin for 1 hour at room temperature. Cells were then washed with cold FACS buffer and incubated with streptavidin-phycoerythrin (PE) (10 μg/ml), anti-chicken Alexa Fluor® 647 conjugate (20 μg/ml) and anti-hemagglutinin (HA) Alexa Fluor® 488 conjugate (10 μg/ml) for 30 minutes at 4° C. and protected from light. Labeled cells were sorted using a FACSAria™ instrument (BD, Franklin Lakes, N.J.). As shown in FIG. 20A, sort gates corresponding to 4.0% of the population were used isolate clones that were positive for both PE and Alexa Fluor® 488 fluorescence. Sorted cells were collected in SDCAA containing 40 μg/ml tryptophan and grown at 30° C. for the next round of panning. In subsequent panning rounds, the location of the sort gate was altered in order to isolate clones of greater fluorescence (FIG. 20 B-D). After four rounds of enrichment by FACS, single clones were isolated by plating and used to inoculate a single 96-well plate of media. Transferrin binding by the individual scFv clones was assessed by flow cytometry. Subsequently, 18 unique sequences were identified and further characterized using antigen titration curves. Cells were then incubated with increasing concentrations of biotin labeled transferrin (1 nM-2 μM) and analyzed by flow cytometry. The mean PE fluorescence (percentage of total) was plotted against antigen concentration and the dissociation constant (K_{D}) was determined as the concentration at the half maximum (EC₅₀). Estimated affinities for three exemplary clones are shown in FIG. 21.

### Example 9

### Display of IgG and Fab on Yeast Cell Surface

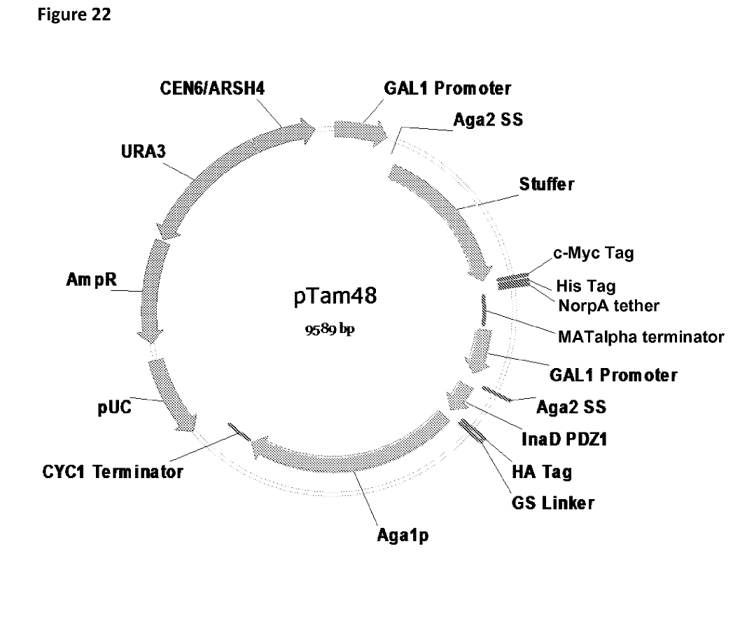

In order to display the antibody XPA28 IgG on the surface of yeast cells, the vector pTam48 (see Example 7) was first modified by QuikChange™ mutagenesis to silence the second PmeI site (FIG. 22), using primers Tam147 (CTAGGATCAGCGGGTTTAGACTTAACTGAAAATTACATTGC) (SEQ ID NO: 535 and Tam148 (GCCCTCTAGGATCAGCGGGAATTCTTAACTGAAAATTACATT) (SEQ ID NO: 536). The resulting vector was digested with NheI and PmeI to remove stuffer sequence, cMyc and His₆ epitope tags, and the NorpA tether. The resulting 8 kb fragment was then ligated to a 3 kb overlap extension PCR fragment that had been digested with NheI-HF and PmeI. The latter fragment was comprised of the following three PCR amplifications:
(1) the light chain from the vector pXIBM36 (see FIG. 15A), amplified with primers CAATATTTTCTGTTATTGCTAGCGTTTTAGCACAGGCTGTGCTGACTCAG C (SEQ ID NO: 537) and AGTCGATTTTGTTACATCCTATGAACATTCTGTAGGGGCCAC (SEQ ID NO: 538);
(2) the MATα terminator, Gal1 promoter, and Aga2 signal sequence from the vector pTam48, amplified with primers GATGTAACAAAATCGACTTTGTTCC (SEQ ID NO: 540) and GACTGCACCAGCTGGACCTGTGCTAAAACGCTGGCAATAAC (SEQ ID NO: 541);
(3) the V_{H} and C_{H1-3} region from the vector pXIBM36, amplified with primers CAGGTCCAGCTGGTGCAGTC (SEQ ID NO: 542) and TCAGATCAGCGGGTTTAAACTCAGGCGCAGAACTCGGTCTTG (SEQ ID NO: 543).

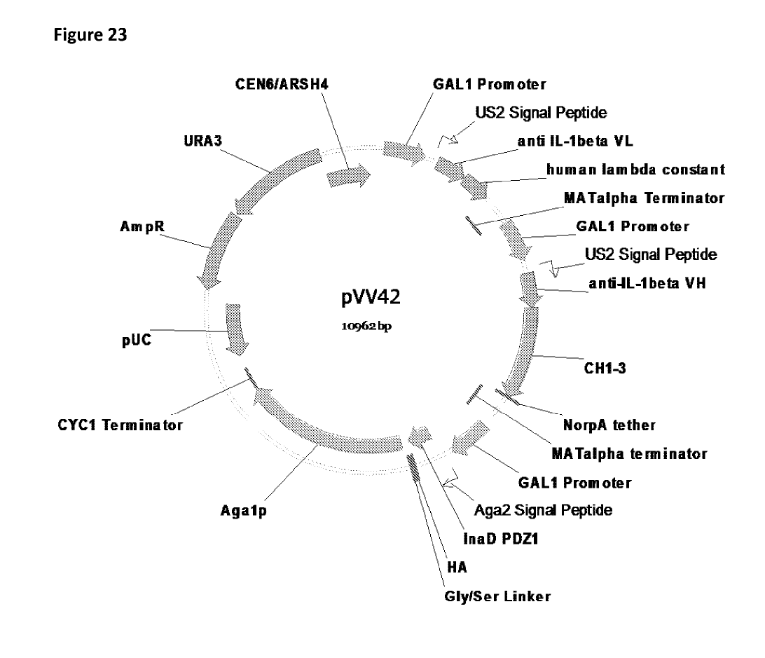

For cloning purposes, a NcoI site in the URA3 gene was silenced by QuikChange™ mutagenesis and primers CTTAACTGTGCCCTCCATCGAAAAATCAGTCAAGATATC (SEQ ID NO: 544) and GATATCTTGACTGATTTTTCGATGGAGGGCACAGTTAAG (SEQ ID NO: 545). The final vector, pVV42 is shown in FIG. 23.

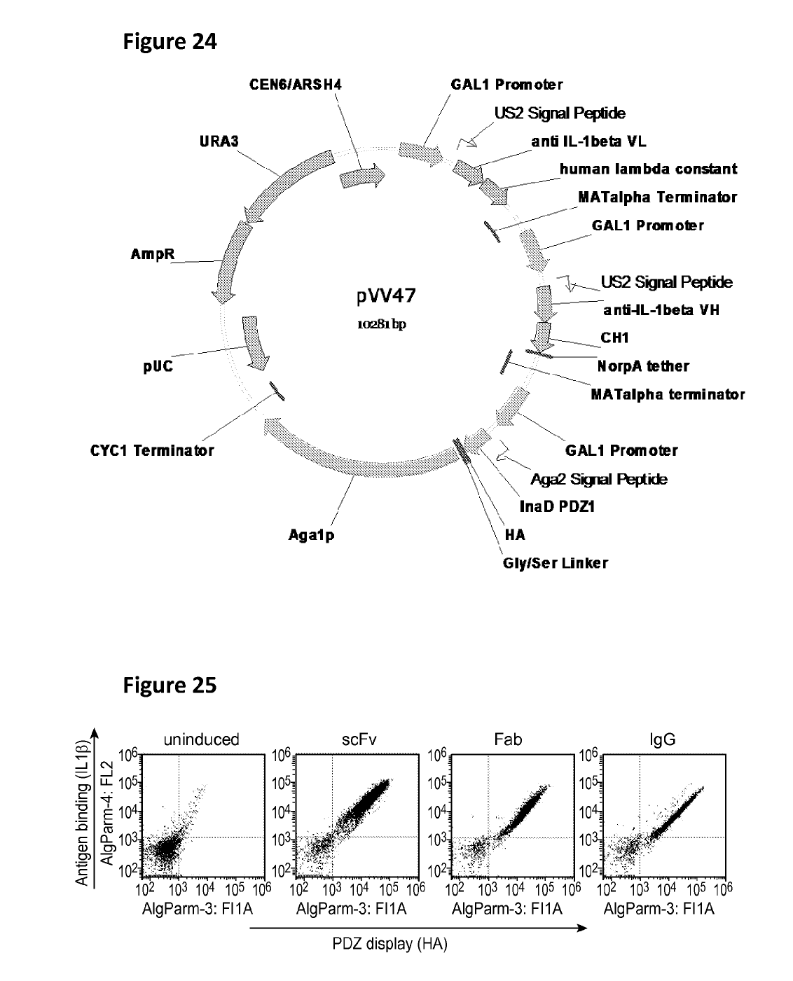

The sequence coding for the Fc region in the IgG vector pVV42 (FIG. 24) was removed by amplifying the V_{H} and C_{H}1 domains from vector pVV42 with primers oVV82PCR (CAAGCCATGGCTCAGGTCCA) (SEQ ID NO: 546) and oVV82PCRreverse (GGGTTTAAACTCAGGCGCAGAACTCGGTCTTGCCTGAACCGCCGCCTCCACAAG ATTTGGGCTCAACTCTCT (SEQ ID NO: 547), where the reverse complement of the underlined sequence codes for the NorpA tether amino acids GKTEFCA (SEQ ID NO: 16)). The PCR fragment was then digested with NcoI and PmeI and ligated into pVV42, which had been previously digested with NcoI and PmeI, resulting in a new vector, pVV47 (FIG. 24).

BJ5465 yeast cells were transformed with the following vectors: pTam37 (XPA28 scFv), pVV47 (XPA28 Fab), and pVV42 (XPA28 IgG1), grown on SDCAA containing 40 μg/ml tryptophan, and induced with galactose. For flow cytometry analysis, cells were washed with wash buffer, then incubated with biotin labeled IL-1β (100 nM) for 1 hour at room temperature. The cells were then washed and incubated with streptavidin-phycoerythrin (PE) (10 μg/ml) and anti-hemagglutinin (HA) Alexa Fluor® 488 conjugate (10 μg/ml) for 30 minutes on ice and protected from light. The cells were then washed one final time before being analyzed. Shown in FIG. 25 is the bivariate plot of PE and Alexa Fluor 488 fluorescence, indicating the presence of biotin-IL-1β and the InaD PDZ1/Aga1 fusion on the yeast cell surface, respectively. These results indicate that all three constructs produced displayed proteins that bound IL-1β. Cells displaying the scFv format exhibited the highest level of antigen binding among the three formats, followed by Fab, then IgG (FIG. 25). The level of PDZ/Aga1 display appears to be independent of antibody size and was similar for all three constructs. This experiment demonstrates that multiple antibody types can be displayed on the yeast cell surface using the disclosed PDZ tether display system.

### Example 10

### Identification and Characterization of Tie2 Binding Antibodies Using Integrated Phage, Yeast, and Mammalian Display

### Construction Of XFab 1 Library

The XFab1 phage library was prepared as follows: cDNA was prepared from 30 donors (AllCells, Emeryville, USA) by RT-PCR using standard methods (Sambrook et al., Molecular Cloning: A Laboratory Guide, Vols 1-3, Cold Spring Harbor Press (1989)). The VL and VH regions were PCR amplified using cDNA templates and primers based on the germ-line sequences from V BASE (MRC Centre for Protein Engineering, Cambridge, UK). Amplified VH and VL fragments were then ligated into pXHMV-US2-L-Fab or pXHMV-US2-K-Fab vector sequentially. Ligated DNA was electroporated into electrocompetent TG1 cells (Lucigen, Middleton, USA). The size of the XFab1 library obtained was 2.5×10¹¹ transformants.

### Phage Panning Against Tie2 Antigen

In preparation for phage panning, streptavidin-coupled Dynabeads® (Life Technologies, Carlsbad, Calif.) were washed 3× with PBS containing 5% milk. The beads and phage (100× library equivalent) were then blocked with PBS containing 5% milk for 1 hour at room temperature. A deselection step was performed by incubating the blocked phage with 100 μl of blocked beads for 30 minutes at room temperature. This step was repeated again. The antigen Tie2 (R&D System, Minneapolis, Minn.) was labeled with Sulfo-NHS-LC Biotin (Thermo Scientific, Rockford, Ill.). For the first round of panning, 100 pmoles of antigen was incubated with 100 μl of blocked beads for 30 minutes. Following washing, the Tie2 bound beads were incubated with the deselected library phage for 1 hour at room temperature. The beads were then washed 3× using PBS containing 0.05% Tween® 20 and 5% milk. Bound phage were eluted by suspending the washed beads in 500 μl of 100 mM TEA (EMD Chemicals, Gibbstown, N.J.) for 20 minutes and then neutralized with an equal volume of 1M Tris (pH 7.4) (Teknova, Hollister, Calif.). The phage/bead solution was then used to infect log phase TG1 E. coli (10 ml) cells for 1 hour at 37° C. with 100 rpm shaking. Infected cells were plated on 2YTCG media (2YT containing 100 g/ml carbenicillin and 2% glucose) and incubated at 30° C. overnight. The following day, the cells were collected by scraping and used to inoculate fresh liquid 2YTCG media.

Cells numbering 100× of the output phage from the previous round were used to inoculate a culture of 2YTCG cells for final optical density at 600 nm (OD₆₀₀) of 0.05. The culture was then grown at 37° C. with shaking (250 rpm) until an OD600 of 0.5 was reached. M13K07 helper phage (New England Biolabs, Ipswich, Mass.) was added to the culture at a multiplicity of infection (MOI) of 20. The culture was further incubated at 37° C. for 1 hour with shaking (100 rpm). Infected cells were then pelleted and resuspended in 2YT medium with 100 μg/ml carbenicillin and 50 μg/ml of kanamycin and grown overnight at 30° C. with shaking (250 rpm). The following day, the cells were pelleted and the phage supernatant was set aside. For the second round of panning, 1 ml of phage supernatant and 50 pmoles of biotin-labeled Tie2 were used. In the third round, 100 μl of phage supernatant was used while the amount of antigen remained unchanged (50 pmoles). All other panning conditions were similar to Round 1.

### Transfer of Enriched Phage Clones to Mammalian Display Vector

Infected cells from the third round of panning were grown on 2YTCG media overnight at 30° C. as before. The following day, cells were scraped and plasmid DNA was isolated using a Plasmid Mega kit (Qiagen, Valencia, Calif.). Plasmid DNA (30 μg) was digested with SfiI and NheI in order to excise a single insert containing the VL, CL and VH regions. The acceptor vector, pXIBM36 was also digested with the same restriction enzymes. Both insert and cut vector were gel purified using a QIAquick® gel extraction kit (Qiagen, Valencia, Calif.). One microgram of DNA total consisting of a vector-to-insert ratio of 2:1 was incubated overnight at 16° C. with T4 DNA ligase. One microliter of the ligation reaction was used to electroporate TOP10 E. coli (Life Technologies, Carlsbad, Calif.). After plating and overnight growth, colonies representing 10× of the third round phage output were scraped and plasmid DNA was isolated as before. DNA was then digested with AvrII and NcoI to remove a fragment containing an E. coli optimized CL and the pelB signal sequence preceding the VH region. This was replaced with a fragment containing a human CL, IRES2 and an IgG leader sequence by ligation and electroporating TOP10 cells as before. Again following plating and overnight growth, colonies representing 10× of the third round phage output were scraped and processed for DNA. The purified plasmid DNA now contained the phage-derived VL and VH regions transferred into an IgG1 backbone.

### Identification of Anti-Tie2 IgGs Using Mammalian Display

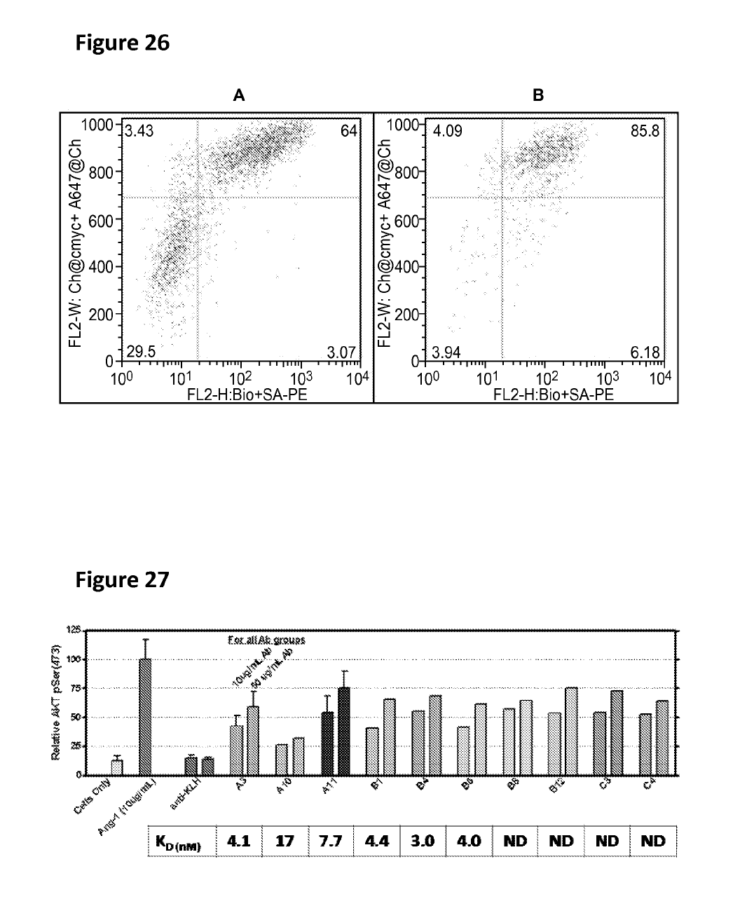

HEK293E cells were transfected as described in Example 1 using a 1:1 ratio of plasmid DNA encoding IgG and pTam29 for a total of 20 μg. Following a 24 hour transfection, cells were incubated with biotin-Tie2 at a final concentration of 5 μg/ml and chicken anti c-Myc (4 μg/ml) (Life Technologies, Carlsbad, Calif.) for 1 hour at 4° C. Subsequent labeling and flow cytometry analysis was performed as described in Example 1. As shown in FIG. 26A, the majority of the cells analyzed were positive for both Tie2 binding and PDZ display confirming that both the transfection and the 2-step cloning of VL and VH from phage vector into the mammalian display vector, were successful. Next, the transfected cells were subjected to another round of enrichment using magnetic cell separation (MACS). Briefly, cells (2×10⁷) were washed with PBS containing 0.5% FBS and 2 mM EDTA and incubated with 10 pmoles of biotin labeled Tie2 for 30 minutes at 4° C. The cells were washed again and incubated with 40 μl of anti-biotin microbeads (Miltenyi Biotec, Auburn, Calif.) for 15 minutes at 4° C. Cells were washed again and resuspended in PBS containing 0.5% FBS and 2 mM EDTA for a final volume of 500 μl. The cell suspension was then filtered using a 70 μm nylon mesh prior to loading onto a LS column (Miltenyi Biotech, Auburn, Calif.). Tie2-binding cells were isolated using a MidiMacs™ separator (Miltenyi Biotech, Auburn, Calif.) according to the manufacturer's instructions. Analysis of the eluted cells by flow cytometry revealed that the additional round of panning by MACS further enriched the percentage of Tie2-binding cells in the population by 20% (FIG. 26B) as compared to the previous panning round (FIG. 26A).

Plasmid DNA was isolated from the eluted cells using a Mini-prep kit (Qiagen, Valencia, Calif.), electroporated into TOP10 E. coli cells, and grown overnight at 37° C. on LB media containing 100 μg/ml carbenicillin. The following day, colonies were picked for sequence analysis, of which 34 were unique clones. In order to produce soluble IgG for further characterization, the following modifications were made to the transfection protocol described in Example 1.

Ten microliters of IgG miniprep DNA was incubated with 1 μg of Lipofectamine™ for 20 minutes at room temperature. The mixture was then added to 100 μl of HEK293E cells seeded in a 96-well culture plate. Following 48 hours of growth at 37° C. with 5% CO₂, the cell media was collected.

IgG clones were then screened for binding to Tie2 expressed on CHOK1 cells. To accomplish this, 25 μl of cell media containing secreted IgG supernatant was incubated with 25 μl of CHOK1-Tie2 cells (2×10⁶ cells/ml) for 30 minutes at 4° C. Prior to this, the CHOK1-Tie2 cells had been pre-labeled with CSFE dye (Life Technologies, Carlsbad, Calif.) to allow their discrimination by flow cytometry. The cells were then washed with FACS buffer (PBS containing 0.5% BSA and 0.01% sodium azide) and incubated with 25 μl of mouse anti c-Myc (400 ng/ml) (Roche Applied Science, Indianapolis, Ind.) in FACS buffer for minutes. The cell pellet was washed again and incubated with 25 μl of a 1:200 dilution of allophycocyanin (APC)-conjugated goat anti-mouse IgG (Jackson Immuno Labs, West Grove, Pa.) in FACS buffer for 15 minutes at 4° C. After labeling, the cells were resuspended in 50 μl of a 1:1 mixture of FACS buffer and 4% paraformaldehyde (Sigma Aldrich, St. Louis, Mo.) before flow cytometric analysis.

### Affinity Determination and Functional Characterization of Anti-Tie2 IgGs

Based on the CHO-Tie2 binding screen, the top ten IgG clones were propagated into 24-well culture plates and grown for an additional 3 days to produce IgG for affinity determination. Media containing secreted IgGs were first diluted to ˜2 μg/ml (total protein) with assay buffer (10 mM HEPES, 150 mM NaCl, 3 mM EDTA, 0.05% Surfactant P20, 1 mg/ml BSA, pH 7.4) and injected onto anti-human IgG (Jackson ImmunoResearch, West Grove, Pa.)-coupled biosensors using a Biacore A100 (GE Healthcare, Waukesha, Wis.). Six concentrations of Tie2 in serial three-fold dilutions (10 nM-0.04 nM) were then injected over the captured IgG in duplicate. Injections of Tie2 were 4 minutes at 30 μl/minute while the dissociation time was 10 minutes. Collected data was fit to a 1:1 Langmuir interaction model using Biacore A100 evaluation software (GE Healthcare, Waukesha, Wis.). Dissociation constants were calculated for six out of the ten IgG clones (FIG. 27) Impressively, all 6 IgGs displayed single to double digit nanomolar affinity for soluble Tie2.

In addition to Biacore, a functional assay was also performed on the top ten clones from the binding screen. Serum starved CHOK1-Tie2 cells were incubated with the Tie2 ligand, Ang1 (10 μg/ml), or anti-Tie2 IgGs (10 and 50 μg/ml) for 10 minutes at 37° C. Cellular Akt phosphorylation at serine 473 was then determined using Phospho-Akt kit (Meso Scale Discovery, Gaithersburg, Md.). As shown in FIG. 27, nine out of ten clones that bind Tie2 were found to activate Tie2 signaling. Phospho-Akt levels were observed to be 40-60% of those seen with Ang1. Negligible phosphorylation was observed for the negative controls: anti-KLH treated and untreated cells.

### Transfer of Enriched Phage Clones to Yeast Display Vector

Tie2 round 3 output from lambda Fab phage display library XFab1, consisting of 10⁵-10⁶ cfu, was cloned into the yeast display vector pVV42 (FIG. 23). In the first step, the SfiI/NheI fragment from the output was bulk-transferred by ligation (1:2 molar ratio of vector:insert) into similarly digested pVV42. Electroporation of 3 ul ligation (165 ng vector) into 40 ul XL1-Blue E. coli cells (Agilent, Santa Clara, Calif.) yielded ˜4×10⁹ transformants. In the second step of cloning, yeast sequences replaced the bacterial sequences between the variable domains; specifically, AvrII/NcoI fragment from pVV42 (consisting of CL-MATalpha terminator-Gal1 promoter-US2 signal sequence) was ligated 1:6 vector:insert into the similarly-digested library constructed in step one. Electroporation of 2.5 ul ligation (91 ng vector) into 50 ul TOP10 E. coli cells (Invitrogen, Carlsbad, Calif.) in each of 22 transformations yielded ˜4000 colonies.

Yeast were electroporated with the DNA isolated from these bacterial colonies. Yeast strain BJ5465 was made electrocompetent and transformed as described in Benatuil et al, Protein Engineering, Design & Selection 23, 155-9 (2010). DNA was concentrated with Pellet Paint®co-precipitant (EMD Chemicals, Darmstadt, Germany) and 2.5 μg electroporated in ˜3.2×10⁹ yeast cells, yielding ˜2×10⁵ yeast colony-forming units.

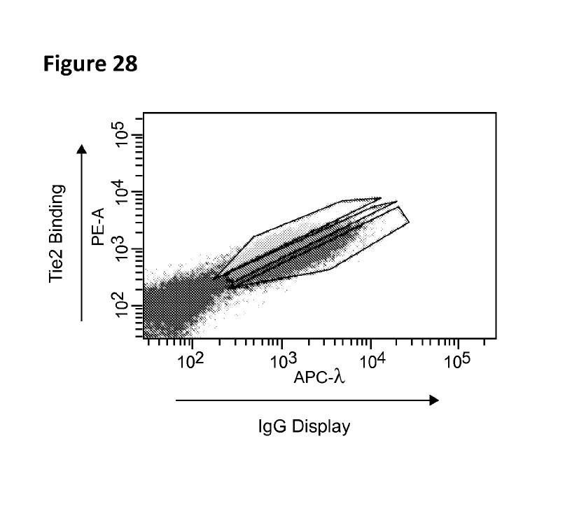

Yeast FACS, screening, and sequencing: Expression from library-transformed yeast was induced (after 48 hours in SDCAA+40 μg/ml tryptophan) by incubation for 26 hours in SGCAA+40 μg/ml tryptophan+galactose. Binding to antigen was detected by incubating yeast for one hour with ˜400 ng/ml biotinylated Tie2-Fc and then fluorescent PE-tagged streptavidin, as well as an antibody detector: 10 μg/mL fluorescent APC-tagged anti-lambda IgG (Brookwood Biomedical, Birmingham, Ala.). Labeled cells were sorted using a FACSAria™ instrument (BD, Franklin Lakes, N.J.). As shown in FIG. 28, three populations of Tie2-binding and antibody-displaying double positive cells were collected. Of 94 colonies then induced, all were verified as binding Tie2 by flow cytometry. Of 58 clones sequenced, 20 were unique, representing a diversity of families of heavy chain and light chain complementarity-determining regions.

### Analysis of Unique Clones Discovered by Using the Integrated Display Platforms

The sequences of the unique Fab clones that were discovered using only yeast display were compared with the unique Fab clones that were recovered using yeast or mammalian display. For each display platform, the majority of the clones were not recovered using the other display technologies (Table 3—the percentage of clones from each selection also seen using other selection methods is shown in unshaded boxes and the percentage of clones unique to one selection method is shown in the shaded boxes). Therefore, using an integrated display method allowed additional diverse clones to be discovered that were not accessible using a single platform.

TABLE 3

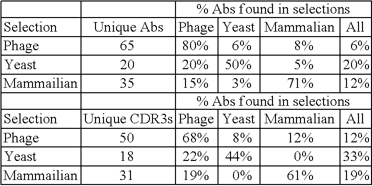

1. A host cell comprising:
(a) a polynucleotide encoding a cell surface protein fused to a PDZ Domain; and
(b) a polynucleotide encoding an antibody, or antigen-binding fragment thereof, fused to a PDZ-binding peptide.

2. The host cell of claim 1, where the PDZ-binding peptide is 5 to 20 amino acids in length.

3. The host cell of claim 1, wherein the cell is selected from the group consisting of a eukaryotic cell and a prokaryotic cell.

4. The host cell of claim 3 wherein the eukaryotic cell is a yeast cell or a mammalian cell.

5. The host cell of claim 4 wherein the yeast cell is selected from the group consisting of S. cerevisiae, P. pastoris, C. albicans, H. polymorpha, Y. lipolitica, and S. pombe.

6. The host cell of claim 3 wherein the prokaryotic cell is selected from the group consisting of E. coli, Salmonella typhimurium, Bacillus subtilis, Pseudomonas aeruginosa, and Serratia marcescans.

7. The host cell of claim 1, wherein the cell surface protein is a cell wall protein.

8. The host cell of claim 7, wherein the cell wall protein is selected from the group consisting of Aga1, Aga2, Agα1, Cwp1, Cwp2, Gas1p, Yap3p, Flo1p, Crh2p, Pir1, Pir2, Pir3, and Pir4.

9. The host cell of claim 1, wherein each of the PDZ Domain and the PDZ-binding peptide comprise a Cys residue.

10. The host cell of claim 1, wherein the polynucleotide of part (a) further encodes an enhancer domain.

11. The host cell of claim 10, wherein the enhancer domain is a variant of the 10^{th} fibronectin type III domain of human fibronectin (FN3).

12. The host cell of claim 1, wherein the PDZ Domain is selected from the group consisting of an InaD PDZ domain (SEQ ID NO: 2), a Dishevelled 1-like (DVL1L) PDZ (SEQ ID NO: 3), a proTGF-alpha cytoplasmic domain-interacting proteins 18 (TACIP18) PDZ1 (SEQ ID NO: 4), a similar to TACIP18 (SITAC) PDZ1 (SEQ ID NO: 5), a PSD-95/SAP90 PDZ3 domain (SEQ ID NO: 6), and an Erbin PDZ domain (SEQ ID NO: 7).

13. The host cell of claim 1, wherein the PDZ-binding peptide comprises a C-terminal sequence of NorpA (SEQ ID NO: 1) or a fragment thereof at least 90% identical.

14. The host cell of claim 13, wherein a Cys residue is located at the −1 position.

15. The host cell of claim 1, wherein the PDZ-binding peptide sequence is GKTEFCA (SEQ ID NO: 16).

16. The host cell of claim 1, wherein the PDZ-binding peptide is fused to the C-terminus of the antibody or antigen-binding fragment thereof.

17. The host cell of claim 1, wherein the polynucleotide of part (a) and/or the polynucleotide of part (b) further encodes a fluorescent marker protein.

18. The host cell of claim 1, wherein the polynucleotide of part (a) is integrated into the host cell genome.

19. The host cell of claim 1, wherein the polynucleotides of part (a) and part (b) are in separate vectors, or optionally in the same vector.

20. The host cell of claim 1, wherein the antibody is a tetrameric IgG immunoglobulin comprising two heavy chains and two light chains.

21. The host cell of claim 1, wherein the antigen-binding fragment of the antibody comprises at least the heavy chain variable region and/or the light chain variable region.

22. The host cell of claim 21, wherein the antigen-binding fragment of the antibody comprises a Fab or a scFv.

23. (canceled)

24. The host cell of claim 1, wherein the polynucleotide of part (a) further comprises a signal sequence directing the cell surface protein to the cell surface.

25. The host cell of claim 24, wherein the signal sequence is an Aga2 signal sequence when the host cell is a yeast cell.

26. The host cell of claim 1, wherein the PDZ-binding peptide is less than 15 amino acids in length.

27. The host cell of claim 1, wherein the PDZ Domain is about 80 to 100 amino acids in length.

28. The host cell of claim 1, wherein the PDZ Domain-PDZ binding peptide interaction has a K_{d} of about 100 nM or less.

29. A plurality of cells comprising at least 10̂3 different eukaryotic host cells according to any of the above claims, each such eukaryotic host cell expressing on its surface a different antibody, or antigen-binding fragment thereof.

30. The plurality of cells of claim 29 wherein the eukaryotic host cells are yeast cells or mammalian cells.

31. (canceled)

32. A method of displaying at least 10̂3 different antibodies, or antigen-binding fragments thereof, on cell surfaces, comprising culturing the plurality of cells of claim 29.

33. The method of claim 32, wherein the PDZ Domain and the PDZ-binding peptide are connected by at least one disulfide bond.

34. The method of claim 32, further comprising contacting the plurality of cells with an antigen, and optionally selecting cells which bind to the antigen.

35-37. (canceled)

38. A method of selecting an antibody, or antigen-binding fragment thereof, comprising:
(a) contacting a plurality of phage displaying antibody or antigen-binding fragments with an antigen, and selecting phage which bind to the antigen, and
(b) contacting the plurality of yeast cells of claim 30 with said antigen, and selecting cells which bind to the antigen.

39. A method of selecting an antibody, or antigen-binding fragment thereof, comprising:
(a) contacting a plurality of phage displaying antibody or antigen-binding fragments with an antigen, and selecting phage which bind to the antigen, and
(b) contacting the plurality of mammalian cells of claim 30 with said antigen, and selecting cells which bind to the antigen.

40. A method of selecting an antibody, or antigen-binding fragment thereof, comprising:
(a) contacting the plurality of yeast cells of claim 30 with an antigen, and selecting cells which bind to the antigen, and
(b) contacting the plurality of mammalian cells of claim 30 with said antigen, and selecting cells which bind to the antigen.

41. The method of claim 40 further comprising the step of contacting a plurality of phage displaying antibody or antigen-binding fragments with an antigen, and selecting phage which bind to the antigen.
